(19) United States
(12) Patent Application Publication
(10) Pub. No.: US 2022/0333147 A1
(43) Pub. Date: Oct. 20, 2022

(54) METHODS AND COMPOSITIONS RELATED TO PRINTED CELLULAR NICHES FOR PRESERVING AND CONTROLLING SYNTHETIC MICROBIAL CONSORTIA

(71) Applicants: BOARD OF REGENTS, THE UNIVERSITY OF TEXAS SYSTEM, Austin, TX (US); UNIVERSITY OF WASHINGTON, Seattle, WA (US)

(72) Inventors: Hal ALPER, Austin, TX (US); Shuo-Fu YUAN, Austin, TX (US); James WAGNER, Lawrence Township, NJ (US); Trevor JOHNSTON, Seattle, WA (US); Alshakim NELSON, Seattle, WA (US)

(21) Appl. No.: 17/620,927

(22) PCT Filed: Jun. 25, 2020
(86) PCT No.: PCT/US2020/039572

§ 371 Date: Dec. 20, 2021

Related U.S. Application Data
(60) Provisional application No. 62/867,475, filed on Jun. 27, 2019.

(51) Int. Cl. C12P 21/02; C12N 1/16; C12N 1/04; C12N 11/10; C12N 15/81

(52) U.S. Cl. CPC C12P 21/02; C12N 1/16; C12N 1/04; C12N 11/10; C12N 15/815; C12N 2513/00; C12N 2800/102; C12N 2533/74

(57) ABSTRACT

The present invention relates to a composition comprising a 3D printed hydrogel, wherein at least two different populations of cells are embedded in the 3D printed hydrogel. The different populations of cells can produce one or more products. The 3D printed hydrogel can be lyophilized and rehydrated, and the cells can continue to produce the product. Also disclosed are methods of producing a product, and methods of producing a 3D printed hydrogel comprising different populations of cells.

### CROSS-REFERENCE TO RELATED APPLICATIONS

This application claims benefit of U.S. Provisional Application No. 62/867,475, filed Jun. 27, 2019, incorporated herein by reference in its entirety.

### BACKGROUND OF THE INVENTION

Microbial production of value-added products ranging from small molecules to complex proteins is becoming increasingly attractive and effective across industry and academia (Lee 2017; Cordova 2019). Recent advances in synthetic biology have further enabled this bioconversion to be modular and distributed across multiple organisms, thus creating synthetic consortia that can reduce metabolic loads and afford more robust cell populations (McCarty 2019; Zhang 2019). However, most mono- and co-culture bioprocess applications rely on large-scale suspension fermentation technologies that are not easily portable, reusable, or suitable for on-demand production. These limitations are especially poignant when attempting to control the dynamics of a multi-organism consortium. Specifically, liquid co-cultures typically fail over time without sophisticated genetic control systems or particular nutrient conditions that seek to minimize the competitive growth bias that often occurs when utilizing disparate microorganisms (Bittihn 2018); Zhou 2015). Immobilized cell technologies, wherein microbes are encapsulated within a polymeric matrix, have been developed as an alternative to suspension cell culture (Niwas 2014; Kumaravel 2013; Schaffner 2017; Saha 2018). These microbe-laden matrices have been used to investigate quorum sensing between microbial species (Connell 2013), and as 3D architected ‘living materials’ (Schaffner 2017; Saha 2018). Calcium alginate and other polysaccharides are the most common matrices used for immobilizing cells, despite the sensitivity of the ionic cross-links to the presence of charge-bearing molecules and the pH of the medium (Cheetham 1979).

What is needed in the art are methods and platforms that can spatially organize individual microbes and consortium members via direct-write 3D printing of microbe-laden hydrogel inks. Also needed are methods and systems for controlling cellular dynamics and consortia of cellular organisms, and for preserving those cellular organisms.

### SUMMARY OF THE INVENTION

The present invention relates to compositions and methods for 3D printed hydrogels comprising cells. Disclosed herein is a composition comprising a 3D printed hydrogel, wherein at least two different populations of cells are embedded in the 3D printed hydrogel. The cells can comprise eukaryotes, prokaryotes, or a combination of both. The cells can comprise any type of cell, such as eukaryotic cells such as mammalian cells, yeast, or fungi, as well as prokaryotic cells such as bacteria or archaea, or a combination of two or more of these. At least one of the populations of cells can produce at least one product. The product can be anything capable of being produced by a cell, such as a protein, peptide, or small molecule. In one embodiment, at least both a first and second population of cells produces a product. In another embodiment, two or more different products are produced by the same cell within the hydrogel.

Also disclosed herein is a composition comprising a lyophilized and rehydrated 3D printed hydrogel, wherein at least one population of cells are embedded in the 3D printed hydrogel, and wherein the at least one population of cells is capable of producing at least one product; and further wherein the at least one product is capable of being produced at a rate of 50% or higher compared to production of the same product produced from an identical population of cells, present in substantially similar numbers, in an identical 3D printed hydrogel, wherein the cells in the identical 3D printed hydrogel are not lyophilized or rehydrated.

After lyophilization or preservation and subsequent rehydration, at least one product can continue to be produced at a rate of 50% or higher as compared to production of at least one product prior to lyophilization and rehydration of the composition. This can occur for at least a year or more after lyophilization and rehydration. At least one product can continue to be produced at a rate of 50% or higher after more than one lyophilization and rehydration cycle.

At least the first and second population of cells can produce different products. In one embodiment, the first population of cells produces a product which is consumed by the second population of cells. In a further embodiment, at least one population of cells embedded in the hydrogel can produce a product for at least 30 consecutive days.

The cells can be spatially organized in the 3D printed hydrogel. For example, at least two different populations can occupy at least two different spatial areas in the hydrogel.

The 3D hydrogel can comprise a polymer of Formula (I):

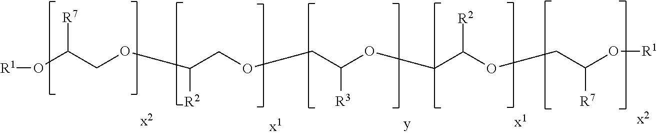

wherein
R¹ is hydrogen or a group having the formula:

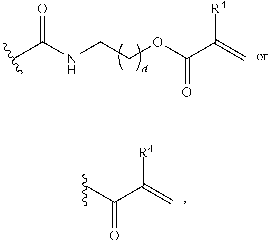

wherein d is 1, 2, 3, 4, 5, 6, 7, 8, 9, 10, 11, or 12; and R⁴ is hydrogen or methyl;
R² is hydrogen or —CH₂OR⁵; wherein R⁵ is C₁₋₁₂alkyl, C₁₋₁₂alkyl-OR⁶ or C₁₋₁₂alkyl-NR⁶₂, wherein each R⁶ is independently hydrogen or C₁₋₁₂alkyl;
R³ is hydrogen or methyl; and
R⁴ is hydrogen or methyl;
wherein R⁷ is —CH₂—O—C₁₋₁₂alkenyl;
y is selected to provide a block polymer with an Mₙ of about 500 to about 50,000;
x¹ and x² are selected to provide a block polymer with an Mₙ of about 100 to about 30,000;
x¹ is from 10-100; and y is from 25-250, provided that y is greater than x¹.

The 3D hydrogel can also comprise a polymer having the structure of Formula (II):

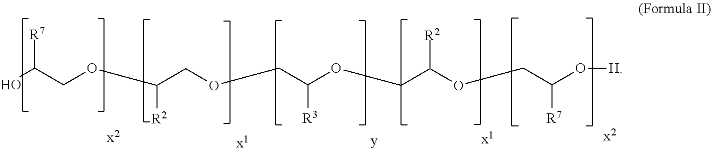

R² can be —CH₂—O—C₁₋₆alkyl; and R⁷ can be —CH₂—O—C₂₋₆alkenyl.

The polymer can have the structure of Formula (IIa):

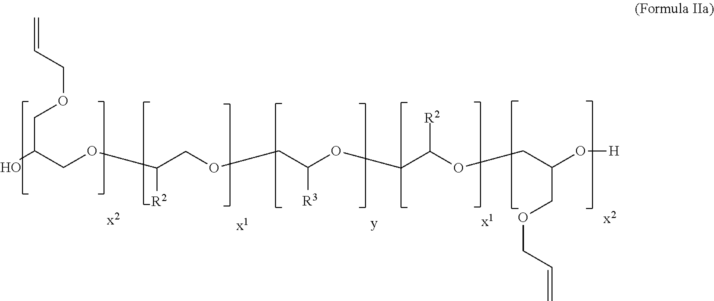

R² can be —CH₂—O—C₁₋₆alkyl.

The polymer can have the structure of Formula (IIb):

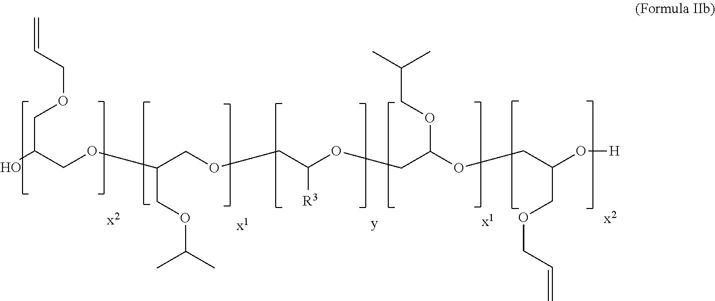

The polymer can have the structure of Formula (IIc):

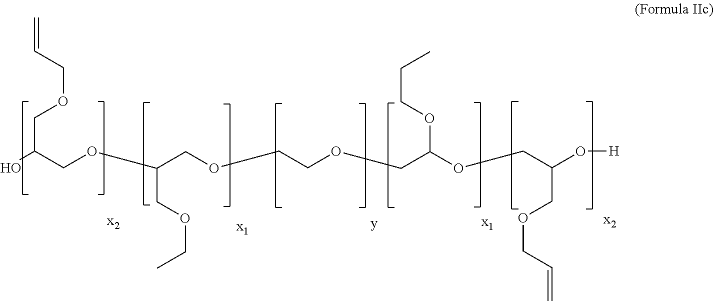

The polymer can have the structure of Formula (III):

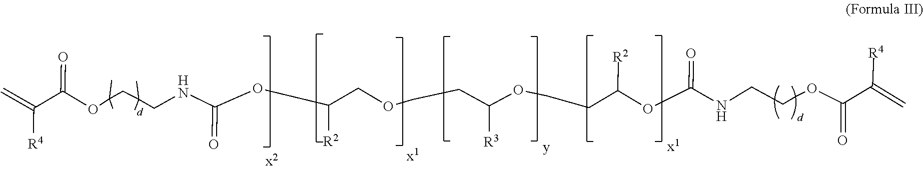

R² can be —CH₂—O—C₁₋₆alkyl.
The composition can have the structure of Formula (IIIa):

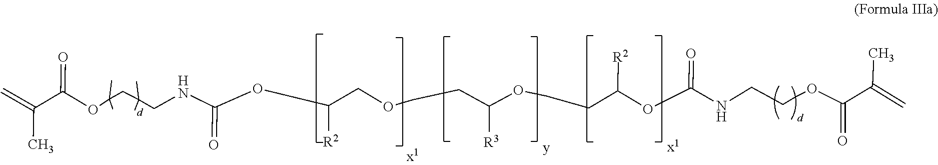

The polymer can have the structure of Formula (IIIb):

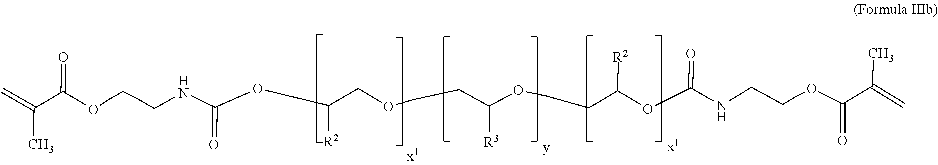

The polymer can have the structure of Formula (IIIc):

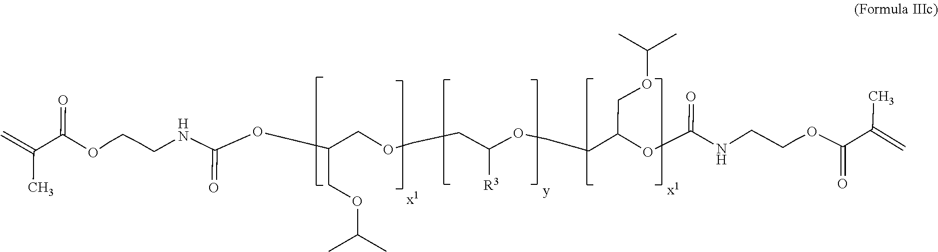

The 3D printed hydrogel disclosed herein can be in an aqueous media. The 3D printed hydrogel disclosed herein can further comprise a photoinitiator. The 3D printed hydrogel can be crosslinked.

Also disclosed is a bioreactor comprising any of the compositions disclosed herein.

Further disclosed is a method of producing a product from a cell, the method comprising: providing a composition comprising a 3D printed hydrogel wherein at least two different populations of cells are embedded in the 3D printed hydrogel, and further wherein at least one of the populations of cells produces a product; and exposing the composition to conditions favorable to produce the product.

At least two different populations of cells can be embedded in the hydrogel to produce a product. The products of these different organisms can be the same or different. In one embodiment, a first population of cells produces a product relied upon by the second population of cells.

Conditions of the composition disclosed herein can be adjusted to maximize production of at least one product produced by at least one population of cells in the composition. At least one of the population of cells can produce a product for at least 7 consecutive days. The composition can be preserved, such as by dehydration or lyophilization and reconstitution prior to producing a product. At least one of these products can continue to be produced at a rate of 50% or higher after lyophilization and rehydration of the composition as compared to production of at least one product prior to lyophilization and rehydration of the composition. In another embodiment, at least one product can continue to be produced at a rate of 50% or higher after lyophilization and rehydration of the composition at least a year or more after lyophilization and rehydration. For example, at least one product can continue to be produced at a rate of 50% or higher after more than one lyophilization and rehydration cycle.

Further disclosed is a method of producing a composition comprising a 3D printed hydrogel, wherein the 3D printed hydrogel comprises at least two different populations of cells; the method comprising: providing a shear-thinning hydrogel for each population; embedding in said hydrogel at least two different populations of cells; 3D printing said hydrogel comprising at least two different populations of cells onto a suitable substrate, thereby providing a composition comprising a 3D printed hydrogel. In one embodiment, the cells can be expanded by growing them. In another embodiment, the composition can preserved, such as by lyophilization, and reused.

In the methods disclosed herein, the 3D printed hydrogel can be cross-linked. At least two different populations of cells can form a consortia. At least two different populations of cells can be embedded in at least two separate shear-thinning hydrogels. Interactive dynamics between the at least two different populations of cells embedded in the at least two separate shear-thinning hydrogels can be controlled. For example, interactive dynamics can be controlled by printing the two separate hydrogels in different amounts. Interactive dynamics can be controlled by controlling amounts of individual cells embedded in each shear-thinning hydrogel. Interactive dynamics can be controlled by controlling conditions under which the composition is maintained. The gel composition and amount can be varied to control the population dynamics. For example, the total gel mass can be held constant and the ratio of a first population can be adjusted to alter end dynamics of a consortia. Conditions such as temperature and cell media can also be altered.

The composition comprising the 3D printed hydrogel can be preserved. In one embodiment, the composition is preserved by lyophilization and subsequently reconstituted. After lyophilization and rehydration, at least one product can continue to be produced at a rate of 50% or higher as compared to production of at least one product prior to lyophilization and reconstitution of the composition. At least one product can continue to be produced at a rate of 50% or higher after lyophilization and reconstitution of the composition at least a year or more after lyophilization and reconstitution. At least one product can continue to be produced at a rate of 50% or higher after more than one lyophilization and reconstitution cycle.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### DESCRIPTION OF THE FIGURES

FIG. 1 shows an overview of microbe-laden, 3D-printed hydrogels for on-demand production. The hydrogel encapsulation and on-demand production process is divided into three parts. In the Gel Preparation stage, the 3D printed and UV-cured microbial hydrogels are transferred to culture medium for cell outgrowth. While this initial outgrowth can also be used for production, the resulting cell-laden living materials can proceed to either the Gel Storage or On-Demand Production phase depending on user needs. In the Gel Storage stage, the microbial gels are treated with different types of preservation methods (such as lyophilization) for storage and future use. The preserved gels are subsequently rehydrated and incubated in fresh medium to perform on-demand production, with iterative re-uses as desired.

FIG. 2A-D shows re-use and preservation of mono-culture microbe-laden hydrogels. The fermentation performance of the printed hydrogel inks was tested for both bacterial (E. coli) and yeast (S. cerevisiae)-laden gels testing the production of 2,3-butanediol (A), L-DOPA (B), ethanol (C) and a peptide antibiotic (D). The production (pre- and post-preservation) is demonstrated in (A), (B), and (D). The production of ethanol from a year-long fermentation re-use process was evaluated using cell-laden hydrogel lattices (pictured top left in C).

FIG. 3A-E shows spatially organized consortia in hydrogels outperforms tradition liquid co-culture systems. (A) Images of cell-culture segregation in 3D printed samples of F127-BUM hydrogel. Left: a camera image of alternating stripes of RFP yeast and GFP bacteria, under irradiation with UV light. Right: confocal microscopy showing a z-stack of 100 microns of depth at the interface between gel samples printed with RFP yeast and GFP bacteria. Little to no movement of cells out of their designed boundaries is observed at the interface, indicating successful segregation of the microbes to their respective hydrogels. (B) Schematic metabolic pathway of a cross-feeding consortium E. coli-yeast for betaxanthins production. (C) Heat maps of the consortia performance of betaxanthins production in hydrogel system and liquid-based culturing. The productions are evaluated through altering cell number/gel ratios and fermentation temperatures. (D) The reusability of 30° C. hydrogels with 6:1 (yeast:E. coli) gel ratio for betaxanthins production between pre- and post-lyophilization processes are compared. (E) Glucose/xylose utilization via a parallel consortium with repeated uses. The consortia activity for glucose/xylose utilization between hydrogel system and liquid cultures are compared.

FIG. 4A-B shows gel-re-run for 2,3-butanediol (BDO) production. (A) The comparison of 2,3-BDO production between S. cerevisiae BY4741 and CEN.PK2-a strains before and after lyophilization. (B) The comparison of by-product acetoin formation between BY4741 and CEN.PK2-a strains before and after lyophilization. Each data point and error bar represent means and standard deviations from biological triplicates, respectively.

FIG. 5 shows L-DOPA production in E. coli. The comparison of L-DOPA production between engineered E. coli strains and control strain eBL0400DT. The highest L-DOPA producer eBL0430D strain was used for DOPA and betaxanthins experiments. Each data point and error bar represent means and standard deviations from biological triplicates, respectively.

FIG. 6A-B shows the reusability of yeast-laden hydrogels for a yearlong ethanol fermentation. Relative ethanol production (%; A) and titer (g/L; B) in yeast-embedded polymers are measured for each round of reuse. Data points (n=4 biological replicates) collected from each round of reuse are shown.

FIG. 7A-C shows gel-re-run for colicin V (ColV) peptide antibiotic production. (A), The bactericidal mechanism of ColV. Zone of inhibition test (B) and broth test (C) are used for evaluating antimicrobial activity of ColV in gel system and liquid culture before and after lyophilization. The inconsistency in toxin production by liquid culture suggested that liquid culture might be unstable and subject to random dynamics. Each data point and error bar represent means and standard deviations from biological triplicates, respectively.

FIG. 8A-C shows co-culture flow cytometry. (A) Visual confirmation of fluorescence and mixed culture under fluorescence microscope. (B) SSC versus FSC plot for distinguishing and counting E. coli and yeast using BD Fortessa flow cytometry. (C) Impact on consortia dynamics for bulk culture with different temperatures. Each data point and error bar represent means and standard deviations from biological triplicates, respectively.

FIG. 9A-D shows cell segregation and confluence in 3D-printed co-culture gels. (A) Confluence analysis results for the optimal E. coli growth condition (37° C.) in consortia hydrogels (left) and optimal yeast growth condition (25° C.) in consortia hydrogels (right). The E. coli and yeast samples achieved a confluence of 88.6% and 93.5% on day 7, respectively, in the hydrogel-based consortia relative to the optimal mono-culture gels. (B, panels i-vi) Standard images used in the confluence analysis of cell growth in consortia hydrogels, representing the ideal growth conditions for yeast and E. coli monocultures (30° C. in SC media, and 37° C. in LB media, respectively). (C), panels i-ii show images used for calculation of percent confluence of the microbial consortia hydrogels, at listed temperatures and days of incubation. (D), panels i-iii shows images depicting the cell segregation of bacteria and yeast in their respective hydrogel samples, with little to no movement or mixing of colonies between the hydrogels. The images were captured using confocal microscopy, showing a z-stack of 100 microns of depth at the interface between gel samples printed with RFP yeast and GFP bacteria. All images have 200-micron scale bars.

FIG. 10A-V shows betaxanthins production. (A) Medium optimization for betaxanthins production with different temperatures. Yeast sBY08 and E. coli eBL0430D strains were used herein. Production was evaluated using LBYSD (left, i) or M9YSD medium (right, ii) supplemented with 20 g/L glucose and appropriate amount of antibiotics. (B) The comparison of betaxanthins production for bulk culture between tube and flask scales. Betaxanthins fluorescence was measured at 24 and 72-hour time points. While production at flask scale works better than that at tube scale, probably because flask provides more oxygen transfer (DOD reaction requires oxygen), tube scale was chosen for the following experiments for operational convenience. (C) Betaxanthins production via DOD yeast-laden hydrogel. The comparison of betaxanthins production for yeast-laden gel between 3 mL and 5 mL gel culture volume. 3 mL culture volume was selected for the following experiments due to higher betaxanthins production possibly resulted from better oxygenation compared to that using 5 mL culture condition. (D-G) Betaxanthins production via E. coli-yeast consortia gels in round 0. The productions are evaluated through altering gel ratios and fermentation temperatures. Time course of betaxanthins production for hydrogel system at 25 (D), 30 (E), 33.5 (F) and 37 (G) ° C. (H-K), Betaxanthins production via E. coli-yeast bulk culture in round 0. The productions are evaluated through altering cell number and fermentation temperatures. Time course of betaxanthins production for bulk culture at 25 (H), 30 (I), 33.5 (J) and 37 (K) ° C. (L), The comparison of maximum production of betaxanthins in the round 0 between hydrogel system (top) and liquid culture (bottom). m-q, Gel-re-run for betaxanthins production via E. coli-yeast consortia. Cell-laden gels were removed from round 0. The performances between each sample were compared at 21 (m) and 96 hr (n) fermentation for the 1st round of gel-re-use. (O) The consortia activity between the 1st and 2nd round of gel-re-use were compared. Liquid culture in the 2nd round incubated at 30° C. for 21 hr (bottom) produced more betaxanthins than that in the 1st round (top). These results show that liquid culture can't easily control the consortia dynamics for the cell recycle batch fermentation purpose. The comparison of maximum betaxanthins production at 30 (P) and 33.5° C. (Q) between gel and liquid culture for 5 consecutive cell-reuse cycles (data points collected from (P and Q). (R-S)— shows the stability of multiple rounds of gel re-use compared to liquid culture. (T-U)— Investigation of consortia activity for betaxanthins production after preservation process. Lyophilization is one example (T). Three different preservation methods including lyophilization, refrigerated storage and liquid nitrogen freezing were applied to preservation of consortia-laden polymers. Additional five consecutive uses after preservation process were performed (data points collected from (U). 30° C. gels after round 5 were split (n=1) for examining the impact of lyophilization and refrigerated storage on betaxanthins production (s). (V) The comparison of maximum betaxanthins production between F127-BUM and calcium alginate hydrogels. Results from the comparison between our F127-BUM hydrogels, and commonly employed calcium alginate hydrogels show that the F127-BUM system is much more effective in the production and release of betaxanthins (2.53-fold increase in efficiency) over a period of 72 hours. Each data point and error bar represent means and standard deviations from biological triplicates, respectively, unless stated otherwise.

FIG. 11A-C shows gel-re-run for xylose/glucose utilization via a parallel yeast-yeast consortium. (A) Investigation of consortia activity on glucose/xylose utilization in hydrogel system and liquid culture with re-use. Round 0 was grown in YPD containing 20 g/L glucose to outgrow consortia population. Then all samples were cultivated in YPDX media for three consecutive uses. Xylose consumption rate for each round of re-use was compared. (B) Time profile of residual xylose concentration for each round of re-use (round 0 was grown in YPD (left) or YPDX (right)). (C) Time profile of xylose consumption for each round of re-use (round 0 was grown in YPD (left) or YPDX (right)). Each data point and error bar represent means and standard deviations from biological triplicates, respectively.

FIG. 12 shows SEAP production in hydrogels. Day 1 samples were analyzed. 2×10⁶ viable cells of wild-type CHO-DG44 were suspended in 100 μL of growth medium, embedded in 0.3 gram of polymer and served as a control. 2×10⁶ viable cells of SEAP-producing CHO-DG44 were individually suspended in 50 or 100 μL of growth medium and encapsulated in 0.3 gram of polymer. Maximal luminescence readout was obtained with reading gain 200 and emission at 540 nm.

FIG. 13 shows an overview of Bio-POD bioprocessing for on-demand protein production. Strain development of secreted proteins in engineered P. pastoris is achieved through auxotrophic or antibiotic selection coupled with appropriate biocatalytic screening assays. The engineered yeast cells are then encapsulated within the hydrogel and extrusion printed using syringes. The printed and UV-cured yeast-laden hydrogels are subsequently transferred to culture medium for cell expansion/enzyme production. The hydrogels can optionally proceed to lyophilization for storage after repetitive uses of the living microbial materials. Next, the preserved gels can be rehydrated in fresh medium for on-demand protein production, with iterative re-uses depending on user needs.

FIG. 14 A-D shows generation of SEAP-producing P. pastoris and SEAP production in hydrogel system. (A) Comparison of SEAP production between 48 hours and 72 hours fermentation at 30° C. P. pastoris Pp01 and Pp02 were used as the control and SEAP-producing strain, respectively. 0.5% methanol was added every 24 hours to maintain induction. (B) SDS-PAGE analysis of recombinant SEAP production. The protein marker was loaded into M lane of SDS-PAGE and supernatant of pelleted yeast containing SEAP was loaded into S lane. The arrow on the right indicates the size of SEAP (around 60 kDa) secreted to the media. (C) SEAP production assessed both pre- and post-lyophilization (data from round 2 and round 4 of reuse respectively) from hydrogels. (D) SEAP production in hydrogels (bars on left of each round) with repeated use compared to liquid culture (bars on right of each round) performance. All the samples were treated with lyophilization after round 3 of reuse. Data are mean±s.d.; n=6 biological replicates for hydrogels n=3 biological replicates for liquid culture.

FIG. 15 A-C shows generation of α-amylase-producing P. pastoris and α-amylase production in hydrogels system. (A) 10 out of 48 zeocin-resistant transformants were cultured in a 96-deep-well microplate and selected based on the cell growth. Secreted α-amylase capacities were evaluated via starch agar plate (measuring the size of the halos) and plate-based starch-iodine assay, where dark wells contain no amylase and lighter colored wells ranging from light blue to yellow contain increasing amounts of active amylase. Finally, the highest amylase producer was selected. (B) SDS-PAGE analysis of recombinant amylase. C lane: supernatant of pelleted Pp03 culture (control); M lane: protein marker; AmyL lane: supernatant of pelleted Pp04 culture (amylase strain). The arrow on the right indicates the size of amylase (around 60 kDa) secreted to the media. (C) shows pre-lyophilization and post-lyophylization rates for amylase production.

FIG. 16 shows SEAP production in hydrogels outperforms tradition liquid suspension systems. SEAP production in hydrogels with repeated use compared to liquid culture performance. All the samples were treated with lyophilization after round 2 of reuse. Data are mean±s.d.; n=3 biological replicates.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION

Unless otherwise defined herein, scientific and technical terms used in connection with the invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art. The methods and techniques are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol. I, CRC Press (1976); Handbook of Biochemistry: Section A Proteins, Vol. II, CRC Press (1976); Essentials of Glycobiology, Cold Spring Harbor Laboratory Press (1999), which are incorporated herein by reference.

### Definitions

The following terms, unless otherwise indicated, shall be understood to have the following meanings:

The articles “a” and “an” are used herein to refer to one or to more than one (i.e., to at least one) of the grammatical object of the article. By way of example, “an element” means one element or more than one element.

The term “crosslink” refers to a bond or chain of atoms attached between and linking two different polymer chains.

The term “hydrogel” refers to a water-swellable polymeric matrix, consisting of a three-dimensional network of macromolecules held together by covalent crosslinks that can absorb a substantial amount of water to form an elastic gel.

The term “3D printed hydrogel” refers to a hydrogel that exists in 3 planes, so that it forms a three dimensional structure. Particles, referred to herein as loading agents or cells, may exist within the 3D printed hydrogel, and can be spatially arranged because of the multidimensional shape.

“Maintenance” of a cell or a population of cells refers to the condition in which a living cell or living cell population is neither increasing nor decreasing in total number of cells in a culture. Maintenance can be accomplished with or without production of a product from the cell population. Alternatively, “proliferation” of a cell or population of cells, as the term is used herein, refers to the condition in which the number of living cells increases as a function of time with respect to the original number of cells in the culture.

“Production” of cells refers to a product produced by the cells in the 3D printed hydrogel. For example, the cells can be in production when at least one product is being produced from the cells.

The term “cell,” as used herein, refers to both prokaryotic and eukaryotic cells. When referring to eukaryotic cells, the cells can be unicellular organisms, such as protozoa, algae, or fungi cells, or can be single cells from larger organisms, such as plants and animals. The cells can be mammalian cells, for example. The cells can also be prokaryotic cells, such as microorganisms like bacteria and archaea.

The term “population” or “cell population” as used herein refers to cells of the same type. For example, a population of a certain species of yeast can be present in a consortia, along with a different population of bacteria. When the term “cell population” is used herein, it refers to cells that share a common characteristic which distinguishes them from other cellular populations which also may be present, such as a certain species of unicellular organism or a certain mammalian cell type, such as CHO cells.

“Preservation” of the 3D printed hydrogel refers to the hydrogel being put under conditions under which the hydrogel can be stored, dehydrated, or maintained for extended periods of time. For example, preservation of the hydrogel can include lyophilization.

A “bioreactor” as defined herein refers to a composition producing a product. For example, the bioreactor can be the 3D printed hydrogel comprising the cellular organisms as disclosed herein. The bioreactor can be operated in batch, fed-batch, or continuous mode. The bioreactor can simply be a test tube comprising a hydrogel in contact with media, or can be much larger. The bioreactor can be monitored via computer.

In biological terms, a “community” or “consortia” is a group of coexisting organisms sharing an environment. The community can include one or more populations of cells, such as unicellular organisms like bacteria or yeast. For example, a community can include two different populations. A “microbial community” or “microbial consortia” is a community of microorganisms, i.e. microbes.

In biological terms, “culture” is the act or process of cultivating living material (such as unicellular organisms) in prepared nutrient media; culture also refers to a product of such cultivation. Culturing of microorganisms may be performed by inoculating a known concentration of microorganisms into a solution (culture medium typically containing nutrient media; optionally nutrient media that is modified, that contains desired additives, etc.) The cells can be cultured within the hydrogels disclosed herein.

“Culture medium” (also called “nutrient medium”, “growth medium”, and “medium”) refers to a substance, either solid or liquid, used for the incubation, cultivation, isolation, identification, or storage of microorganisms. Culture medium may include various components such as nutrients and optionally a variety of additives, including minerals, vitamins, amino acids, peptides, hormones, cell culture extracts of unknown composition, cell lysates of unknown composition, etc. The composition of the culture medium may differ when different types of microorganisms are cultured. The culture medium may optionally be modified (e.g. some compounds may be omitted from the culture medium when one wants to starve the microorganisms, or one wants to apply selection pressure). The culture media can be present in the hydrogels disclosed herein, or can be added to the hydrogel before, during, or after the addition of cells.

“Co-incubation” of microorganisms refers to joint incubation or incubation together, of two or more types (e.g. organisms, populations, strains, species, genera, families, etc.) of microorganisms. In the context of the present invention, co-incubation of microorganisms refers to the joint incubation of two or more types of microorganisms in a microbial community. Co-incubation of microorganisms is also meant to include co-culture (i.e., joint culture, or culture together) of microorganisms, but growth is not required for co-incubation.

### Platforms and Compositions

Described herein is a 3D printed hydrogel-based system for harnessing the bioactivity of embedded microbes for on-demand small molecule and peptide production in mono-culture and microbial consortia systems. This platform bypasses the challenges of multi-organism consortia by spatially organizing organisms into hydrogel constructs that precisely control the final consortium composition and dynamics without the need for synthetic control. Furthermore, these hydrogels can provide protection from preservation techniques (including lyophilization) and can sustain active metabolic function for long term, repeated use. Of note, once cell growth and proliferation has occurred within the hydrogel, product yields can be increased as the sugar does not need to go to biomass. This is the case with subsequent rounds of use of these hydrogels as well. The utility of this approach for the production of multiple, different chemical compounds, a peptide antibiotic, and carbohydrate catabolism by using either mono-cultures or co-cultures consisting of cross-feeding or parallel consortia has been shown by way of example (Example 1), although the methods and compositions disclosed herein are not limited by these examples, as they serve as exemplary illustrations. The printed microbe-laden hydrogel constructs' efficiency in repeated production phases, both pre- and post-preservation, outperforms liquid culture.

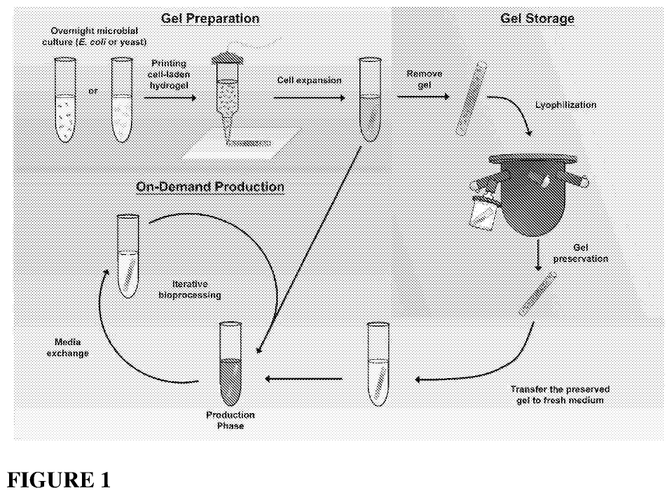

Disclosed herein is a platform that can spatially organize individual microbes and consortium members via direct-write 3D printing of microbe-laden hydrogel inks. It is demonstrated herein that mono- and co-culture systems can be 3D printed to form solid-state bioreactors capable of producing small molecules and antimicrobial peptides for multiple, repeated cycles of use. The structures 3D printed with these inks can be preserved via lyophilization, stored in a dried state, and re-hydrated at a later time for on-demand chemical and pharmaceutical production (FIG. 1) in a manner that outperforms a traditional liquid-based culture format. For co-cultures, these 3D constructs can spatially compartmentalize microbes to enable precise control of consortium composition and dynamics without the need for genetically encoded mutualism.

In the 3D printed hydrogels disclosed herein, at least two different populations of cells can be embedded in the 3D printed hydrogel. The cells can comprise eukaryotes, prokaryotes, or a combination of both. The cells can comprise any type of cell, such as eukaryotic cells such as mammalian cells, yeast, or fungi, as well as prokaryotic cells such as bacteria or archaea, or a combination of two or more of these. At least one of the populations of cells can produce at least one product. The 3D printed hydrogel can comprise 1, 2, 3, 4, 5, 6, 7, 8, 9, 10, or more different populations of cells. When more than one cell population is present, the two or more populations can be from different kingdoms, different phyla, different families, different genera, different species, or different subspecies. In microbial communities, resources, preferences, and a number of other conditions may be present and common, affecting the identity of the population and their degree of cohesiveness.

Importantly, it was discovered that more than one population of cells can not only survive in the same 3D printed hydrogel, but thrive in such an environment. Both the longevity of the cells over repeated use cycles, as well as the production of product by the cells, was different than what would have been expected by one of skill in the art. Furthermore, not only the survival of these organisms, but the continued high levels of production after preservation was remarkable. In the examples disclosed herein, the production levels for some of the consortia after preservation were on par with production levels before preservation. Also remarkable is that these cells continued surviving and producing at high levels even without the addition of chemical protectants such as glycerol and/or sorbitol.

At least one of the populations of cells present in the 3D printed hydrogel can produce at least one product. For example, two different species of microorganisms can be present, and one can produce a product, while the other does not. Alternatively, both populations of microorganisms can produce a product. Interactions among various populations may be competitive, they may be mutually beneficial, or they may be mutually harmful (e.g., via production of toxic products, competition for nutrients, etc.), as long as the interactions together can provide a function or a structure that is not available through either population. The compositions and methods of the present invention provide one or more populations with conditions that allow those members to perform certain function while reducing accompanying undesirable or harmful effects. One population may produce a product, while another consumes it. In another example, more than one population may produce a product, and those products may combine to form a third product. In one example, the different populations of microorganisms can be in a symbiotic relationship.

At least one population of cells that are embedded in the 3D printed hydrogel can consistently produce a product for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 90, 120, 180, 210, 240, 270, 300, 330, 360, or more days while embedded in the 3D printed hydrogel disclosed herein.

The disclosed compositions and methods can provides for conditions that enable culturing of cells that in nature prefer mutually less compatible, incompatible, or exclusive conditions, such as different partial concentration of oxygen, different pH, different composition of nutrients, and different growth rates of the cells that can lead to out-competition. The cultivation of some microbial communities may require externally controlled environments that can be provided, such as temperature. Some populations may distinguish themselves by producing, consuming, modifying, degrading, and/or accumulating particular compounds. There are also microbial populations that can, over time, create preferred different environments (e.g. aerobic, anaerobic, facultative anaerobic, etc.), again by producing, consuming, modifying, degrading, and/or accumulating particular compounds. The practice of the present invention specifically contemplates all of the above types of microbial communities.

An important concept of the present invention is the ability to maintain separated populations in the hydrogel. By “separated” is meant generally located in different physical locations, although the cell populations do not have to be completely isolated from each other in order to be considered “separated.” For example, cells from one population can “move” or “drift” or “diffuse” into another area of the hydrogel, and may come into contact with another population. The cells are still considered “separated” if at least 50, 60, 70, 80, or 90% or more of the cells remain in a generally separate area. Furthermore, the cell populations can be separated by a biofilm for example.

If cells are too close (mixed together such that there is no spatial structure, physically touching, or not immobilized) then they will directly compete (either through competition for shared nutrients, one organism releasing waste or a chemical that inhibits the growth or activity of another organism, or different growth rates). By physically separating the cells over space, competition is reduced and macroscopic carrying capacity for these populations is controlled by altering the respective gel volume. However, communities' functions may involve the exchange of chemical between different cells. In that case, the cells should be separated to reduce competition, they also should be close enough such that they can effectively exchange chemicals. Effective intercellular communication distances can also be determined by methods such as the relative time constants for secretion and diffusion of molecules (Francis 1997).

The 3D printed hydrogels disclosed herein can be preserved by a variety of methods. Any method recognized in the art for preserving a hydrogel can be used to provide a preserved hydrogel matrix according to the present invention. For example, the hydrogels comprising the cells can be dehydrated, freeze dried (lyophilized), stored in a refrigerator, or cryopreserved using any method known to those of skill in the art.

Importantly, the cells can withstand the preservation process and continue to survive and produce product.

In one embodiment, the hydrogel matrix is dehydrated, such as by lyophilization. A lyophilized hydrogel matrix is similarly disclosed in U.S. Patent Application Publication 2005/0118230, incorporated herein by reference in its entirety. One method of preserving the hydrogel matrix is freeze drying. Other methods of preparing dehydrated biopolymers, such as spray-drying or speed-vac, can also be used and are known to those skilled in the art.

Lyophilizing, or freeze-drying, generally comprises the removal of water or other solvent from a frozen product through sublimation, which is the direct transition of a material (e.g., water) from a solid state to a gaseous state without passing through the liquid phase. Freeze drying allows for the preparation of a stable product being readily re-hydratable, easy to use, and aesthetic in appearance.

Also disclosed herein is a composition comprising a lyophilized and rehydrated 3D printed hydrogel, wherein at least one population of cells are embedded in the 3D printed hydrogel, and wherein the at least one population of cells is capable of producing at least one product; and further wherein the at least one product is capable of being produced at a rate of 50% or higher compared to production of the same product produced from an identical population of cells, present in substantially similar numbers, in an identical 3D printed hydrogel, wherein the cells in the identical 3D printed hydrogel are not lyophilized or rehydrated.

One example of equipment useful in preparing freeze dried hydrogels is the FreeZone 12 Liter Freeze Dry System with Stoppering Tray Dryer (Labconco, Kansas City, Mo.). With such system, tubes with porous caps containing hydrogels are frozen to −30° C. at a cooling rate of 0.05° C./min using the cooling shelf unit of the freeze dryer and are held at −30° C. for 24 hours. After lyophilization or preservation and subsequent rehydration, at least one product can continue to be produced from at least one population of bacteria within the hydrogel. For example, the product can be produced at a rate of 5, 10, 15, 20, 25, 30, 35, 40, 45, 50, 55, 60, 65, 70, 75, 80, 85, 90, 95, or 100% as compared to production of at least one product prior to lyophilization and rehydration of the composition. This level of production can occur for at least 1 2, 3, 4, 5, 6, 7, 8, 9, 10, 11, or 12 months or more after lyophilization and rehydration.

More than one cycle of lyophilization and rehydration can occur. For example, the 3D printed hydrogel can be subjected to 2, 3, 4, 5, 6, 7, 8, 9, 10, or more cycles of lyophilization and rehydration. High production rates can continue from at least one population of bacteria within the 3D printed hydrogel, even after more than one cycle of lyophilization and rehydration. For example, the product can be produced at a rate of 5, 10, 15, 20, 25, 30, 35, 40, 45, 50, 55, 60, 65, 70, 75, 80, 85, 90, 95, or 100% after two rounds of lyophilization and rehydration. This level of production can occur for at least 1 2, 3, 4, 5, 6, 7, 8, 9, 10, 11, or 12 months or more after two or more rounds of lyophilization and rehydration.

When comparing production of a product from a population of cells in a lyophilized, rehydrated 3D printed hydrogel, it is understood that what is meant by “identical” is that all the conditions are substantially the same when comparing the amount of product produced from a population of cells in a lyophilized and rehydrated hydrogel, as compared to the amount of product produced from a population of cells in a hydrogel which has not been lyophilized or rehydrated. The conditions can be the same such as temperature, pH, stability, exposure to other chemicals, etc. The population of cells can be made up of identical, or nearly identical cell populations, and can be present in the same or substantially the same numbers. The cells can be organized within the 3D printed hydrogel in substantially the same way, such as the same spatial arrangement or distance from each other. And the 3D hydrogels can be comprised of the same or substantially the same material, as described elsewhere herein.

The cells can be spatially organized in the 3D printed hydrogel. For example, at least two different species can occupy at least two different spatial areas in the hydrogel. In one example, to inoculate the hydrogels, the hydrogel solutions can be lowered in temperature to about 4° C. in a refrigerator. At this temperature, the hydrogel solutions undergo a gel-to-sol transition, affording a low-viscosity liquid. The cells and photoradical generator can be added to this liquid, which is then warmed to ambient temperature to reconstitute the shear-thinning hydrogel. 3D printing is performed using a multi-nozzle extrusion-based 3D printer. Each nozzle of the printer can deposit a separate shear-thinning hydrogel comprised of cells. A computer-aided design (CAD) model which represents the digital form of the desired spatial organization of the cell-laden hydrogels can be created, and determines the structure and spatial organization of the 3D printed hydrogel. The CAD model can then be transformed into G-code commands that are used by the printer to construct the 3D printed hydrogel.

A variety of hydrogels may be used according to the present invention. Examples of some hydrogels that can be used with the invention disclosed herein follow.

As used herein, the term “alkyl” refers to a straight or branched, saturated, aliphatic radical having the number of carbon atoms indicated. For example, C₁-C₆ alkyl includes, but is not limited to, methyl, ethyl, propyl, isopropyl, butyl, isobutyl, sec-butyl, tert-butyl, pentyl, isopentyl, hexyl, etc. The alkyl group is typically monovalent, but can be divalent, such as when the alkyl group links two moieties together.

As used herein, the term “alkenyl” refers to either a straight chain or branched hydrocarbon of 2 to 6 carbon atoms, having at least one double bond. Examples of alkenyl groups include, but are not limited to, vinyl, allyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

The 3D hydrogel can comprise a polymer of Formula (I):

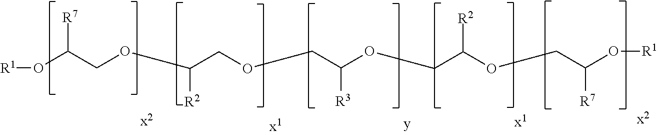

wherein
R¹ is hydrogen or a group having the formula:

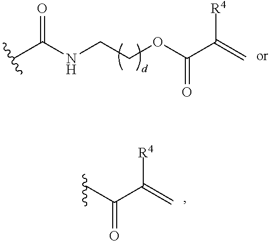

wherein d is 1, 2, 3, 4, 5, 6, 7, 8, 9, 10, 11, or 12; and R⁴ is hydrogen or methyl;
R² is hydrogen or —CH₂OR⁵; wherein R⁵ is C₁₋₁₂alkyl, C₁₋₁₂alkyl-OR⁶ or C₁₋₁₂alkyl-NR⁶₂, wherein each R⁶ is independently hydrogen or C₁₋₁₂alkyl;
R³ is hydrogen or methyl; and
R⁴ is hydrogen or methyl;
wherein R⁷ is —CH₂—O—C₁₋₁₂alkenyl;
y is selected to provide a block polymer with an Mₙ of about 500 to about 50,000;
x¹ and x² are selected to provide a block polymer with an Mₙ of about 100 to about 30,000;
x¹ is from 10-100; and y is from 25-250, provided that y is greater than x¹.

The 3D hydrogel can also comprise a polymer having the structure of Formula (II):

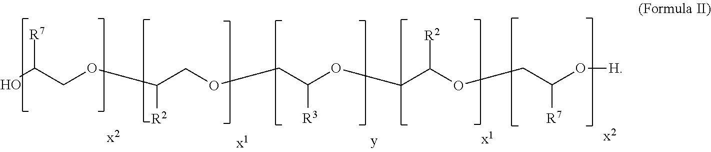

R² can be —CH₂—O—C₁₋₆alkyl; and R⁷ can be —CH₂—O—C₂₋₆alkenyl.

The polymer can have the structure of Formula (IIa):

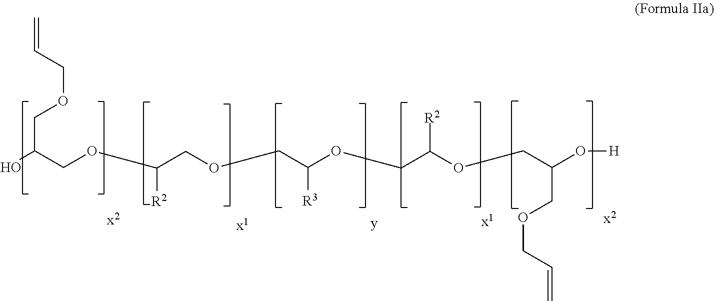

R² can be —CH₂—O—C₁₋₆alkyl.

The polymer can have the structure of Formula (IIb):

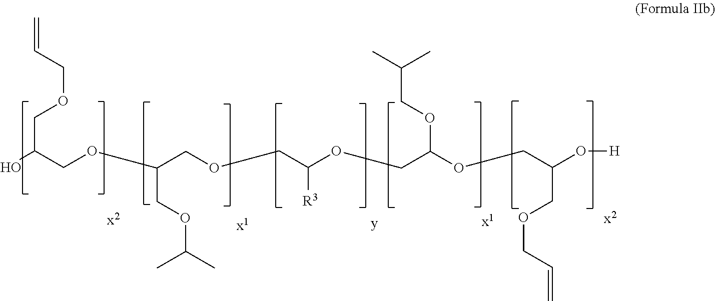

The polymer can have the structure of Formula (IIc):

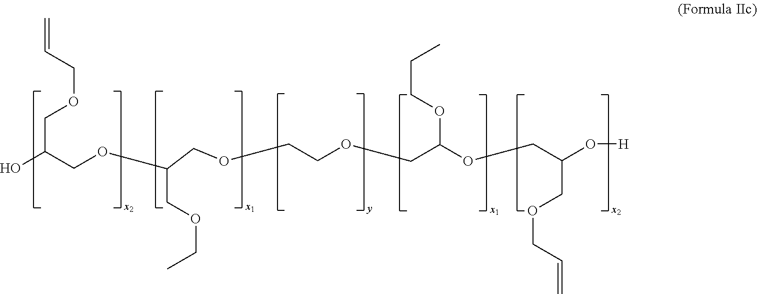

The polymer can have the structure of Formula (III):

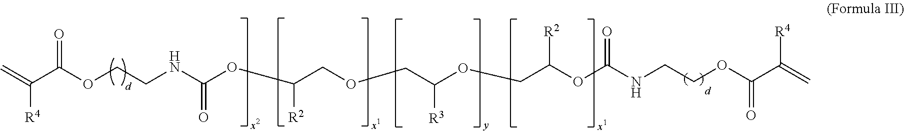

R² can be —CH₂—O—C₁₋₆alkyl.
The composition can have the structure of Formula (IIIa):

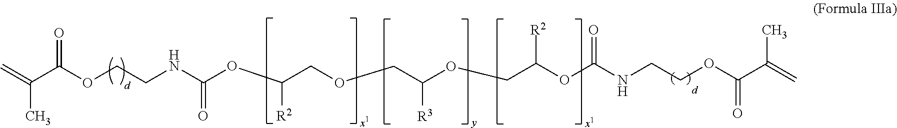

The polymer can have the structure of Formula (IIIb):

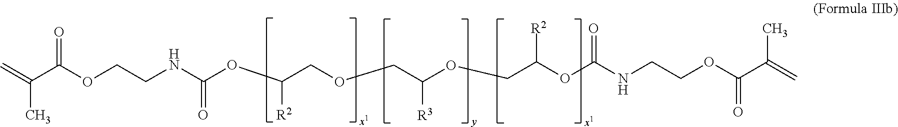

The polymer can have the structure of Formula (IIIc):

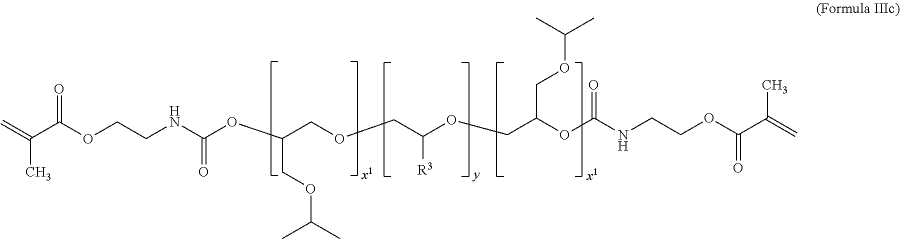

The composition comprising a 3D printed hydrogel, as disclosed herein, may be a triple stimuli-responsive hydrogel, which is responsive to temperature, shear stress and light. A significant challenge for thermoresponsive and stress responsive hydrogels (e.g., F127 and iPGE based hydrogels) is that the after printing, the printed structures continue to respond to stimuli. This results in unstable printed materials, which can degrade when exposed to cold temperature or if a force is applied.

The temperature response of the hydrogel composition is important for creating homogeneous gels. At about 25° C., the hydrogel may be firm and capable of holding its form. At about 10° C., the same hydrogel may become a viscous fluid, which enables delicate materials like living cells to be incorporated in to the mixture with gentle stirring. When the mixture is warmed to ambient temperature, it becomes a gel again. This gentle method of forming a homogeneous hydrogel mixture is advantageous compared to other approaches for hydrogel formation, which include, for example, elevated temperatures, sonication, or vortexing, all of which can potentially be detrimental to delicate cells.

The response of the hydrogel composition to applied shear stress is important for extrusion printing of the composition. A shear-thinning hydrogel is ideal for this type of patterning, and can enable the formation of three-dimensional lattice structures comprised of the cell-laden hydrogel.

The response of the hydrogel composition to light is important to initiate crosslinking of the hydrogel composition to form a crosslinked hydrogel structure. The polymer hydrogel may be designed so that when it is exposed to light at particular wavelength (e.g., 365 nm), the polymer undergoes a chemical reaction that covalently crosslinks the polymer chains together. This crosslinked material is “fixed,” and cannot re-dissolve when placed in water, nor exhibit temperature-responsive behavior.

In some embodiments the hydrogel composition may include a polymer of Formula (I) and an aqueous media. The polymer may be present in an amount between about 10 weight percent and about 50 weight percent of the hydrogel composition. In other embodiments, the polymer may be present in the hydrogel composition in an amount between about 10 weight percent and about 45 weight percent, about 10 weight percent and about 40 weight percent, about 10 weight percent and about 35 weight percent, about 10 weight percent and about 30 weight percent, about 10 weight percent and about 25 weight percent, about 10 weight percent and about 20 weight percent, about 10 weight percent and about 15 weight percent, about 15 weight percent and about 45 weight percent, about 15 weight percent and about 40 weight percent, about 15 weight percent and about 35 weight percent, about 15 weight percent and about 30 weight percent, about 15 weight percent and about 25 weight percent, about 15 weight percent and about 20 weight percent, about 10 weight percent and about 20 weight percent, about 15 weight percent and about 25 weight percent or about 15 weight percent and about 20 weight percent. In other embodiments, the polymer may be present in the hydrogel composition in an amount between about 12 weight percent and about 22 weight percent, about 12 weight percent and about 18 weight percent, about 14 weight percent and about 22 weight percent or about 14 weight percent and about 18 weight percent of the hydrogel composition.

The aqueous media of the polymer composition may be any liquid capable of solubilizing the polymer of Formula (I). In some embodiments, the aqueous media is water, an aqueous buffer or saline. The aqueous may be selected to be compatible with the loading agent of the hydrogel composition and with the chemical process that the crosslinked hydrogel structure will be used to catalyze. For example, when the loading agent is a marine organism or the chemical process is to occur in saltwater, the aqueous media may be saline. Other examples of aqueous media includes synthetic complete (SC), yeast extract peptone dextrose (YPD), lysogeny broth (LB), RACl, COREs, Guillard's F/2, and M9, MOPS, and rich media (RM). Also contemplated is the use of mammalian cell media.

In some embodiments, the hydrogel composition may form a shear-thinning gel between about 18 and about 45° C. In other embodiments, the hydrogel composition forms a shear-thinning gel between about 20 and about 30° C., between about 18 and about 25° C., between about 20 and about 25° C., between about 18 and about 22° C., between about 22 and about 30° C., or between about 22 and about 28° C.

In some embodiments the hydrogel composition may further include a photoinitiator. The photoinitiator can be any molecule known in the art to produce a radical species when exposed to a certain wavelength of light. For example, the initiator may be a peroxide, azobisisobutyronitrile or 2-hydroxy-2-methylpropiophenone. Other photoinitiators include benzophenone, ethyl 2,4,6-trimethylbenzoylphenyl phosphinate, 1-hydroxy-cyclohexyl-phenyl-ketone, 2-hydroxy-2-methyl-1-phenyl-1-propanone, 2,2-dimethoxy-2-phenyl acetophenone, 2-hydroxy-1-[4-(2-hydroxyethoxy)phenyl]-2-methyl-1-propanone, and diphenyl (2,4,6-trimethylbenzoyl)-phosphine oxide.

The photoinitiator is present in an amount between about 0 weight percent and about 20 weight percent of the polymer. In some embodiments, the photoinitiator is present in an amount between about 1 weight percent and about 20 weight percent, 1 weight percent and about 15 weight percent, 1 weight percent and about 10 weight percent, 1 weight percent and about 5 weight percent, 3 weight percent and about 20 weight percent, 3 weight percent and about 15 weight percent, 3 weight percent and about 10 weight percent, 3 weight percent and about 5 weight percent, 5 weight percent and about 20 weight percent, 5 weight percent and about 15 weight percent, or 5 weight percent and about 10 weight percent of the polymer In some embodiments the hydrogel composition may further include a loading agent.

The loading agent can be any material, soluble or insoluble that can be mixed with the hydrogel composition prior to extrusion printing. In the present invention, an example of a loading agent is cells. As described herein, more than one population of cells can be embedded in the 3D printed hydrogel. In addition to cells, other loading agents can be mixed with the hydrogel prior to extrusion or printing. This can include, but is not limited to, a pharmaceutical drug, a hydrophobic additive, an organic or inorganic chemical substance, a catalyst, nanomaterials such as nanoparticles, nanotubes, and graphene, a polymer, a biopolymer, or a living cell.

In another aspect, the disclosure provides a crosslinked hydrogel structure including a polymer of Formula (I), an aqueous media, one or more populations of cells, and optionally a further loading agent, wherein the polymer includes crosslinks derived from the alkene groups of R2 in different polymer chains, or the polymer includes crosslinks derived from the alkene groups of the (meth)acrylate derivative in different polymer chains. Also disclosed is a method for forming a crosslinked hydrogel structure. The method may involve subjecting a polymer composition including a polymer of Formula (I), an aqueous media, a photoinitiator and a loading agent to UV light to initiate crosslinking between and the alkene groups of R2 in different polymer chains, or between the alkene groups of the (meth)acrylate derivative in different polymer chains.

When the polymer of the hydrogel composition have the structure of any of Formula (II), (IIa), (IIb) or (IIc), the crosslinks of the crosslinked hydrogel structure are derived from the alkene groups of R2 in different polymer chains. That is, the crosslinked hydrogel structure is formed by subjecting a hydrogel composition including a polymer of Formula (II), (IIa), (IIb) or (IIc) to crosslinking conditions, optionally in the presence of a photoinitiator, to crosslink the alkene groups of R2 in different polymer chains.

Similarly, when the polymer of the hydrogel composition have the structure of any of Formula (III) or (IIIa)-(IIIc), the crosslinks of the crosslinked hydrogel structure are derived from the alkene groups of (meth)acrylate derivative in different polymer chains. That is, the crosslinked hydrogel structure is formed by subjecting a hydrogel composition including a polymer of Formula (III) or (IIIa)-(IIIc) to crosslinking conditions, optionally in the presence of a photoinitiator, to crosslink the alkene groups of (meth)acrylate derivative in different polymer chains.

In some embodiments, the crosslinking is performed on the extruded hydrogel composition. In other embodiments, the crosslinking is performed on the hydrogel composition directly (i.e., without extrusion printing).

Crosslinking of the hydrogel composition to provide a crosslinked hydrogel structure significantly changes the properties of the hydrogel composition. Following crosslinking, the crosslinked hydrogel structure has improved mechanical robustness and viscoelastic properties, is not soluble in aqueous solution and maintains transparency.

For hydrogel composition including the polymer of Formula (III) crosslinking occurs between the methacrylate groups, and the urethane moieties offer hydrogen bonding motifs that can help preserve elasticity and mechanical robustness of printed structures after crosslinking.

In some embodiments, the hydrogel composition further includes a photoinitiator. In other embodiments, the hydrogel composition further includes a loading agent. In some embodiments, the hydrogel composition further includes a photoinitiator and cells.

In some embodiments, the hydrogel composition is printed into a multi-layer patterned structure. The hydrogel composition can be transferred into the printer syringe by cooling the hydrogel composition to provide a liquid and pouring the solution. Upon warming to ambient temperature, the hydrogel composition can become a gel that can be printed via extrusion to provide an extruded hydrogel composition. The thickness of the extruded hydrogel composition can be controlled by modifying the nozzle diameter, as well as the print speed. For example, the nozzle can be a 210, 260, or 410 m diameter. As the speed at which the nozzle moves across the printing surface is increased, the diameter of the printed strand is decreased. The extruded hydrogel composition can be printed in a grid of up to 40 layers demonstrating without the lines supporting the structure buckling or sagging.

The temperature at which the extrusion printing is performed will depend on the properties of the hydrogel composition being printed. In some examples, the extruded hydrogel composition is a gel at ambient temperature, so the extrusion printing may be performed at ambient temperature. Thus, the temperature can range from 10° C. to 45° C. In other embodiments, the hydrogel composition is cooled below ambient temperature to provide the hydrogel composition as a liquid for homogenously incorporating the loading agent.

The method may also further involve subjecting the extruded hydrogel composition to crosslinking conditions to provide a crosslinked hydrogel structure. For example, UV light may be used to initiate crosslinking between the alkene groups of R2 in different polymer chains, or between the alkene groups of the (meth)acrylate derivative in different polymer chains to form a crosslinked hydrogel structure.

Crosslinking of the extruded hydrogel composition will introduce a permanently crosslinked network of polymer chains and hence will produce a mechanically robust hydrogel structure. This will broaden the scope of the hydrogel structures for various applications in aqueous media such as in chemical catalysis, production of antibiotics, bio fuels cells, waste water treatment, etc. The crosslinked hydrogel structure enables the use of 3D printed objects in such applications where traditional 3D printed hydrogels are prone to degradation.

The extruded hydrogel composition can be cured for about 1 second to about 10 minutes by exposure to irradiation. The irradiation wavelength is in the range of 100 nm to 700 nm. In some embodiments, the irradiation wavelength is in the range of 200 nm to 700 nm, 100 nm to 650 nm, 200 nm to 700 nm, 200 nm to 650 nm, 300 nm to 700 nm, 350 nm to 700 nm, 400 nm to 700 nm, 450 nm to 700 nm or 500 to 700 nm. In other embodiments, the irradiation wavelength is in the range of 200 nm to 600 nm, 100 nm to 550 nm, 200 nm to 600 nm, 200 nm to 550 nm, 300 nm to 600 nm, 350 nm to 600 nm, 400 nm to 600 nm, 450 nm to 600 nm or 500 to 600 nm.

In some embodiments, the curing is performed in a UV cure box having 365 nm wavelength irradiation with a power of 3.4 mW/cm². Crosslinking allows for robust hydrogel structures to be held with forceps without damaging the structure.

Also disclosed is a bioreactor comprising any of the compositions disclosed herein. A bioreactor is a generalized term that essentially covers any kind of vessel that is capable of incubating cells while providing a degree of protection for the cells' environment. This can include simple batch processes, such as those carried out in a tube. As used herein, the 3D printed hydrogel with embedded microorganisms can be placed in any type of suitable vessel or container, or under conditions which promote production of a desired product from cells. The conditions of the bioreactor may not be fully controlled and monitored. On the other hand there are fully automated electromechanical state-of-the-art bioreactors in which all the variables are monitored and controllable. Many inter-combinations between these examples are well known to one of ordinary skill in cellular biotechnology.

### Methods of Producing a Product

In one aspect, the invention provides methods of producing a product from cells embedded within a 3D printed hydrogel. The method can comprise providing a composition comprising a 3D printed hydrogel wherein at least two different populations of cells are embedded in the 3D printed hydrogel, and further wherein at least one of the populations of cells produces a product; and exposing the composition to conditions favorable to produce the product.

As discussed herein, at least two different populations of cells can be embedded in the hydrogel produce a product. The products of these different organisms can be the same or different. In one embodiment, a first population of cells produces a product relied upon by the second population of cells.

Conditions of the composition disclosed herein can be adjusted to maximize production of at least one product produced by at least one population of cells in the composition. At least one of the populations of cells can produce a product for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more consecutive days. The composition can undergo lyophilization and reconstitution prior to producing a product. At least one of these products is capable of being produced at a rate of 50% or higher after lyophilization and rehydration of the composition as compared to production of at least one product prior to lyophilization and rehydration of the composition. In another embodiment, at least one product is capable of being produced at a rate of 50% or higher after lyophilization and rehydration of the composition at least a year or more after lyophilization and rehydration. For example, at least one product is capable of being produced at a rate of 50% or higher after more than one lyophilization and rehydration cycle.

Further disclosed is a method of producing a composition comprising a 3D printed hydrogel, wherein the 3D printed hydrogel comprises at least two different populations of cells; the method comprising: providing a shear-thinning hydrogel; embedding in said hydrogel at least two different populations of cells; 3D printing said hydrogel comprising at least two different populations of cells onto a suitable substrate, thereby providing a composition comprising a 3D printed hydrogel. In one embodiment, the cells can be expanded by growing them. In another embodiment, the composition can be lyophilized and reused.

In the methods disclosed herein, the 3D printed hydrogel can be cross-linked. At least two different populations of cells can form a consortia. At least two different populations of cells can be embedded in at least two separate shear-thinning hydrogels. Interactive dynamics between the at least two different populations of cells embedded in the at least two separate shear-thinning hydrogels can be controlled. For example, interactive dynamics can be controlled by printing the two separate hydrogels in different amounts. Interactive dynamics can be controlled by controlling amounts of individual cells embedded in each liquid state hydrogel. Interactive dynamics can be controlled by controlling conditions under which the composition is maintained. These conditions can comprise temperature and cell media.

The composition comprising the 3D printed hydrogel can be preserved. In one embodiment, the composition is preserved by lyophilization and subsequently reconstituted. After lyophilization and rehydration, at least one product is capable of being produced at a rate of 50% or higher as compared to production of at least one product prior to lyophilization and reconstitution of the composition. At least one product is capable of being produced at a rate of 50% or higher after lyophilization and reconstitution of the composition at least a year or more after lyophilization and reconstitution. At least one product is capable of being produced at a rate of 50% or higher after more than one lyophilization and reconstitution cycle.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1: 3D Printed Microbe and Consortia Hydrogels for on Demand Production and Bioprocess Preservation

To test the repeated use and preservation capacity of these microbial-laden hydrogel inks, a series of demonstration tests using mono-cultures were done to produce a variety of important biochemical compounds and an antimicrobial agent. In each case, the microbe-laden hydrogel was based on 30 wt % F127-BUM, a polymer that forms a temperature responsive and shear-thinning hydrogel capable of encapsulating yeast (e.g., Saccharomyces cerevisiae) and/or bacterial (e.g., Escherichia coli) cells prior to forming a robust, elastic polymer network after UV-initiated crosslinking. For these studies, mono-culture hydrogels were evaluated for continuous production over multiple fermentation cycles, where the initial cell outgrowth stage was defined as round 0 of gel use and the repeated, on-demand production phase (either immediately after outgrowth or post-preservation) as round 1 and onward (FIG. 1).

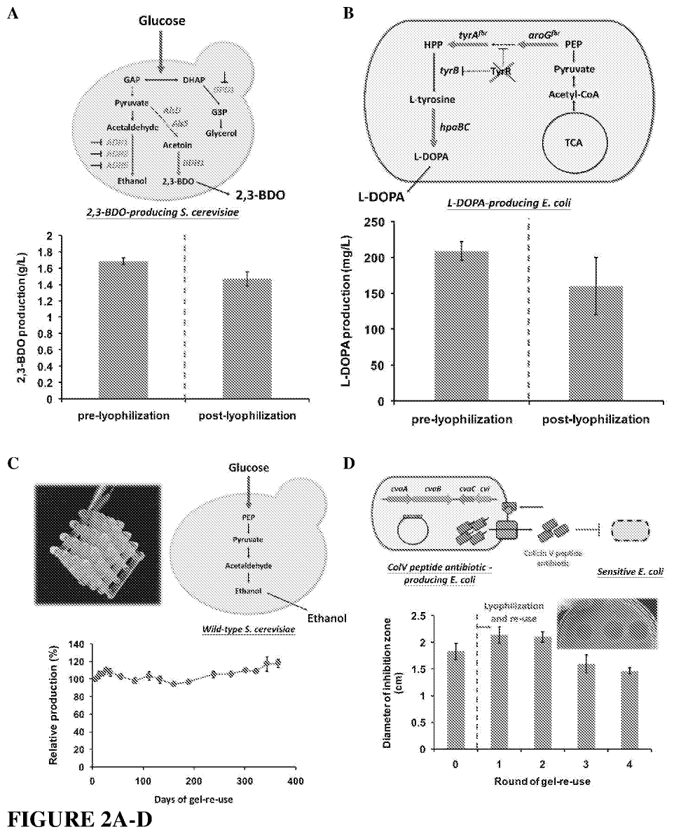

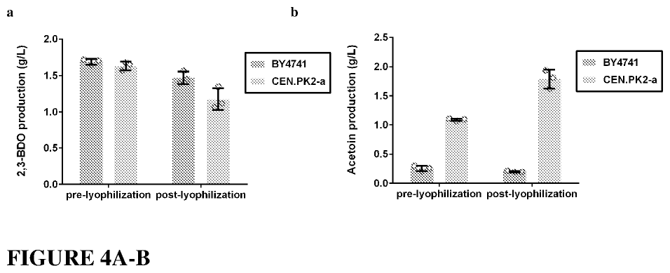

As test cases, the production of value-added biochemicals, 2,3-butanediol (2,3-BDO) and 3,4-dihydroxy-L-phenylalanine (L-DOPA) were tested as examples, due to their market potential and their ability to evaluate two distinct metabolically engineered microbial hosts in these printed constructs (Köpke 2017). First, a yeast-strain overproducing 2,3-BDO (Deaner 2018) was encapsulated in the gel matrix and production (titer at 48 hours in a test tube) was assessed both pre- and post-preservation (FIG. 2A). It should be noted that the lyophilization procedure included a 10-minute freezing step and vacuum drying without the use of any cryoprotectants. Nevertheless, after lyophilization, the preserved yeast-laden hydrogel retained nearly 90% of its BDO production capacity (1.5 g/L) compared to a paired sample prior to preservation (1.7 g/L). While these initial results were obtained for an S. cerevisiae BY4741-based strain, a similar phenomenon was also observed with an alternative strain constructed in the CEN.PK2 background (FIG. 4).

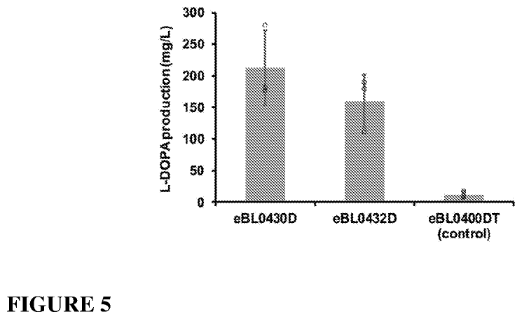

Second, the performance of the hydrogel using a metabolically engineered strain of E. coli designed to overproduce L-DOPA (FIGS. 2B and 5) was evaluated. In similar fashion to the 2,3-BDO yeast example, L-DOPA production from printed hydrogels did not differ significantly (p>0.05) pre- and post-lyophilization (both producing >150 mg/L after 22 hours). Collectively, these results demonstrate that yeast and bacteria-laden gels were both metabolically active, and that entrapment of cells in the F127-BUM hydrogel resulted in minimal loss of biocatalytic activity during lyophilization.

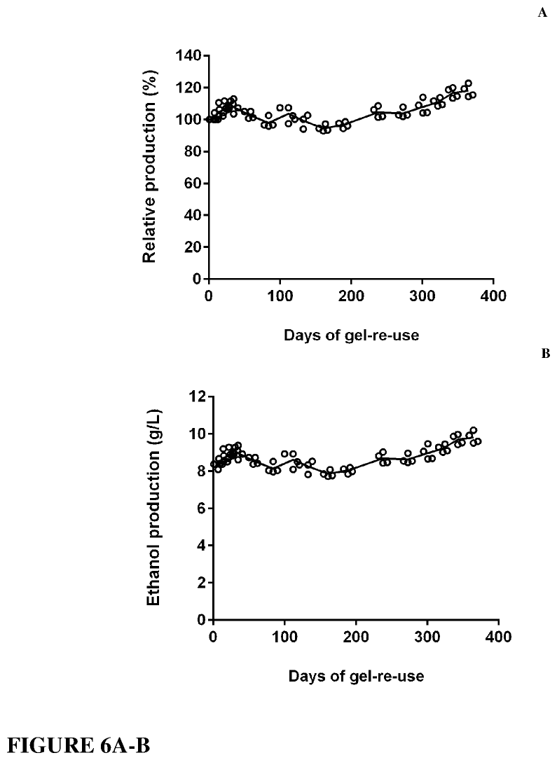

Third, the long-term stability and re-use potential of the cell-laden gels for continued, repeated production was evaluated. To do so, a yeast-laden hydrogel lattice (containing S. cerevisiae SO992) was printed and its ethanol production capacity evaluated over the course of one year of repeated batch culturing (FIG. 2C). Through this one-year period, the average titer achieved in these batches (8.74±0.53 g/L) exhibited no noticeable decrease in performance and, if anything, slightly increased throughout the process (FIG. 6). It should be noted that all four replicate samples were still operational at the end of the year-long process, which demonstrated the robustness and potential longevity-enhancing capacity of these hydrogel materials.

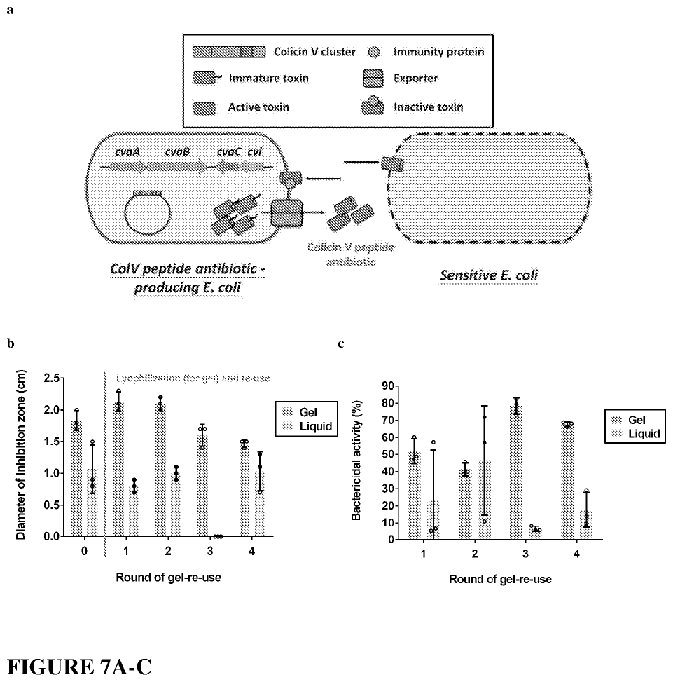

Finally, one additional mono-culture within the microbe-laden hydrogel constructs was evaluated that demonstrated the ability of larger molecules (in this case, the peptide antibiotic, colicin V) to be produced and effectively diffuse out of the gel and into the surrounding culture media. To do so, gels containing E. coli capable of secreting the pore-forming antibiotic colicin V (ColV) were printed (Gilson 1987) (FIG. 2D). For both the round 0 gel use and for four consecutive uses after lyophilization, the E. coli-laden hydrogel was able to produce colicin V at relatively constant levels as measured by a zone-of-inhibition assay. Furthermore, this gel-based performance was compared to that of a traditional free-cell system culture process (FIG. 7B). During this comparison, the level of ColV antimicrobial peptide produced by a free cell system was inconsistent across iterative subculture cycles, unlike the microbe-laden hydrogel that retained more consistent production across repeated uses. Specifically, no zone-of-inhibition was observed for the free cell system by the third round of re-use, and the bactericidal activity of the corresponding supernatant was dramatically reduced to 6.5% of its prior value. Collectively, these mono-culture results demonstrate that this hydrogel ink formulation can enable reusable and stable, on-demand production of both small molecules and peptide antibiotics using yeast or bacteria.

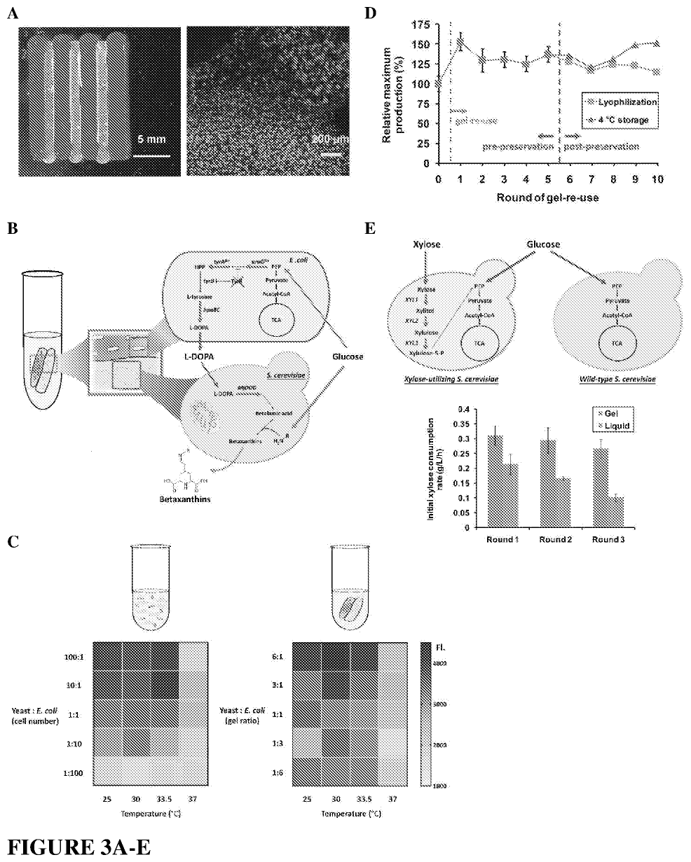

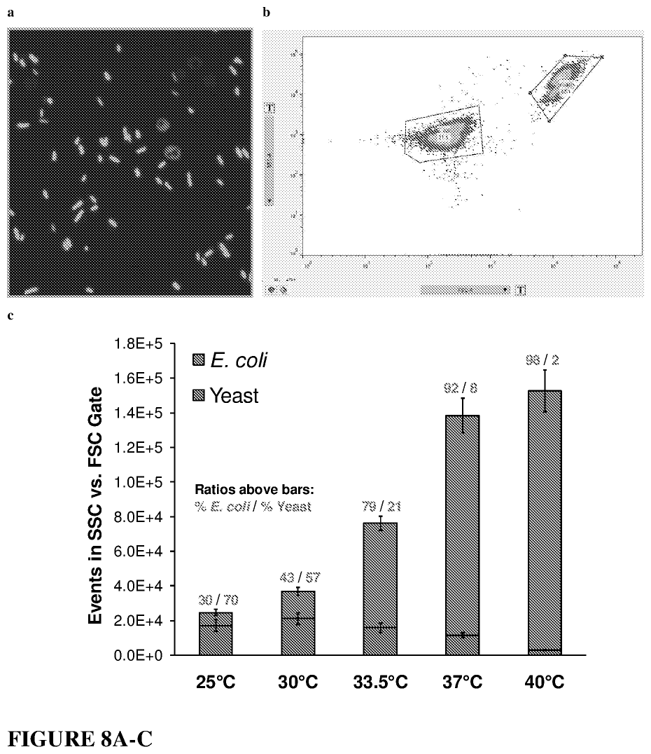

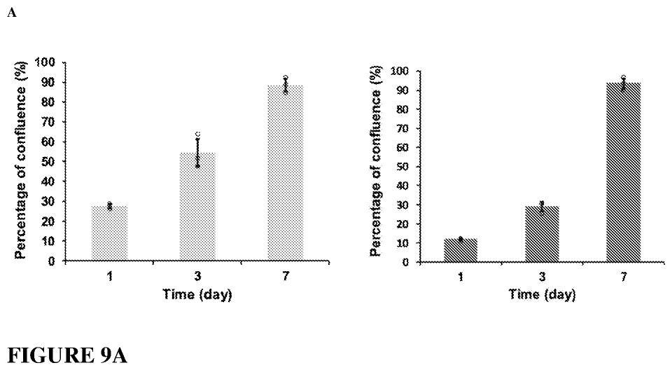

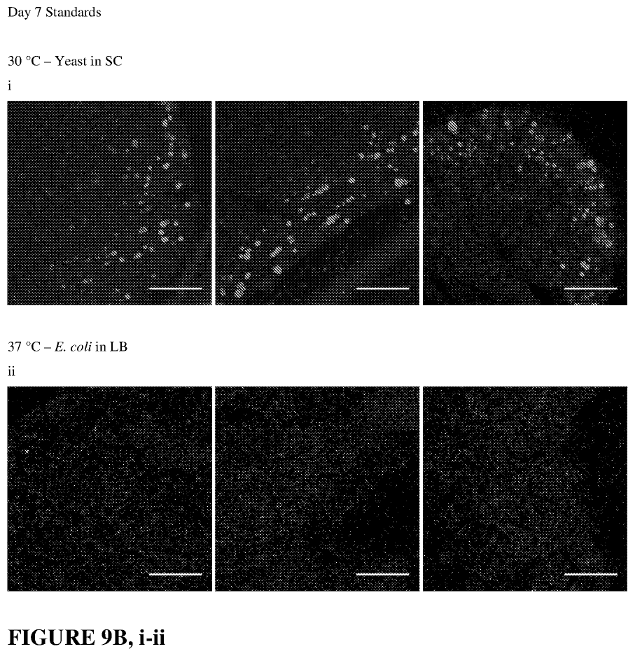

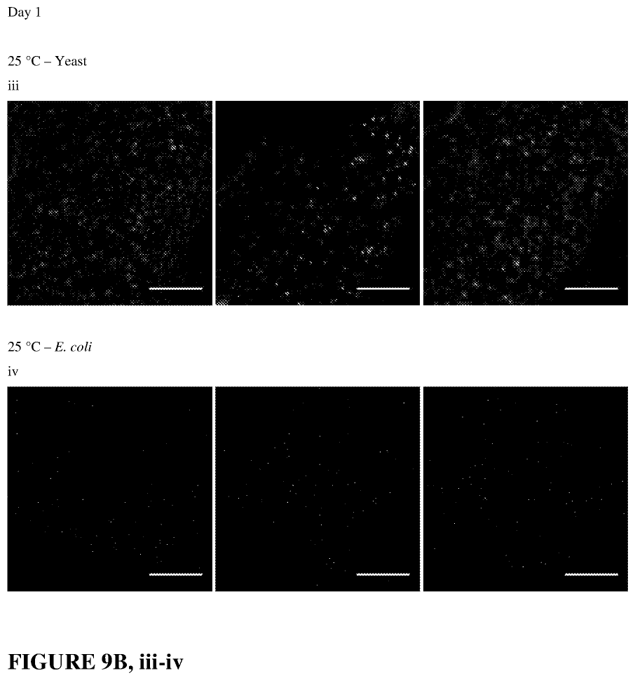

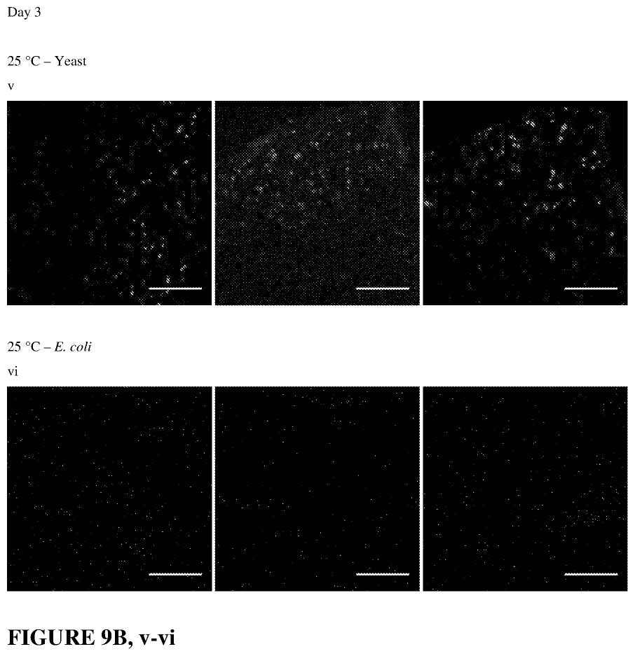

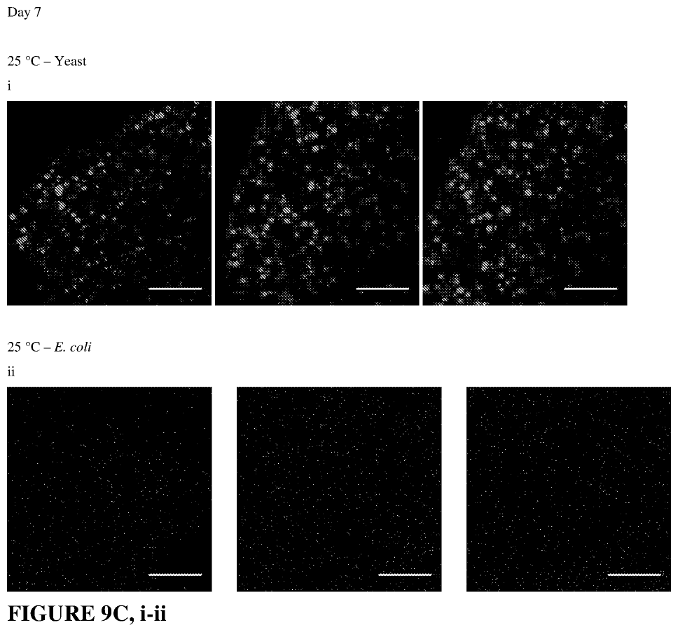

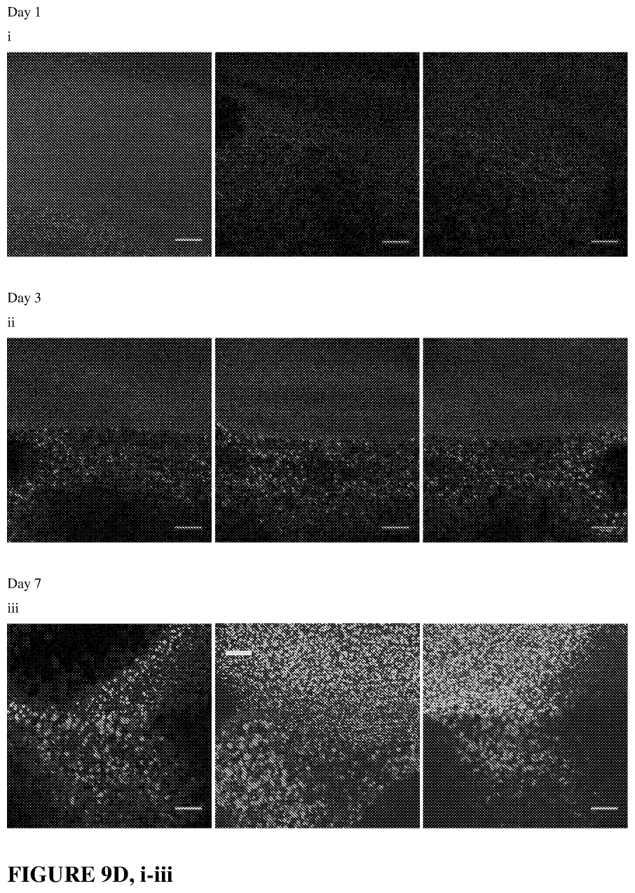

The experiments outlined to this point illustrate the use of mono-culture based microbe-laden hydrogels. However, spatially organizing a consortium within printed hydrogel constructs may provide a facile manner to control dynamics, especially for the endpoint balance within a co-culture. Natural microbial consortia rely upon spatial organization, as demonstrated by insect hindguts and biofilms (Agapakis 2012). In this regard, a synthetic microbial consortium that mimics these natural systems can provide both spatial organization as well as a protective environment for the microbes to thrive non-competitively. Achieving stable liquid co-cultures is challenging due to different competitive growth rates exhibited by many organisms, especially at different temperatures. This behavior is evident in a simple S. cerevisiae/E. coli co-culture expressing RFP and GFP, respectively, where one organism quickly dominates the other (FIG. 8). As an alternative, the printing of microbe-laden hydrogels into spatially localized regions of each organism can minimize or remove this competition. For example, when an alternating striped pattern of RFP-producing S. cerevisiae and GFP-producing E. coli laden hydrogels was printed, confocal imaging and fluorescent macroscopy showed that cell colony expansion was localized to the respective yeast and bacterial regions (FIG. 3A and FIG. 9). Moreover, these gels did not impede cell growth when co-cultured, as the yeast samples achieved a maximum confluence of 93.5% and the bacterial samples achieved a maximum confluence of 88.6% in the hydrogel-based consortium relative to similar mono-culture gels (FIG. 9A). Importantly, the physical segregation and hydrogel-based immobilization of the distinct microbial species offers advantages in controlling consortium population dynamics when compared to a mixed liquid culture system. 3D printing capacity allows the final consortium composition to be specified over a broad range by simply changing the amount of each respective gel printed into the final structure, thus enabling a plug-and-play approach to consortium bioprocessing. To test the efficacy of these hydrogel inks to spatially organize stable consortia, two different types of consortia were explored: (i) a cross-feeding consortium for betaxanthins production and (ii) a parallel consortium for more efficient glucose and xylose utilization in fermentation.

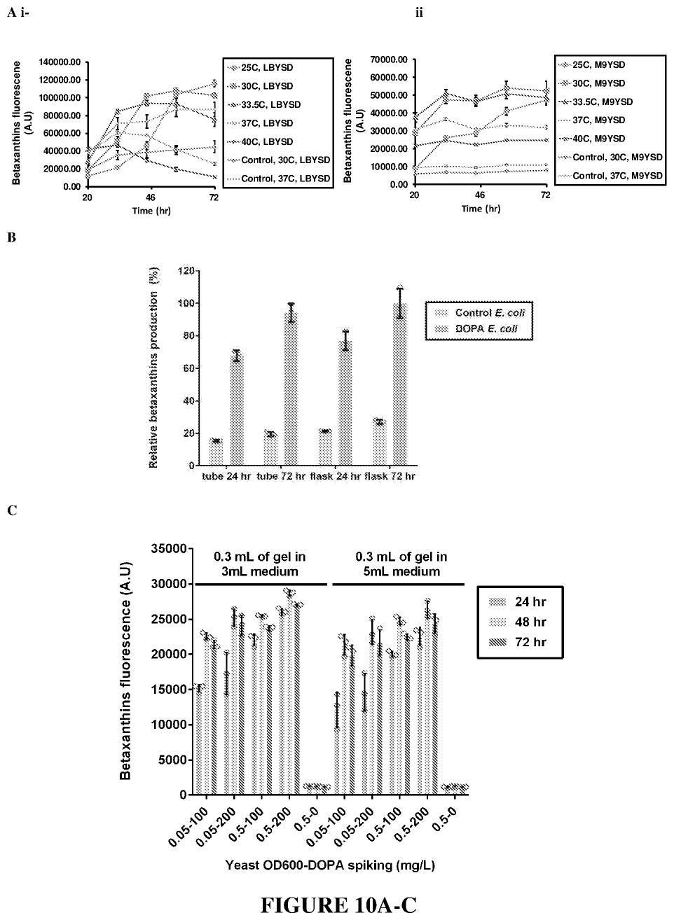

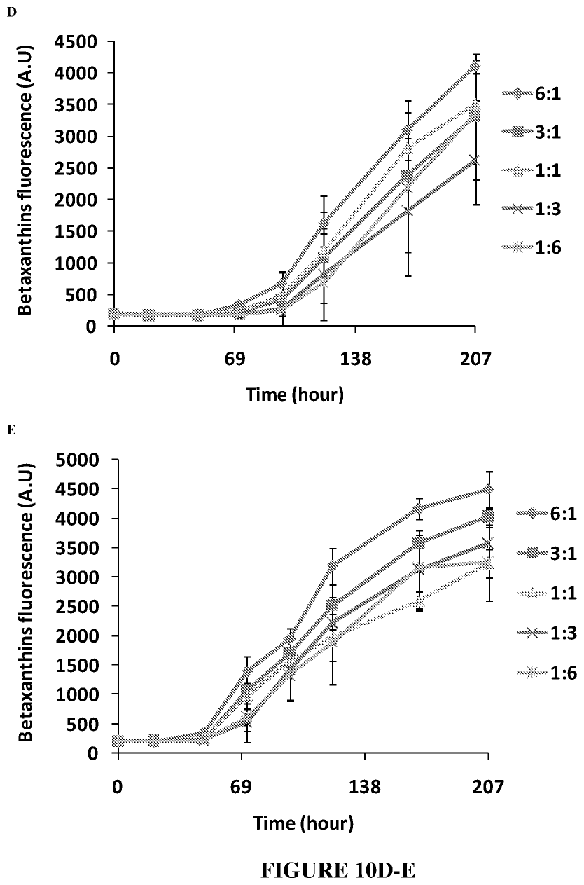

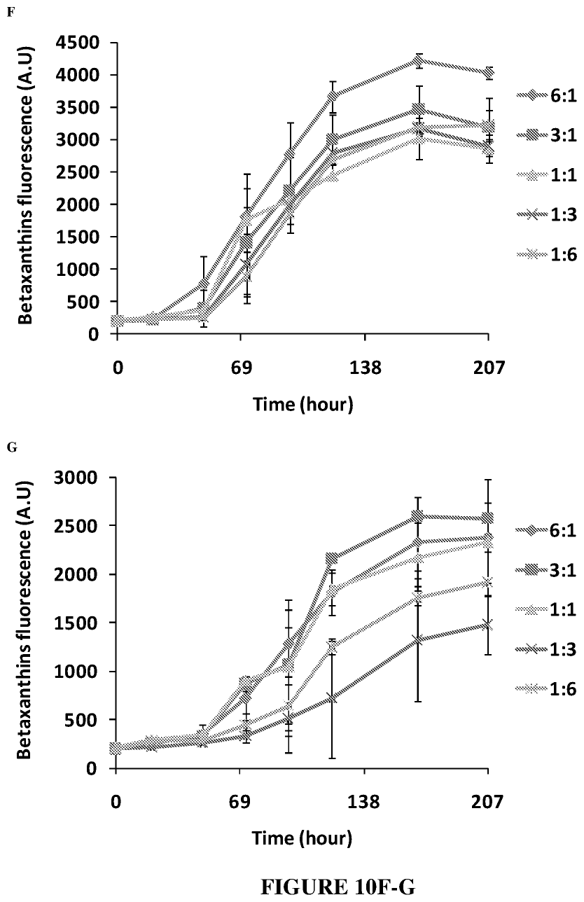

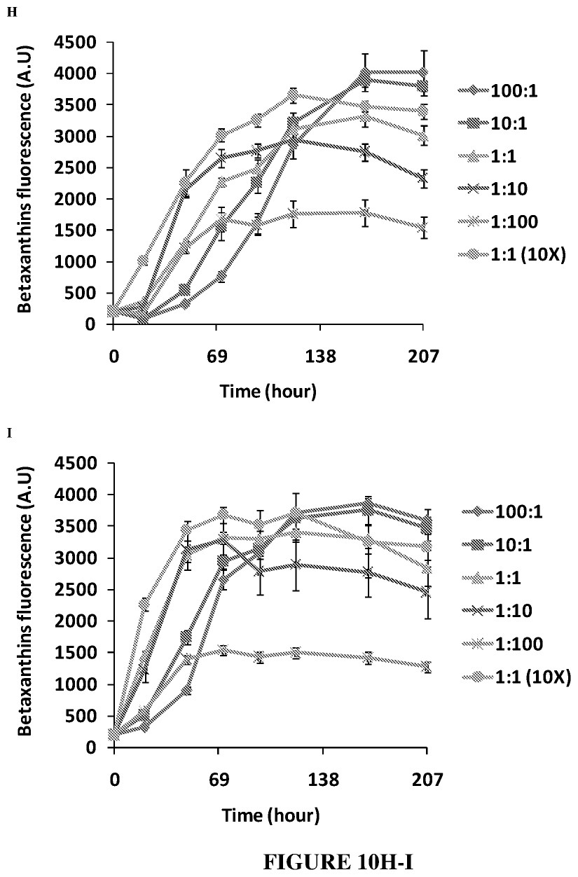

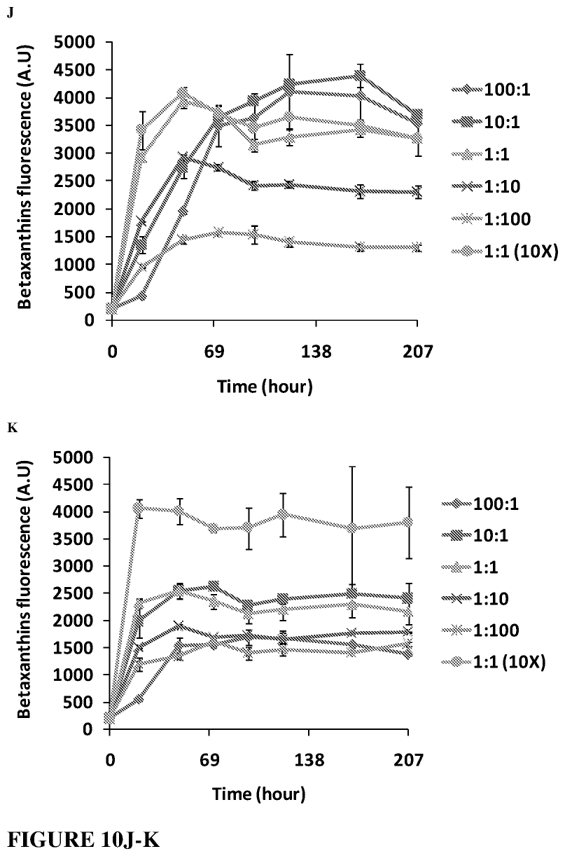

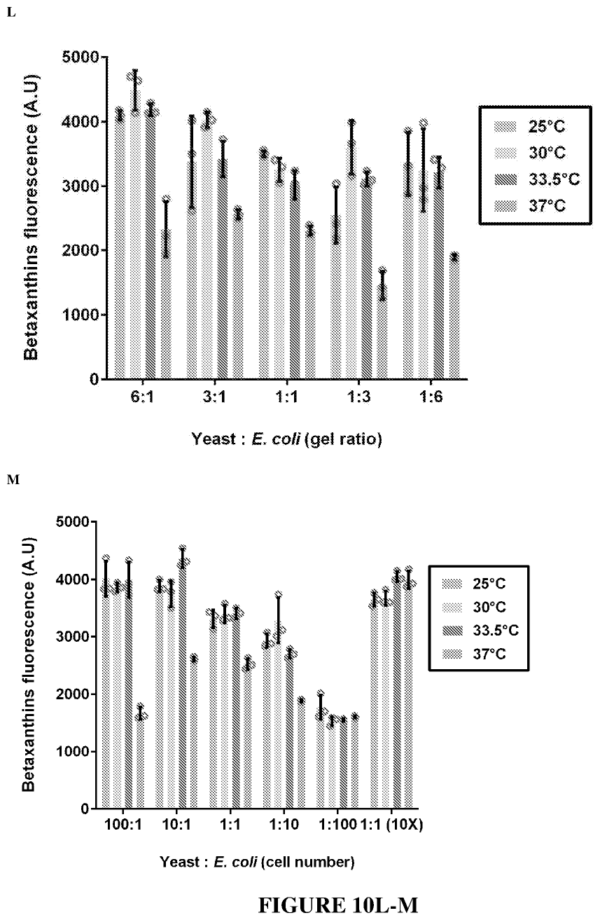

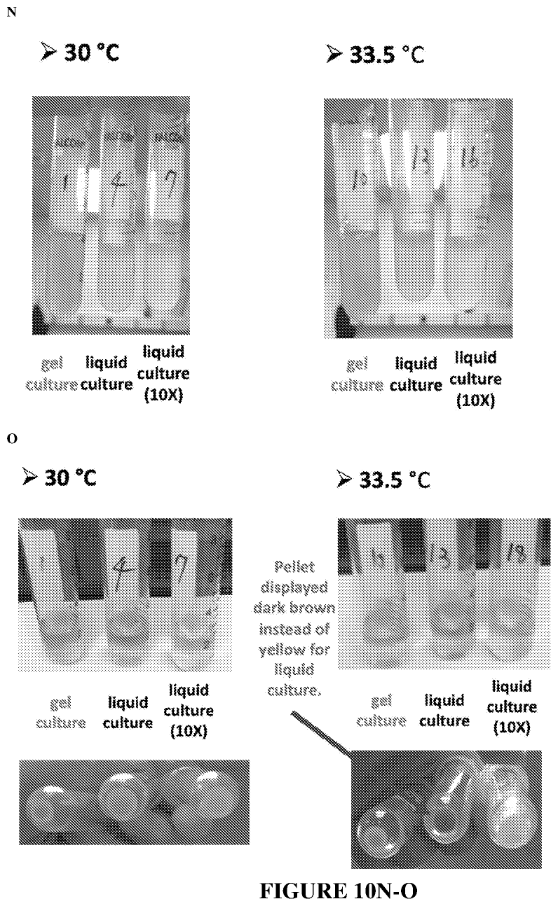

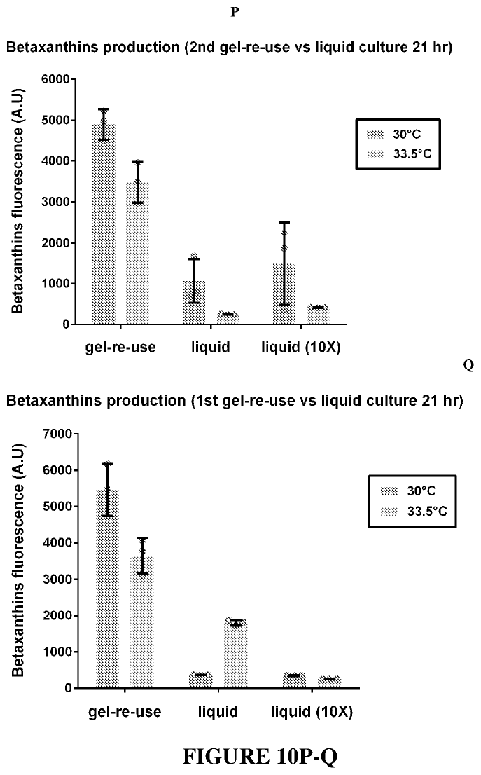

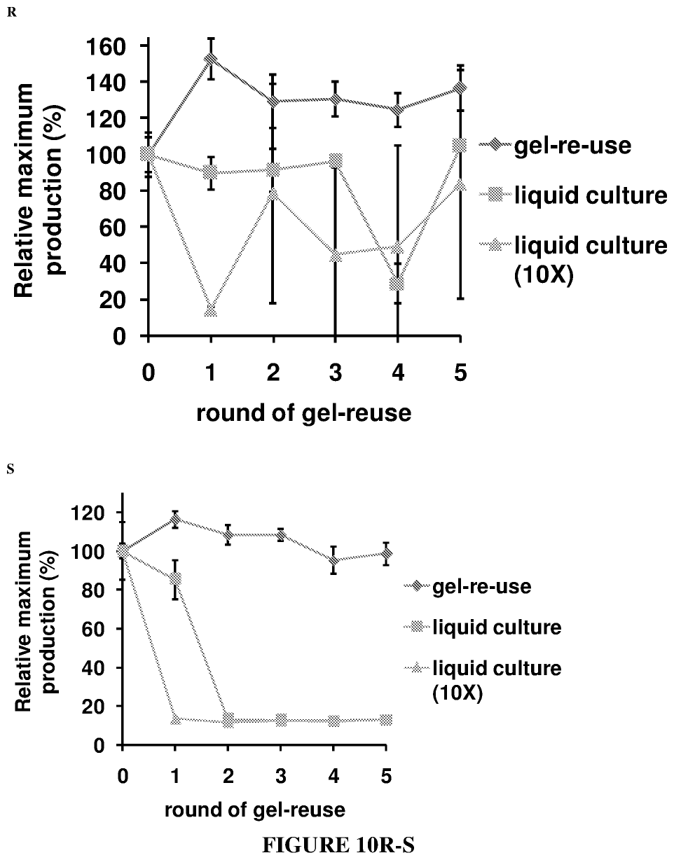

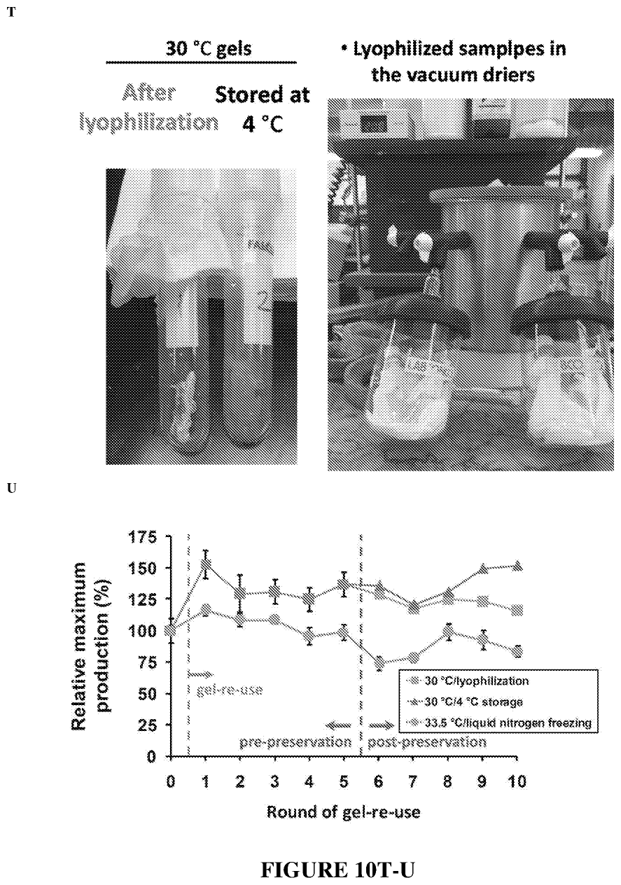

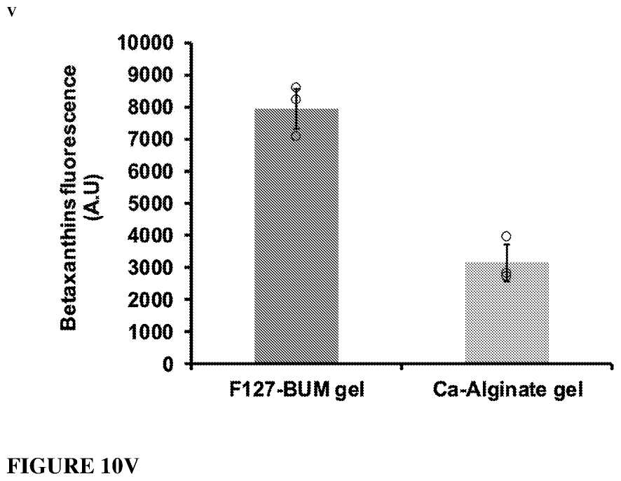

For a cross-feeding consortium, it was demonstrated the production of betaxanthins, a natural food colorant (Grewal 2018), through simple combination of an engineered E. coli and S. cerevisiae strain together to form a gel-based consortium (FIG. 3B). To do so, an L-DOPA producing bacteria-laden hydrogel was printed alongside a yeast-laden hydrogel that can convert L-DOPA into betaxanthins. No further engineering was conducted on these strains to enable mutualism or matched growth rates. To provide a qualitative comparison with liquid culture, the performance of this consortium was analyzed by assessing production of betaxanthins across a range of temperatures and cell ratios (enabled by altered inoculum ratios for the liquid culture and respective gel masses for the hydrogel system). Even in the round 0 condition, production by the hydrogel consortium surpassed the liquid suspension culture for most conditions and had a more consistent production response curve, especially across a wider range of temperatures (25.0-33.5° C.) (FIG. 3C and FIG. 10).

This consortium-based gel was also evaluated on the basis of reusability and on-demand production (FIG. 3D). Whereas the liquid culture quickly lost its ability to produce betaxanthins due to unstable co-culturing (FIG. 10), the metabolic activity of the immobilized consortium retained over 100% of the production compared to the round 0 hydrogel across five continuous re-use cycles at 33.5° C. (FIG. 10S). In fact, higher production was seen after round 0, possibly because the consortium was already mature and established, and thus the gels could more readily achieve a faster biocatalytic rate and economize sugar conversion (FIG. 10Q-S). These results for reusability prompted further investigation of preservation of microbial consortium-laden hydrogels for on-demand production of betaxanthins. In this case, lyophilization and re-use (similar to the scheme previously describe for mono-cultures) resulted in essentially the same production level over five subsequent fermentations (FIG. 3D), demonstrating that microbe-laden hydrogels containing co-cultures could function for a culturing period of at least 2 months. As with the ethanol mono-culture example above, all hydrogel samples were still operational at the end of this time period. To complement these preservation results, the impact of both refrigerated storage and liquid nitrogen freezing were studied on these consortium-containing gels (FIG. 3D and FIG. 10S). The refrigerated storage treatment exhibited no loss in productivity for an additional five rounds of re-use, whereas cryopreservation samples showed a mild reduction in metabolic activity, to 85% of the original productivity (FIG. 10U). In sum, the F127-BUM hydrogels used here provide a strong preservation capacity for consortia.

Next, a comparison of our F127-BUM hydrogel with standard calcium alginate encapsulation using the same consortium was explored. Calcium alginate hydrogels require constant replenishing of calcium ions in the reactor media in order to retain the charged crosslinks between the individual polymer strands. Additionally, the charged nature of the polymer itself may lead to unwanted interactions between the hydrogel and fermentation products, possibly interfering with diffusion out of the hydrogel (Cheetham 1979). In this comparison, a greater than 2.5-fold improvement in betaxanthins production for microbe-laden F127-BUM-based hydrogel was found when compared to microbe-laden calcium alginate (FIG. 10T). Moreover, it was observed that the microbe-laden calcium alginate gels softened and degraded slightly over a 72 hour incubation period, whereas the microbe-laden F127-BUM hydrogels retained structural integrity. These results demonstrate the re-use and stability of this platform compared with commonly used calcium alginate gels.

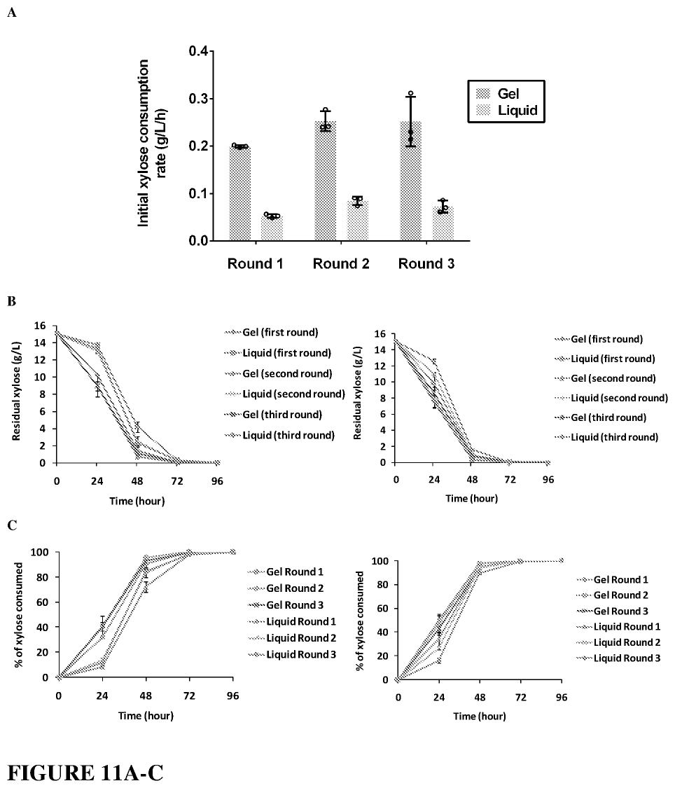

As a final demonstration, a parallel yeast-yeast strain consortium was assembled to enable glucose and xylose fermentation. In this case, a parallel consortium hydrogel comprised of a glucose-consuming wild-type S. cerevisiae S288C and a xylose-utilizing yeast YSX3 (Jin 2003) was compiled and net sugar utilization was compared to that of a free-cell, liquid culture consortia (FIG. 3E and FIG. 11). As in the betaxanthins example above, there is an implicit selection pressure in this system wherein the faster growing S288C wild-type strain will be enriched in glucose-rich conditions. The gel-based consortium mitigated this selection pressure and maintained a consistent xylose consumption rate of nearly 3.5-fold over the liquid culture condition when cells were initially cultured on glucose (FIG. 11A). Even when initial growth was conducted in a more xylose-strain permissive condition (such as YPDX in FIG. 3E), the gel-based consortium still outperforms the liquid culture format and remains stable, whereas the liquid consortium fails rather rapidly after only three subculture cycles. Collectively, these results highlight the stability and re-use advantages of spatially organizing consortium members within hydrogel constructs.

Through a series of proof-of-concept demonstrations, a 3D printing based platform has been developed that can spatially organize individual microbial populations and consortium members into hydrogel constructs for the production of both small molecules and active peptides. The approach enables repeated re-use and preservation through refrigeration or lyophilization, thus enabling on-demand production of these molecules in a manner that is unmatched by traditional liquid-based culturing. The ability to enable long-term stability of cells and consortia (for at least 1 year in the case of yeast fermentation of glucose to ethanol) provides a niche for preserving catalytic function in industrial bioprocesses. Moreover, the ability to control consortium dynamics simply by changing the amount of hydrogel ink printed provides a newfound capacity for plug-and-play synthetic consortia. Looking forward, this strategy enables a portable, reusable, and on demand capacity for small molecule and pharmaceutical production from a variety of microorganisms.

### Additional Information

Strains, Media and Plasmid Construction

All strains, plasmids and primers used herein are listed in Table 1 and Table 2. NEB10β was used for gene cloning or propagation of all expression vectors. It was cultivated in Luria-Bertani (LB) medium (10 g/L tryptone, 5 g/L yeast extract and 10 g/L NaCl) supplemented with appropriate antibiotics (100 μg/mL ampicillin, 50 μg/mL kanamycin or 50 μg/mL spectinomycin (Sigma)) with 225 rpm orbital shaking at 37° C. Starter cultures of yeast strains were routinely grown in yeast synthetic complete (YSC) media formulated using 6.7 g/L yeast nitrogen base (Difco) and 20 g/L glucose (MP Biomedicals) with the appropriate dropouts for auxotrophic selection. YPD (10 g/L yeast extract, 20 g/L peptone and 20 g/L glucose) and YPDX (10 g/L yeast extract, 20 g/L peptone, 15 g/L glucose and 15 g/L xylose) media were used in glucose/xylose utilization studies. LBYSD (1× LB, 1×YSC+CSM-URA, 10 mM vitamin C, 50 μg/mL kanamycin and 50 μg/mL spectinomycin) and M9YSD (1× M9, 2 mM MgSO₄, 0.1 mM CaCl₂), 1× YSC+CSM-URA, 10 mM vitamin C, 50 μg/mL kanamycin and 50 μg/mL spectinomycin) were used for medium optimization studies.

Oligonucleotide primers used for PCR amplification were purchased from Integrated DNA Technologies (Coralville, Iowa). For tyrosine production, Gibson assembly method was employed to combine an amplicon containing two T7 promoters amplified from pRSFDuet-1 (Addgene) with primers 11 as well as 12 and the PCR product amplified from pET28b empty vector (Addgene) with primers 13 and 14 to construct pET28b-Duet-1. To construct pCDF-pT7-tyrA^{(fbr)}-pT7-aroG^{(fbr)}), the FRT-flanked kanamycin resistance gene on the plasmid pCDF-Kan^{FRT}-tyrA^{(fbr)}-aroG^{(fbr)} (Wu et al. PLOS ONE 9, e101492 (2014)) was removed using primers P7-P10 via Gibson assembly. To construct pCDFDuet-1, two T7 promoters amplified from pRSFDuet-1 with primers 11 and 12 was Gibson assembled with the PCR product amplified from pCDF-pT7-tyrA^{(fbr)}-pT7-aroG^{(fbr)} with primers 13 and 14. To construct pET28-pT7-aroG^{(fbr)}-tT7, the DNA fragment PCR-amplified from pCDF-pT7-tyrA^{(fbr)}-pT7-aroG^{(fbr)} with primers P25 and P26 was cloned into the amplicon amplified from pET28b empty vector with primers P27 and P28. For pET28-pYIBN-aroG^{(Tr)} construction, native promoter yibN and DNA fragment containing aroG^{(fbr)} gene was PCR-amplified from E. coli MG1655 genomic DNA with primers P33 as well as P34 and from pET28-pT7-aroG^{(fbr)}-tT7 with primers P35 as well as 36, respectively. The resulting two amplicons were then cloned into the PCR product amplified from pET28b-Duet-1 with primers P37 and 38 using Gibson assembly. To construct pET28-pYIBN-aroG^{(fbr)}-B30rbs-tyrA^{(fbr)}-tRRNC, DNA fragment containing tyrA^{(fbr)} PCR-amplified from pCDF-pT7-tyrA^{(fbr)}-pT7-aroG^{(fbr)} with primers P41 as well as P42 was combined with primer 43 and cloned into the amplicon obtained from pET28-pYIBN-aroG^{(fbr)} with primers P39 as well as P40. For L-DOPA production, primer set P69 and P70 were used for amplifying HpaB-HpaC from BL21 (DE3) genomic DNA, and the resulting amplicon as well as primer 67 were Gibson assembled with the PCR product amplified by using primers P72 and P73 from the template pCDFDuet-1 to construct pCDF-pLPP-B30rbs-hpaB-hpaC-T7t. For the construction of pCDF-pLPP-B32rbs-hpaB-hpaC-T7t, primer set P70 and P71 were used for amplifying HpaB-HpaC from BL21 (DE3) genomic DNA, and the resulting amplicon as well as primer 68 were Gibson assembled with the PCR product amplified by using primers P72 and P73 from the empty vector pCDFDuet-1. For betaxanthins production, primer set P74 and P75 were first used for amplifying MjDOD gene from pCMC0759. The resulting amplicon was then linearized with restriction enzymes SpeI as well EcoRI and ligated with SpeI/EcoRI digested mumberg plasmid p416-pGPD-tPRM9 to yield p416-pGPD-MjDOD-tPRM9. To generate an URA3 integrative cassette, primers P78 and P79 were used for amplifying a linear DNA fragment with 65 bp-long Leu2 homology arms from p416-pGPD-MjDOD-tPRM9.

2,3-Butanediol Production in Yeast with Lyophilization and Re-Use

2,3-Butanediol is a building block biochemical for a variety of downstream chemicals including methyl ethyl ketone, acetoin and 1,3-butadiene with applications in synthesizing plastics, food additives or industrial solvents (Ji et al. Biotechnol. Adv. 29, 351-364 (2011)) 2,3-BDO has a global market demand of around 32 million tons per year with a value of $43 billion (K6pke 2011). As a result, microbial 2,3-butanediol production has been extensively studied in the past few decades. Previously, it was demonstrated that a multiplexed dCas9-based regulation system to simultaneously knock down the NADH sinks including ADH1/3/5 and GPD1 along with overexpression of endogenous NADH-dependent BDH1 enzyme can increase the production of 2,3-BDO by nearly 2-fold in a 2,3-BDO-producing S. cerevisiae CEN.PK2-a strain (harboring a heterologous pathway with overproducing NoxE, alsD and alsS enzymes) (Deaner 2018) (FIG. 2a).

To evaluate the efficacy of the microbe-laden hydrogel system, two 2,3-BDO-producing yeast strains (BY4741 and CEN.PK2-a) were each encapsulated in the gel matrix and performance was compared pre- and post-preservation (FIG. 4). The 2,3-BDO productivity between the two engineered strains of BY4741 (0.035 g/L/h) and CEN.PK2-a (0.034 g/L/h) were comparable at 48 hours before lyophilization. After lyophilization, the 2,3-BDO productivity in preserved BY4741 gel (0.03 g/L/h) retained nearly 90% efficiency compared to the untreated sample, while the biological productivity in lyophilized CEN.PK2-a hydrogels decreased to 0.025 g/L/h (about 70% efficiency). Part of the decrease in efficiency seen with 2,3-BDO in the CEN.PK2-a strain was a result of an increased acetoin byproduct titer seen in this condition that increased from 1.1 g/L (pre-lyophilization) to 1.8 g/L (post-preservation) at the 48-hour timepoint (FIG. 4B). Acetoin is formed from the bioconversion of 2,3-BDO into acetoin via reversible yeast butanediol dehydrogenase (BDH1) enzymatic reactions (Gonzilez 2010). Regardless, the net metabolic activity (acetoin+2,3-BDO) evaluated for both pre- and post-lyophylization is indicative of sustained metabolic activity by the yeast-laden gels.

L-DOPA Production in E. coli

L-DOPA can be biosynthesized from tyrosine and is a precursor of the neurotransmitter dopamine in human (Broadley 2010) and has also been used as a drug for treating Parkinson's disease (Miguelez 2017). As a result, microbial production of L-DOPA (Surwase 2011; Munoz 2011) from low-cost lignocellulosic feedstocks is an attractive process compared to direct extraction from plants restricted by the low yield and purity issues (Yuan 2018; Yuan 2017).

Previous studies have shown that introduction of feedback-inhibition-resistant 3-deoxy-d-arabinoheptulosonate-7-phosphate (DAHP) synthase (encoded by aroG^{fbr}) and chorismate mutase/prephenate dehydrogenase (encoded by tyrA^{fbr}) in a tyrR (encodes a transcriptional regulator of aromatic amino-acid biosynthesis) knockout E. coli can sufficiently overproduce tyrosine. In order to redirect metabolic flow from the intracellular tyrosine pool to the L-DOPA biosynthesis pathway, 4-Hydroxyphenylacetate 3-hydroxylase (HpaBC) was overexpressed in the tyrosine-producing strain eBL04 (FIG. 2b). As expected, expression of hpaBC under the control of a constitutive promoter with a strong synthetic ribosomal binding site resulted in E. coli eBL0430D exhibiting a higher titer of L-DOPA (around 213 mg/L) than that of the eBL0432D strain that has a weaker overall expression (about 160 mg/L) (FIG. 5). Additionally, a change in coloration of culture broth for both engineered strains was observed, indicative of the oxidation of L-DOPA to form brown melanin-like pigments (Huang 2008).

Peptide Antibiotic Production in E. coli with Lyophilization and Re-Use

To expand beyond small molecules, it was demonstrated that microbe-laden hydrogels are capable of producing small peptides—in this case, biosynthesis of peptide antibiotic colicin V (colV). ColV is secreted by E. coli and other members of Enterobateriaceae and previous studies have identified that four genes cvaA, cvaB, cvaC and cvi of the colicin V gene cluster are required for ColV synthesis, export, and native immunity (Gilson 2007; Gérard 2005) (FIG. 7a). The colicin V-producing strain expressing its cognate immunity protein Cvi alone is sufficient to protect itself from the killing activity of ColV. The extracellular secretion of ColV, encoded by the cvaC gene, is mediated by an efflux system containing cytoplasmic proteins CvaA and CvaB as well as the host outer membrane protein TolC, then the CvaA-CvaB-TolC exporter recognizes the N-terminal 15-amino-acid leader sequence of primary translation polypeptide ColV and proteolytically processes it to render a mature 88-amino-acid ColV peptide. The secreted ColV is only active against sensitive cells when inserted into the inner membrane of a target cell resulting in membrane depolarization.

In this example, ColV antibiotic-producing E. coli that was expressing cvaA, cvaB, cvaC and cvi genes was encapsulated in the F127-BUM hydrogel matrix. Using the printed gels, two bactericidal assays were conducted using the broth supernatant of the culture. These assays included a zone of inhibition test and bacterial suspension broth test to evaluate the antimicrobial activity of ColV. As shown in FIG. 2d, the inhibition halo yielded by the gel system was consistently observed over four consecutive repeated uses after lyophilization, indicating the cell functionality in the gels was still retained and the diffusion of the peptide from the gel matrix was not inhibited. The bactericidal activity was not affected by the preservation process for the first two repetitive uses and the efficiency was reduced to only around 80% for the last batch of re-use compared to the original (round 0). The average antimicrobial activity for four subsequent rounds of re-use after lyophilization was nearly 100% of the original gel based on the measurement of diameter of clear zones appeared on agar plates. In addition, the production of the secretory peptide antibiotic in gels was observed in the broth test (FIG. 7c) despite the slight reduction in antimicrobial functionality based on the inhibition zone test for the last two rounds of re-use (FIG. 7b). Thus, the overall production was maintained across the rounds of re-use using a mixture of bacteriastatic and bactericidal assays.

For benchmarking, the bactericidal activity of the liquid culture system was also compared to that in the hydrogel system (FIGS. 7b and 7c). The zone of inhibition test (FIG. 7b) and broth test (FIG. 7c) clearly demonstrated that the antimicrobial peptide activity made in a free-cell system was inconsistent for iterative cell-re-use compared to the hydrogel system. In the 3^{rd} re-culturing round, the inhibition zone in free-cell system was actually not observed and its bactericidal activity dramatically was reduced to 6.5%. Likewise, the larger error bars on the liquid culture demonstrates the extreme variability of samples from biological replicates indicative of the instability experienced in repeated liquid subculturings.

Overall, these results showcase the capability of the described hydrogel system in maintaining high cell density and supporting protection to lyophilization, thus resulting in a continuous operation of peptide antibiotic production. Moreover, it is expected that the printed gel can carry a higher next cell density/concentration than a microbial suspension system (Eş 2015; Alonso 2016), thus leading to a stronger and more consistent production level.

Betaxanthins Production Via a Synthetic Cross-Feeding Consortium

Betaxanthins are water-soluble natural yellow pigments that have been proposed as a substitute for artificial yellow dyes (Martins 2017). Previous studies have established a de novo plant betaxanthins biosynthesis pathway in S. cerevisiae for high-throughput screening of tyrosine hydroxylase mutant library and for the investigation of spectral and physical properties of various amine-betaxanthins (Grewal 2018; DeLoache 2015). A synthetic E. coli-yeast consortium was created (FIG. 3b) whereby the L-DOPA secreted by engineered E. coli is consumed by the DOPA-4,5-dioxygenase (DOD)-overproducing yeast that converts L-DOPA into betalamic acid. The free betalamic acids then condense with primary or secondary amines to yield the fluorescent yellow betaxanthin pigments.

To evaluate the impact of medium components on fermentative performance, free cell, suspension cultures were evaluated using E. coli eBL0430D-S. cerevisiae sBY08 were tested at different temperatures (FIG. 10a). Betaxanthins titers gradually increased over time at 25° C. for both LBYSD and M9YSD conditions. Although the maximum titer of betaxanthins for each condition was nearly 2-fold higher when using LBYSD medium compared with using M9YSD, the production significantly declined over time for higher temperatures 33.5 to 40° C., suggesting using M9YSD medium could provide a more stable condition for the product. Thus, M9YSD medium was chosen for the following studies. A consistent correspondence of betaxanthins titer between fermentation conducted in flask scale and tube scale was found (FIG. 10b); therefore, experiments were conducted at the tube scale for operational convenience and condition multiplexing. Previous studies have demonstrated DOD reaction requires oxygen (Grewal 2018). To verify the effect of aeration for test tube culture on betaxanthins production in the gel system, the DOD-yeast-laden hydrogel was printed and cultured in a working volume of 3 mL or 5 mL medium supplemented with exogenous L-DOPA (FIG. 10c). Increasing the concentration of spiked L-DOPA or encapsulated cell biomass in gels enhanced betaxanthins production. Fermentation performed with 3 mL working volume led to a 1.2-fold increase in production compared to that with 5 mL medium, potentially due to the increased oxygenation; therefore, a 3 mL working volume was adopted for the subsequent experiments.

To further explore the impact of fermentation temperature and initial cells density on betaxanthins production, varying temperatures and cell ratios were evaluated for both liquid culture and gel systems (FIG. 3c and FIG. 10d-1). To control gel ratios, various amount of gel was printed and cured. To control liquid culture ratios, different amounts of starting culture was re-suspended. Each printed gel carried the same initial yeast or E. coli cell concentration of 1.5×10⁸ cells per gram of polymer. In liquid culture, the initial cell ratio was altered, ranging from 100:1 to 1:100 while keeping the initial net yeast and E. coli cell concentration constant at 3×10⁶ cells per mL of culture. As shown in FIG. 3c and FIGS. 10d-l, the production using gel system surpassed the microbial suspension culture system for most conditions. The consortia productivity in gel system with a gel ratio of 1:6 outcompeted the liquid culture with cell number ratio 1:100 by nearly 2-fold, especially across a range of temperatures (25-33.5° C.). For 1:1 ratio at 25° C., the gel system produced more betaxanthins compared to liquid system. At this condition, despite the starting point containing equal amount of E. coli and yeast cells, the consortia composition in suspension culture could be biased towards having more yeast than bacteria since 25° C. is closest to the yeast optimal growth temperature. In contrast, the gel based system can better control the end-dynamics of the consortium which is important for improving production.

Next, the reusability of the consortium-laden gels was explored (FIG. 3d and FIG. 10m-q). The activity throughout five consecutive rounds of re-use was determined by comparing the performance of gels with 6:1 ratio (as measured by the comparing the highest betaxanthins titer measured at 30 and 33.5° C. to that of the round 0 experiment) (FIG. 101; top) to that using the liquids with 100:1 and 1:1 (10×) (the condition mimicking the same amount of initial cell number used in the 1:1 gel ratio condition) ratios (FIG. 101; bottom). The gels retained their active metabolism and continuously produced betaxanthins and the titer substantially out-competed that of the free-cell fermentation system for both the 30 and 33.5° C. conditions (FIG. 10o-q). Importantly, the metabolic activity of the immobilized consortium was retained and exceeded 100% activity when compared with the original gel (round 0) for five continuous re-use at 30° C. (FIG. 10p). The consortia activity of gel at 33.5° C. was 100% and 95% of the original activity for the first three rounds and last two rounds of re-use, respectively (FIG. 10q). Although betaxanthins production for the round 0 took longer than that of a free-cell system (FIG. 10d-g), once the steady state of both embedded species was established, the gels rapidly produced their maximum biocatalytic rate for the next re-use rounds (FIG. 10m-o).

In contrast, the free-cell system at 30° C. performed a in a fluctuating manner and did not yield consistent betaxanthins titer for the five consecutive re-culturings (FIG. 10p). In fact, two of the biological triplicates could not make the betaxanthins after subculturing, suggesting that liquid culture system is unstable and subject to random consortia dynamics. Additionally, the free-cell system at 33.5° C. rapidly lost all ability to make betaxanthins (FIG. 10q). Moreover, the cell pellets of the 100:1 liquid samples at 96 hour fermentation timepoint at 33.5° C. for the first round of cell-re-use displayed brown pigments (FIG. 10n), likely due to the overabundance of E. coli and the formation of melanin-like by-product yielded from oxidation of L-DOPA. In addition, the pelleted cells showed no color change for 1:1 (10×) samples at the same condition. These results suggest the end consortia composition for continuous cell use experiment was varying through liquid culture. It should be noted that the titer obtained from the gel system may also be underestimated, due to some yellow pigmentation observed as being retained in the gel after fermentation.

Maintaining cell viability is paramount to industrial-scale bioprocesses. Three different treatments for preservation of this consortia were conducted including lyophilization, refrigerated storage and liquid nitrogen freezing (FIG. 3d and FIGS. 10r and 10s). The lyophilization process resulted in no loss of consortia productivity over five subsequent repeated fermentations after the fifth round of initial re-use (FIG. 3d). The biocatalytic efficiency at 30° C. was still retained at nearly 120% compared to the round 0 experiment. In addition, the gel after refrigerated storage treatment for one week still exhibited average consortia productivity at around 140% of the original for the last five rounds of re-use carried out at 30° C. (FIG. 3d). The control of cooling rate as well as the use of cryoprotectants have been demonstrated to improve cell viability and long-term stability. However, it is shown herein that this hydrogel ink is capable of providing protection from these conditions. To further demonstrate this, liquid nitrogen was used to freeze the gels that were selected from the round 5 experiment at 33.5° C., and proceeded to store the frozen gels at −80° C. overnight. The consecutive re-use study even on these gels showed that the cell viability was not severely affected by these freezing conditions, a condition that usually causes mechanical damage of cell membranes due to the formation of large ice crystals or irreversible protein denaturation. Specifically, the average metabolic activity of entrapped consortia reduced to only nearly 85% of original for the last five repeated batches (FIG. 10s).

Xylose/Glucose Utilization Via a Yeast-Yeast Consortium with Repeated Gel-Re-Use Xylose is the most abundant components in hemicellulose of lignocellulosic feedstock (Gírio2010), however, conventional wild-type yeast S. cerevisiae is not capable of assimilating xylose (Kwak 2016). Previous studies have demonstrated a number of methods to import this metabolism including heterologous overexpression of Scheffersomyces stipitis xylose reductase (XYL1), xylitol dehydrogenase (XYL2) and D-xylulokinase (XYL3) in S. cerevisiae resulting in a xylose-utilizing strain YSX3 improved cell growth and ethanol production from xylose (Jin 2007).

The hydrogel system disclosed herein was used to enable a yeast-yeast parallel consortium. Wild-type S. cerevisiae S288C along with an engineered xylose-utilizing yeast, YSX3, were each individually encapsulated in hydrogels and the glucose/xylose consumption efficiency was compared with the liquid culture system (FIG. 3e and FIG. 11). The sugars utilization rate was not determined for the round 0 since the purpose of this round was to outgrow and establish the consortia population. Two separate experiments were performed in parallel for the round 0: one with the yeast-yeast co-culture in YPD medium containing only 20 g/L of glucose (FIG. 11a), and the other in YPDX medium comprising 15 g/L of xylose and 15 g/L of glucose (FIG. 3e). In all cases of the re-use (for both gels and suspension systems), glucose was fully used within 24 hours and the systems exhibited a diauxic shift. As shown in FIG. 11a, the gels initially maintained in YPD medium for round 0 displayed nearly 3.5-fold improvement on the average of initial xylose consumption rate over the liquid cultures during three subsequent rounds of re-use. Additionally, a lag phase in xylose consumption rate for the first round of re-use in both systems was observed (FIG. 11c; left), due to the shift in metabolism. Furthermore, the free-cell system at this condition exhibited lower average xylose consumption rate (around 0.07 g/L/h) (FIG. 11c; left) than that in the liquid culture conducted in YPDX for round 0 (about 0.16 g/L/h) (FIG. 11c; right), suggesting the suspension yeast cultures grown in YPD after round 0 probably were already self-selected to possess a higher proportion of wild-type yeast (as this is the faster, more robustly growing strain). Additionally, the performance of liquid culture progressively declined over each round of the re-use (FIG. 3e) and the xylose consumption rate for the round 3 (around 0.1 g/L/h) approached that seen in FIG. 11a (about 0.07 g/L/h), indicating the free-cell system could not properly maintain the dynamics of the consortium. In contrast, the consortia-laden hydrogels exhibited a higher xylose consumption rate (0.3 g/L/h on average) compared to the liquid culture system over each round of repetitive use with the increasing improvement on xylose utilization (1.5-fold increase in round 1, 1.8-fold increase in round 2, and 2.7-fold improvement in round 3) (FIG. 3e).

### Example 2: Protein Production by Mammalian Cell Cultures in Cell-Laden Hydrogels

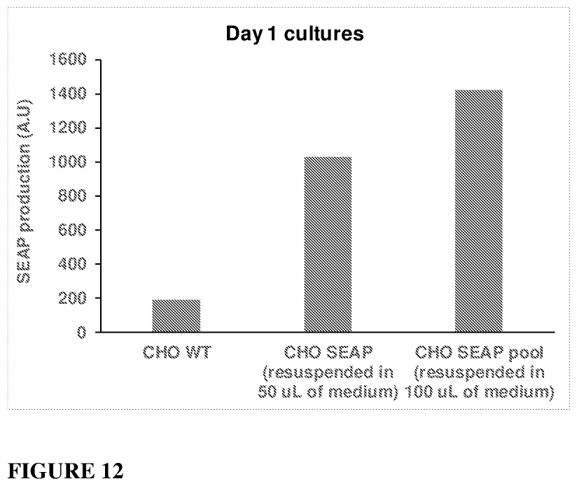

To demonstrate the compatibility of the hydrogel ink with mammalian cells, a stable-pool of Chinese Hamster Ovary Cells (CHO-DG44) transfected with a construct to produce SEAP as a model secreted protein was encapsulated. In similar fashion as the previous examples, these cells were cross-linked into the gel and printed. After incubation of the gel in culture media, SEAP levels were detected in the supernatant (FIG. 12). These results demonstrate that mammalian production hosts (such as CHO) are compatible in this gel system and the gel enables the diffusion of the produced protein.

### Materials and Methods

### Plasmid Construction

To construct secreted placental alkaline phosphatase (SEAP) producing plasmid TB-pEF1a(E2)-SIZ-SV40 pA, human EF1a constitutive promoter was amplified from the plasmid TB-pEF1a(E2)-hrGIZ-SV40 pA (EcoRV) using forward primer GAGTAATTCATACAAAAGGACTCG (SEQ ID NO: 41) and reverse primer TTTGGCTTTTAGGGGTAGTTTTC (SEQ ID NO: 42). The resulting DNA fragment was Gibson assembled with the amplicon obtained from TB-SIZ-SV40 pA, which includes AscI restriction sites, with forward primer GTCGTGAAAACTACCCCTAAAAGCCAAAGCTAGCATGCTGCTGCTG (SEQ ID NO: 43) and reverse primer GGGCGAGTCCTTTTGTATGAATTACTCGCGGCCGCTTTTTTCCTTC (SEQ ID NO: 44). The plasmid was transformed and propagated in Escherichia coli NEB10β.

### Cell Culture, Growth and Transfection

The suspension-adapted wild-type CHO-DG44 cells were grown in a chemically defined CD DG44 medium supplemented with 8 mM glutamine and 0.2% pluronic F-68. Cell cultures grown in shake flasks were maintained at 37° C., 5% CO2, humidity 85% and 125 rpm.

For transfections, 10⁷ viable CHO-DG44 cells were transfected with 17.5 μg of AscI-digested linear DNA using Lipofectamine™ 2000 (Invitrogen) as described by the manufacturer. Surviving cells were then transferred to the growth medium and allowed to recover for 72 h prior to addition of Zeocin (Invitrogen) selection pressure. To establish a stable pool, the transfected cells were then periodically transferred into medium containing 200 μg/mL Zeocin until the viability reaches to 98%.

To measure SEAP production, the supernatant obtained from centrifugation to pellet cells was sampled for analysis using the NovaBright Secreted Placental Alkaline Phosphatase (SEAP) Enzyme Reporter Gene Chemiluminescent Detection System 2.0 (Invitrogen). Luminescence was Measured with a BioTek Citation 3 at Emission Detection range from 400 nm to 650 nm resulting in maximal emission at of 540 nm.

### SEAP Production in Hydrogel

As a negative control, 2×10⁶ viable cells of wild-type CHO-DG44 were suspended in 100 μL of growth medium and embedded in 0.3 gram of polymer. 2×10⁶ viable cells of SEAP-producing CHO-DG44 were individually suspended in 50 or 100 μL of growth medium and encapsulated in 0.3 gram of polymer. Wild-type cells were grown in the 2.5 mL of medium without antibiotic, and SEAP-producing cells were cultured in the growth medium supplemented with 200 μg/mL Zeocin using a 6-well shaking plate at 37° C., 5% CO₂, humidity 85% and 125 rpm. After culturing for 24 hours, the SEAP level in supernatant of each sample was analyzed using the SEAP reporter assay as described above.

### Example 3: Bioproduced Proteins on Demand (Bio-POD) in Hydrogels Using Pichia pastoris

Traditional production of industrial and therapeutic proteins by eukaryotic cells is well utilized in industry, but typically requires large-scale fermentation capacity. As a result, these systems are not easily portable or reusable for on-demand protein production applications. Disclosed herein is Bioproduced Proteins On Demand (Bio-POD), a hydrogel-based technique to immobilize engineered Pichia pastoris for production and secretion of medium- and high-molecular weight proteins (in this case, SEAP, α-amylase, and Trastuzumab). The encapsulated-yeast gel samples exhibited minimal loss of productivity after lyophilization and re-use and outperformed a traditional suspension system. The hydrogel platform described here is the first hydrogel immobilization employed successfully to produce recombinant proteins using a P. pastoris system. These results highlight the potential to establish a cost-effective bioprocessing strategy for on-demand protein production.

In Example 1, a temperature-responsive, shear-thinning F127-BUM hydrogel for on-demand biomolecule production using either mono- or co-cultures was demonstrated to provide preservation capacity (i.e. metabolic activity of lyophilized consortia hydrogels retained 100% efficiency after long-term storage at room temperature for 3 months) and reusability (i.e. yeast-laden hydrogels enabled ethanol production for over 1 year of repeated use). It has also been demonstrated that this approach is suitable for small molecules and very small polypeptides (as with the case for colicin V production). Importantly, this hydrogel system was quite compatible with yeast hosts.

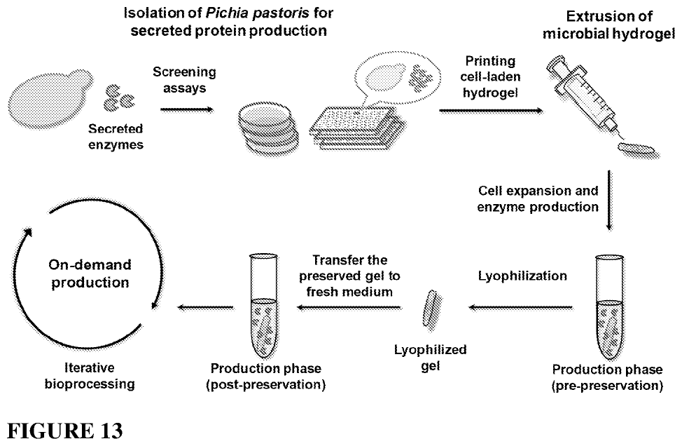

In this example, it is demonstrated that engineered P. pastoris can be embedded within this same F127-BUM hydrogel-ink to enable preservable and reusable production of secreted recombinant proteins of increasing size (FIG. 13). Through this approach, Bioproduced Proteins On Demand (Bio-POD) were enabled. Specifically, it was shown that secreted proteins are able to effectively diffuse from within the printed yeast gel into the surrounding culture media, thus allowing for facile product harvest and for hydrogel re-use. Additionally, it is possible to preserve these hydrogel inks through lyophilization and retain productivity that is on par with that of pre-lyophilized samples in a manner that outcompetes liquid culture with respect to consistency over multiple rounds of sample reuse.

Materials & Methods

Strains, Media and Plasmid Construction

All strains, plasmids, primers and a gBlock gene fragment used in this study are listed in Tables 3-5. Oligonucleotide primers used for PCR amplification were purchased from Integrated DNA Technologies (Coralville, Iowa). All Gibson-assembled DNA (Gibson 2009) were electroporated (2 mm Electroporation Cuvettes, Bioexpress) into E. coli competent cells with a BioRad Genepulser Xcell at 2.5 kV. E. coli NEB110β was used for gene cloning or propagation of all expression vectors. For propagation of pPIC9 series, NEB110β was cultivated in LB medium (1% tryptone, 0.5% yeast Extract and 1% NaCl) supplemented with 100 μg mL-1 ampicillin (Sigma). For propagation of pPICZα series, NEB110β was grown in low salt LB medium (1% tryptone, 0.5% yeast Extract and 0.5% NaCl, pH7.5) supplemented with 25 μg mL-1 zeocin (Sigma)) with 225 rpm orbital shaking at 37° C. Starter cultures of yeast strains were routinely grown in YPD (1% yeast extract, 2% peptone and 2% glucose) medium at 30° C. Electroporation of Pichia with an integrative expression cassette was performed according to Pichia Expression Kit's (Invitrogen) instructions. Minimal dextrose (MD) medium (1.34% YNB, 4×10⁻⁵% Biotin and 2% glucose) was used in auxotrophic selection study. Buffered glycerol complex medium (BMGY) and buffered methanol complex medium (BMMY) were prepared according to the manual of EasySelect™ Pichia Expression Kit (Invitrogen) and used in recombinant protein production.

For SEAP production, an amplicon containing a human SEAP gene amplified from the plasmid TB-SIZ-SV40 pA (Cheng 2016) using primers P1 as well as P2 was generated to replace the N-terminal human secretion signal peptide with S. cerevisiae α-factor mating signal sequence, and remove hydrophobic C-terminal signal peptide for PI-G anchor attachment (Heimo 1998). The amplicon was then Gibson assembled with the PCR product amplified from pPIC9 empty vector (Invitrogen) with primers P3 and P4 to construct pPIC9-SEAP. To construct pPICZα empty vector, the gene fragment amplified from the plasmid pPICZalphaB-SapL3 (Addgene) with primer P5 and P6 was Gibson assembled with primer P7. To construct pPICZα_AmyL, the amplicon without SapL3 gene was amplified from pPICZalphaB-SapL3 using primers P5 as well as P6, and Gibson assembled with the PCR product amplified from the amylase gBlock codon-optimized for P. pastoris (Table 5) with primer P8 and P9.

F127-BUM Hydrogel Preparation

Polymer functionalization, extrusion of yeast hydrogels, and photocuring procedures were performed by following the work shown in Examples 1 and 2. In short, 30 wt % F127-BUM polymer solution was mixed with photo-initiator (2.5 μL per Ig solution) to facilitate polymerization of the methacrylate functional groups upon UV exposure. Photo-initiated polymer solution was mixed with 4.5×10⁷ cells before extrusion to yield robust, viable microbe-laden gels upon brief UV curing.

SEAP Secretion and Production

The plasmids pPIC9 and pPIC9-SEAP harboring S. cerevisiae α-factor mating signal sequence were first digested by restriction enzyme SalI to promote insertion in the his4 locus. Then the linearized integration cassettes were separately transformed into P. pastoris GS115 by electroporation. MD agar plates and broth media were used for selection of His+ transformants. Five colonies from MD plates were picked and examined for production levels. Seeding cultures were grown in BMGY medium. Protein induction was performed using 15 mL of BMMY medium (0.5% methanol) with the initial OD₆₀₀ of 0.5 in a 125 mL flask. Cultures were incubated at 30° C. with an orbital speed 225 rpm. A final concentration of 0.5% methanol was aseptically added to the flask every 24 h to maintain protein induction. To measure SEAP production, the supernatants obtained from centrifugation to pellet yeast cells were taken for analysis using the NovaBright™ Secreted Placental Alkaline Phosphatase (SEAP) Enzyme Reporter Gene Chemiluminescent Detection Kit 2.0 (Invitrogen). Finally, the highest SEAP producer was selected and designated as Pp02.

For SEAP production in hydrogels, starter cultures of Pp01 control strain and Pp02 SEAP-producing strain were first grown in YPD at 30° C. Then 4.5×10⁷ overnight cells were embedded in 0.3 g of polymer. The printed and cured cell-laden gels were subsequently incubated in 3 mL of BMGY at 30° C. for cell expansion for 24 hours (cell outgrowth stage defined as round 0, the transfer to and subsequent induction in BMMY for a first production stage as round 1 of reuse, the next transfer to new BMMY as round 2 and so on). It should be noted that the SEAP induction was not initiated at round 0. After 24 hours of incubation, each gel sample was transferred to 3 mL of BMMY (in the presence of the inducer 0.5% methanol) for induction of secreted SEAP expression (round 1). For the liquid culture system, 4.5×10⁶ seeding yeast cells for each strain were transferred to 3 mL of BMGY media and incubated at 30° C. for 24 hours (round 0). Then 4.5×10⁶ cells for each strain were transferred to 3 mL of BMMY medium for protein induction (round 1). Two consecutive reuses after round 1 were performed and samples were proceeded to lyophilization treatment as described in Examples 1 and 2. The preserved samples were next transferred to 3 mL of BMMY media for two additional repetitive uses. All reuse batches were carried out with 30° C. incubation for 48 hours and 0.5% methanol was aseptically added to the culture every 24 hours to maintain protein induction. Each gel sample was washed twice with 800 uL of BMMY media to ensure carry-over of secreted SEAP enzyme and metabolites from the previous batch were not being introduced to the next batch of reuse. Repetitive uses with suspension cultures were performed through subculturing 50 μL of yeast suspension from previous batch to the next batch. Supernatants at 48-hours timepoint were taken from each round of reuse and analyzed by luminescence measurement.

Luminescence was measured with a BioTek Cytation 3 at emission detection range from 500 nm to 600 nm resulting in maximal emission at of 540 nm. SEAP standards were prepared by adding the purified SEAP enzyme (Invitrogen) to the Pp01 control strain spent medium.

α-Amylase Secretion and Production

The plasmids pPICZα and pPICZα-AmyL containing S. cerevisiae α-factor secretion signal sequence were first digested by restriction enzyme Pme I to promote integration into the AOX1 locus. Then the linearized integration cassettes were separately transformed into P. pastoris GS115 by electroporation. YPD agar plates supplemented with 100 μg/mL zeocin were used for selection of zeocin resistant transformants. Zeocin resistance protein binds zeocin antibiotic in a stoichiometric manner. Therefore, selection with a higher concentration of zeocin makes it easy to screen for high-copy number transformed variants. 48 colonies were picked from the agar plate and transferred to a 96-deep-well microplate containing 500 μL of YPD+100 μg/mL zeocin broth medium for each well. Through a series of selections under gradually increased selection pressure (from 100, 500 to 1000 μg/mL zeocin) at 30° C., 10 clones with high growth rate were selected for testing α-amylase production. 500 μL of BMGY and BMMY (with 0.5% methanol) media were used for cell outgrowth and α-amylase expression. After 48 hours of fermentation, supernatants containing secreted α-amylase from the pelleted yeast cells were collected and the enzyme activities were analyzed by measuring the size of the halos forming on starch agar plates (3% agar and 5% soluble starch) and plate-based starch-iodine assay. Finally, the highest α-amylase producer was selected and designated as Pp04.

For evaluation of α-amylase production in hydrogels, started cultures of Pp03 control strain and Pp04 α-amylase-producing strain were first grown in YPD at 30° C. The procedure of preparation of yeast-laden hydrogels and liquid cultures is the same as describe in the above SEAP production section, except that each round of reuse took 24 hours. For plate-based starch-iodine assay, assay reactions were initiated by mixing 40 μL of 5% soluble starch solution and 40 μL of B. licheniformis α-amylase standard (Sigma; product number A4551) solution prepared in Pp03 control strain spent media or 40 μL of supernatants from the pelleted yeast samples. After incubation at 37° C. for 5 minutes, 20 L of HCl was added to the mixture to stop the enzymatic reaction, followed by the addition of 100 μL of iodine-KI reagent (5 mM I₂ and 5 mM KI in DI water). Then 100 μL of the iodine-treated samples were transferred to a transparent flat-bottomed 96-well microplate and the absorbances at 570 nm were recorded using BioTek Cytation 3.

SDS-PAGE Analysis of Secreted Proteins

After fermentation, yeast cells from the cultures were removed by centrifuging at 4000×g for 30 min at 4° C. The supernatants were concentrated using Amicon® Ultra-15 centrifugal filter (Millipore) with a 10 kDa molecular weight cutoff. The concentrated samples were then analyzed by running 10% SDS-PAGE gel and stained with Coomassie Blue.

Results

To test the Bio-POD approach, three proteins of increasing size were selected to demonstrate feasibility and relevance. It has been demonstrated that E. coli-laden hydrogels were capable of secreting the small peptide antibiotic colicin V (around 10 kDa) in a consistent manner (even after lyophilization) over four consecutive re-uses in contrast to that of suspension culture (Examples 1 and 2). Here, the novel use of P. pastoris was evaluated within this gel-based system for secreted placental alkaline phosphatase, α-amylase, and the antibody anti-human epidermal growth factor receptor 2 (Trastuzumab) as model proteins ranging from 60-150 kDa. In each case, a P. pastoris cell line was generated which was capable of modest-level production. P. pastoris-laden hydrogel inks were printed, and protein production pre- and post-lyophilization were tested with repeated use while comparing to a traditional suspension culture.

On-Demand Production of SEAP Using the Bio-POD System

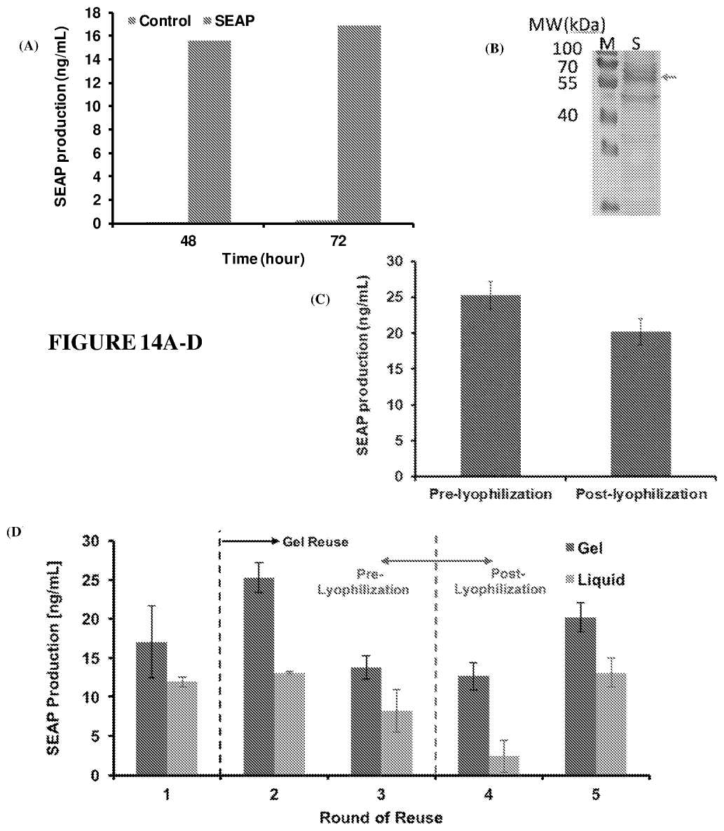

To first evaluate if the F127-BUM hydrogel-based platform was suitable for higher molecular weight protein production using a eukaryotic expression system, P. pastoris was engineered to produce secreted placental alkaline phosphatase (SEAP; a protein around 60 kDa), which is a commonly used reporter enzyme in mammalian cell studies (Cheng 2016). Integration of the SEAP gene expression cassette into P. pastoris followed by screening resulted in isolating strain Pp02 capable of secreting 16 ng/mL of SEAP protein in shake flasks 48 h post-induction (FIG. 14 A and B).

Next, the resulting Pp02 strain was encapsulated in the hydrogel matrix and cells were allowed to proliferate for 24 hours prior to SEAP induction testing (i.e. a round 0 gel use outgrowth step followed by round 1 production stage in which the gels were transferred to induction media). In this experiment, a total of five repetitive use assays (rounds 1-5) were performed, and SEAP production was assessed both pre- and post-preservation by lyophilizing the gel at the midpoint of this reuse experiment. Each round of reuse involved cultivating the gels in BMMY for 48 hours, with media supplemented with an additional 0.5% (v/v) inducer methanol at 24 hours. To showcase the robustness of the gel system, the lyophilization procedure did not include the use of any cryoprotectants, yet included a 10-minutes freezing step in liquid nitrogen prior to vacuum drying. With this simplified procedure, post-lyophilized hydrogel samples (average of round 4 and round 5) retained nearly 90% of pre-lyophilization (average of rounds 1-3) SEAP secretion capacity, with the highest post-lyophilization titer (20.2±1.9 ng/mL) reaching approximately 80% of the maximum pre-lyophilization titer (25.3±1.9 ng/mL) (FIG. 14C). Furthermore, the average titer of round 2-5 of hydrogel reuse (18.0±5.4 ng/mL) was quite similar to the round 1 hydrogel titer (17.1±4.6 ng/mL), thus displaying that the encapsulated cells remained metabolically active despite multiple rounds of reuse and simplified preservation procedures (FIG. 14D).

Finally, these SEAP production values were compared to that of traditional suspension cultures treated in a similar manner. Specifically, the pre- and post-lyophilization average values for suspension cultures were roughly 60% and 45% of the corresponding hydrogel values. Likewise, the maximum pre- and post-lyophilization values for suspension cultures were approximately 52% and 65% of corresponding hydrogel titers, respectively. Collectively, these results demonstrate that the F127-BUM hydrogel matrix enables sustained production and secretion of SEAP by P. pastoris cells with higher titers, more consistency over rounds of reuse, and enhanced cell viability and protein production post-preservation compared to similarly treated liquid suspension cultures.

On-Demand Production of α-Amylase Using the Bio-POD System

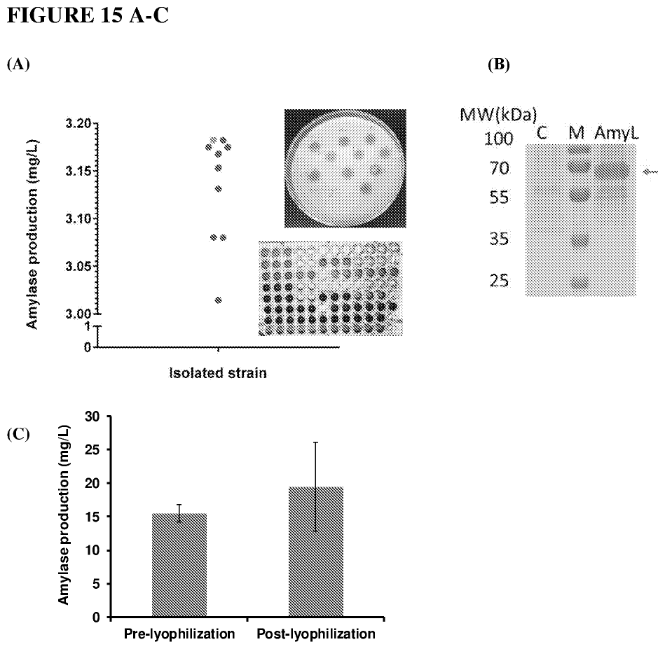

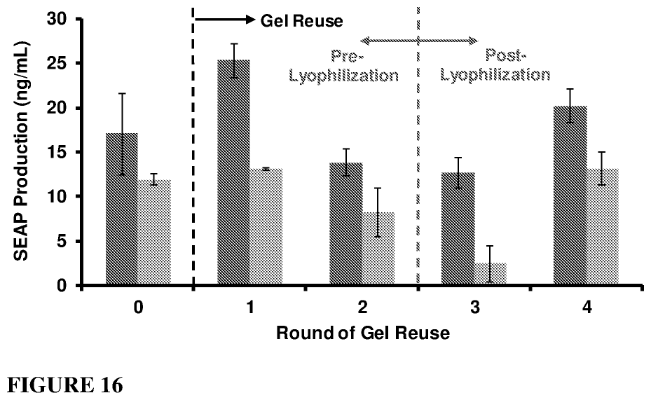

Encouraged by the results with SEAP, the production of another similarly-sized enzyme, namely α-amylase (60 kDa) was next selected, which is a starch hydrolyzing enzyme widely employed in food, detergent and textile industries (Mehta 2016) To first establish a cell line suitable for sustained production and secretion of α-amylase using the Bio-POD technique, P. pastoris was engineered to overproduce and secrete Bacillus licheniformis α-amylase by a similar gene integration and selection strategy (Wang 2015). The resulting strain selected for this work, Pp04, was capable of secreting 3.2 mg/L α-amylase 24 hours post-induction in a 96-deep-well microplate (FIGS. 15A and B).

Pp04 was next encapsulated in the gel matrix, and cells allowed to proliferate for 24 hours prior to induction, and 5 repetitive uses after round 0 were performed in a similar fashion to the SEAP production assays described above (FIG. 14). One deviation from the SEAP experiments was that amylase-secreting cells were grown in BMMY for only 24 hours per round of reuse, as this timeframe was sufficient for amylase production to be detected via a plate-based starch-iodine assay (FIG. 15A). As with the SEAP demonstration, the P. pastoris-laden hydrogels were lyophilized mid-experiment without the addition of cryoprotectants.

Discussion

On-demand protein production enables small batches of biologic products to be produced for precision medicine treatments (Dolsten 2012) as well as in off-the-grid scenarios such as active military missions and in developing nations where existing large-scale manufacturing infrastructure is scarce. While cell immobilization and preservation techniques certainly provide a viable path toward this vision, efforts prior to this report have been limited for P. pastoris. One major hurdle has been the incompatibility of encapsulation materials with the bioprocessing goals of viable cells with high protein titers. In this regard, ionically-crosslinked gels (such as calcium alginate gels), while commonly utilized in many biomedical applications, can hinder diffusion and protein quality/export can depend highly upon the isoelectric point (Wawrzynska 2018). In other cases, the high extrusion temperatures of polymers can damage cells (Ngo 2018), thus further placing a limit on how the cell-laden material can be processed. In contrast to these approaches, the hydrogel approach presented here allows for both ease of processing and production.

The work presented here demonstrates that encapsulation of P. pastoris cells within an F127-BUM hydrogel matrix establishes a platform for on-demand, sustained production of high molecular weight recombinant proteins (demonstrated up to 150 kDa). This system also bypasses the challenges associated with other materials, as the F127-BUM polymer solution undergoes a sol-gel transition around room temperature (17° C.) and affords a shear-thinning gel, thus facilitating the processing to obtain homogenous viable cell distributions. For all three protein products tested, the hydrogel platform displayed a higher absolute titer, a better consistency in round-to-round re-use, and better preservation traits compared to liquid suspension cultures. The sustained production of these distinct protein products from the gels at titers greater than or equal to a liquid culture demonstrates that diffusion of large products through the gel matrix was not a substantial impediment in this platform.

Taken together, these results demonstrate that the Bio-POD technique enables repeated use and preservation for the on-demand production of recombinant proteins using an engineered P. pastoris platform. Based on the reusability and preservation capacity exhibited here, this hydrogel platform can provide for the portable, reusable, and stable production of proteins for on-demand applications. This platform can be coupled with simple, miniaturized downstream protein purification modules (Bareither 2011; Baumann 2017; Millet 2015; Crowell 2018) to yield a field-deployable platform for on-demand production of protein products.

The above specification provides a description of various methods of generating three-dimensional cell cultures or tissues, compositions of the same, methods of use, treatment and diagnosing. Since many embodiments can be made without departing from the spirit and scope of the invention, the invention resides in the claims.

TABLE 1

| Strain | Strain/plasmid Type | Description |
|---|---|---|
| E. coli strain | NEB10β | Δ(ara-leu) 7697 araD139 fhuA |
| | | ΔlacX74 galK16 galE15 e14- |
| | | ϕ80dlacZΔM15 recA1 relA1 endA1 |
| | | nupG rpsL (Str^{R}) rph spoT1 Δ(mrr- |
| | | hsdRMS-mcrBC) |
| | MG1655 | K-12 F− λ− ilvG− rfb-50 rph-1 |
| | BL21(DE3) | E. coli str. B |
| | | F⁻ ompT gal dcm lon hsdS_{B}(r_{B}⁻m_{B}⁻) |
| | | λ(DE3 [lacI lacUV5- |
| | | T7p07 ind1 sam7 nin5]) [malB⁺]_{K-} |
| | | ₁₂(λ^{S}) |
| | CD02 | [MG1655] nfsA::BBa J23119- |
| | | sfGFP; Kan^{R} |
| | MC4100_pHK11 | E. coli MC4100 carries |
| | | pHK11 containing colicin V gene |
| | | cluster cvaAB-cvaC-cvi; Amp^{R} |
| | DH5α_pET28b | E. coli DH5α (ATCC) was |
| | | transformed with pET28b empty |
| | | vector; Kan^{R} |
| | eBL01 | E. coli BL21(DE3) ΔtyrR |
| | eBL04 | [eBL01] pET28-pYIBN-aroG^{(fbr)}- |
| | | B30rbs-tyrA^{(fbr)}-tRRNC; Kan^{R} |
| | eBL0400DT | [eBL04] pCDFDuet-1; Kan^{R}; Spc^{R} |
| | eBL0430D | [eBL04] pCDF-pLPP-B30rbs-hpaB- |
| | | hpaC-T7t; Kan^{R}; Spc^{R} |
| | eBL0432D | [eBL04] pCDF-pLPP-B32rbs-hpaB- |
| | | hpaC-T7t; Kan^{R}; Spc^{R} |
| S. cerevisiae strain | BY4741 | MATα SUC2 gal2 mal2 mel flo1 |
| | | flo8-1 hap1 ho bio1 bio6 his3Δ1 |
| | | leu2Δ0 met15Δ0 ura3Δ0 |
| | CEN.PK2-a | MATa/α; ura3-52/ura3-52; trp1- |
| | | 289/trp1-289; leu2-3_112/leu2- |
| | | 3_112; his3 Δ1/his3 Δ1; MAL2- |
| | | 8C/MAL2-8C; SUC2/SUC2 |
| | S288C | MATα SUC2 gal2 mal2 mel flo1 |
| | | flo8-1 hap1 ho bio1 bio6 |
| | SO992 | MATa ura3 leu2 trp1 his3, can1R, |
| | | ADE+ |
| | yJS001 | [SO992] mfa2::pTEF1-mCherry |
| | JMW001 | [BY4741] trp1::TDH3p-mKate2 |
| | BY4741_BDO | [BY4741] p416-pFBA1-NoxE- |
| | | tIDP1-pTPI1-alsD-tSPG5-pPGK1- |
| | | alsS-tPRM9 |
| | CEN.PK2-a_BDO | [CEN.PK2-a] p416-pFBA1-NoxE- |
| | | tIDP1-pTPI1-alsD-tSPG5-pPGK1- |
| | | alsS-tPRM9 |
| | sBY08 | [BY4741] leu2::pGPD-MjDOD- |
| | | tPRM9 (LEU2 integration with |
| | | URA3 marker) |
| | YSX3 | MATα trp1-112 leu2::LEU2- |
| | | PsXYL1 ura3::URA3-PsXYL2 |
| | | Ty3::NEO-PsXYL3 |
| Plasmids | pET28b-Duet-1 | DNA fragment containing two T7 |
| | | promoters on pRSFDuet-1 was |
| | | cloned into pET28b empty vector |
| | pCDF-Duet-1 | For construction L-DOPA |
| | | producing plasmids |
| | pCDF-pT7-tyrA^{(fbr)}-pT7-aroG^{(fbr)} | FRT flanked kanamycin resistance |
| | | gene was removed from pCDF- |
| | | Kan^{FRT}-tyrA^{(fbr)}-aroG^{(fbr)} |
| | pET28-pT7-aroG^{(fbr)}-tT7 | For construction of pET28-pYIBN- |
| | | aroG^{(fbr)} |
| | pET28-pYIBN-aroG^{(fbr)} | For construction of pET28-pYIBN- |
| | | aroG^{(fbr)}-B30rbs-tyrA^{(fbr)}-tRRNC |
| | pET28-pYIBN-aroG^{(fbr)}-B30rbs- | For tyrosine production |
| | tyrA^{(fbr)}-tRRNC | |
| | pCDF-pLPP-B30rbs-hpaB-hpaC- | For L-DOPA production testing |
| | T7t | |
| | pCDF-pLPP-B32rbs-hpaB-hpaC- | For L-DOPA production testing |
| | T7t | |
| | pCMC0759 | For amplification of MjDOD gene |
| | P416-pGPD-tPRM9 | For construction of p416-pGPD- |
| | | MjDOD-tPRM9 |
| | p416-pGPD-MjDOD-tPRM9 | For generating a URA3 integrative |
| | | cassette employed for betaxanthins |
| | | production |

TABLE 2

| Primer | Primer | |
|---|---|---|
| ID | Description | Sequence (5′->3′) |
| P1 | tyrR_delFwd | TTACGCCGAAGTGCCCGTTTTTCCGTCTTTGTGTCAATGATTGTTGAC |
| | | AGATTCCGGGGATCCGTCGACC (SEQ ID NO: 1) |
| P2 | tyrR_delRvs | CATCAGGCATATTCGCGCTTACTCTTCGTTCTTCTTCTGACTCAGACC |
| | | ATGTGTAGGCTGGAGCTGCTTCG (SEQ ID NO: 2) |
| P3 | tyrR_delFwd1 | CAAAACGCCCAGCGAAAAATAATGCAATATCGGGTGCTGACCGGATAT |
| | | CTTTACGCCGAAGTGCCCG (SEQ ID NO: 3) |
| P4 | tyrR_delRvs1 | AGCTCTGGCTGTACTGAAAGCATAATTTAATATGCCTGATGGTGTTGC |
| | | ACCATCAGGCATATTCGCGCTTACTC (SEQ ID NO: 4) |
| P5 | SeqtyrRf | GATTTCCGTCGTCAGCTTATC (SEQ ID NO: 5) |
| P6 | SeqtyrRr | CAGCTGGTGGATGAAATCAC (SEQ ID NO: 6) |
| P7 | TyrAroCDFf | TGGTGTCCGACAGCCTGCATTAGGAAAT (SEQ ID NO: 7) |
| P8 | TyrAroCDFr | CGCTTATAGTAACGTTTGATTAACG (SEQ ID NO: 8) |
| P9 | CDFTyrAroF | CTGGCGTTAATCAAACGTTACTATAAGCGTTTC (SEQ ID NO: 9) |
| P10 | CDFTyrAroR | ATTTCCTAATGCAGGCTGTCGGACACCATCGAATGGCGCAAAA (SEQ |
| | | ID NO: 10) |
| P11 | ACYCDuetUP1_F | GGATCTCGACGCTCTCCCT (SEQ ID NO: 11) |
| P12 | T7 terminatorR | GCTAGTTATTGCTCAGCGGTG (SEQ ID NO: 12) |
| P13 | p28Duet1F | CTGCTGCCACCGCTGAGCA (SEQ ID NO: 13) |
| P14 | p28Duet1R | AGTCGCATAAGGGAGAGCGTC (SEQ ID NO: 14) |
| P25 | AroGfbrp21F | TGTTTAACTTTAAGAAGGAGATATACATATGAATTATCAGAACGACGA |
| | | TTTACGCATC (SEQ ID NO: 15) |
| P26 | AroGfbrp21R | CGCAAGCTTGTCGACGGAGCTCGAATTCTTACCCGCGACGCGCTTTTA |
| | | (SEQ ID NO: 16) |
| P27 | p28AroGF | GAATTCGAGCTCCGTCGA (SEQ ID NO: 17) |
| P28 | p28AroGR | ATGTATATCTCCTTCTTAAAGTTAAACAAAATTATTTC (SEQ ID |
| | | NO: 18) |
| P33 | yibNf | AAATTGCATTCCAGTTAACGCG (SEQ ID NO: 19) |
| P34 | yibNAroGr | TGCGTAAATCGTCGTTCTGATAATTCATGGGGGGTAACAACTCCC |
| | | (SEQ ID NO: 20) |
| P35 | yibNAroG1f | GTTACCCCCCATGAATTATCAGAACGACGATTTACGCATC (SEQ ID |
| | | NO: 21) |
| P36 | yibNAroG1r | GGAAGAGAGTCAATTCAGGGTGGTGAATTTACCCGCGACGCGCTTTTA |
| | | (SEQ ID NO: 22) |
| P37 | p28yibNAroGf | CGCGGGTAAATTCACCACCCTGAATTGACTC (SEQ ID NO: 23) |
| P38 | p28yibNAroGr | AGCCAGCGCGTTAACTGGAATGCAATTTTGAGCGCAACGCAATTAATG |
| | | TAAG (SEQ ID NO: 24) |
| P39 | p28ATf | ATCCTTAGCGAAAGCTAAGGATTTTTTTTACCATCTTAGTATATTAGT |
| | | TAAGTATAAG (SEQ ID NO: 25) |
| P40 | p28ATB30r | CTAGTATTTCTCCTCTTTAATCTCTAGAGGAAGAGAGTCAATTCAG |
| | | (SEQ ID NO: 26) |
| P41 | yibNtyrAr2 | TTTCCTAATGCAGGAGTCGCATATTACTGGCGATTGTCATTCGC |
| | | (SEQ ID NO: 27) |
| P42 | B30tyrAF | TCTAGAGATTAAAGAGGAGAAATACTAGATGGTTGCTGAATTGACCGC |
| | | (SEQ ID NO: 28) |
| P43 | rrncterm | CCAGTAATATGCGACTCCTGCATTAGGAAAATCCTTAGCGAAAGCTAA |
| | | GGATTTTTTTTA (SEQ ID NO: 29) |
| P67 | lppB30 | CCCATCAAAAAAATATTCTCAACATAAAAAACTTTGTGTAATACTTGT |
| | | AACGCTTCTAGAGATTAAAGAGGAGAAATACTAG (SEQ ID NO: |
| | | 30) |
| P68 | lppB32 | CCCATCAAAAAAATATTCTCAACATAAAAAACTTTGTGTAATACTTGT |
| | | AACGCTTCTAGAGTCACACAGGAAAGTACTAG (SEQ ID NO: 31) |
| P69 | lppHpaBCB30f | TCTAGAGATTAAAGAGGAGAAATACTAGATGAAACCAGAAGATTTCC |
| | | GC (SEQ ID NO: 32) |
| P70 | lppHpaBCB30r | CAGGCGCGCCGAGCTCGAATTCGGATCCTTAAATCGCAGCTTCCATTT |
| | | CC (SEQ ID NO: 33) |
| P71 | lppHpaBCB32f | CTTCTAGAGTCACACAGGAAAGTACTAGATGAAACCAGAAGATTTCCG |
| | | C (SEQ ID NO: 34) |
| P72 | pCDFlppTcXALf | GGATCCGAATTCGAGCTCG (SEQ ID NO: 35) |
| P73 | pCDFlppTcXALr | AGTTTTTTATGTTGAGAATATTTTTTTGATGGGTGAGCGCAACGCAAT |
| | | ATATGTAAG (SEQ ID NO: 36) |
| P74 | SpeIMjDODf | GGACTAGTATGAAGGGAACCTACTACATCAAC (SEQ ID NO: 37) |
| P75 | EcoRIMjDODr | CGGAATTCTTAGGATCCGTCGGTCTTTTG (SEQ ID NO: 38) |
| P78 | NJM675 | TCAAAAAGATCCATGTATAATCTTCATTATTACAGCCCTCTTGACCTC |
| | | TAATCATGAATGTTCTCGGGTGTCGGGGCTGGCTTAACTATG (SEQ |
| | | ID NO: 39) |
| P79 | NJM676 | TATGTAGATTGCGTATATAGTTTCGTCTACCCTATGAACATATTCCAT |
| | | TTTGTAATTTCGTGTCGGTCAGTGAGCGAGGAAGCGGAAGAG (SEQ |
| | | ID NO: 40) |

TABLE 3

| Strain | Strain/Plasmid Type | Description | Source |
|---|---|---|---|
| E. coli strain | NEB10β | Δ(ara-leu) 7697 | New England Biolabs |
| | | araD139 fhuA ΔlacX74 | |
| | | galK16 galE15 e14- | |
| | | ϕ80dlacZΔM15 recA1 | |
| | | relA1 endA1 nupG rpsL | |
| | | (Str^{R}) rph spoT1 Δ(mrr- | |
| | | hsdRMS-mcrBC) | |
| Pichia pastoris strain | GS115 | his4 | Invitrogen |
| | Pp01 | [GS115] his4::pAOX1- | This study |
| | | HIS4 (his4 integration | |
| | | with HIS4 marker); | |
| | | control strain for SEAP | |
| | | production study | |
| | Pp02 | [GS115] his4::pAOX1- | This study |
| | | tSEAP-tAOX1-HIS4 | |
| | | (his4 integration with | |
| | | HIS4 marker); SEAP- | |
| | | producing strain | |
| | Pp03 | [GS115] AOX1:pAOX1- | This study |
| | | tAOX1-ZeoR (AOX1 | |
| | | integration with Zeocin | |
| | | resistance gene); control | |
| | | strain for amylase | |
| | | production study | |
| | Pp04 | [GS115] AOX1:pAOX1- | This study |
| | | AmyL-tAOX1-ZeoR | |
| | | (AOX1 integration with | |
| | | Zeocin resistance gene); | |
| | | Amylase-producing | |
| | | strain | |
| Plasmids | pPIC9 | For construction of | Invitrogen |
| | | plasmid pPIC9-SEAP | |
| | pPICZalphaB-SapL3 | For construction of | Addgene (Plasmid |
| | | plasmid pPICZα_AmyL | #78171) |
| | pPICZα | SapL3 gene, C-myc | This study |
| | | epitope tag and C- | |
| | | terminal polyhistidine | |
| | | (6xHIS) tag were | |
| | | removed from | |
| | | pPICZalphaB-SapL3 | |
| | pPIC9-SEAP | For generating a HIS4 | This study |
| | | integrative cassette | |
| | | employed for SEAP | |
| | | production | |
| | pPICZα_AmyL | For generating an | This study |
| | | integrative cassette | |
| | | containing Zeocin | |
| | | resistance gene | |
| | | employed for amylase | |
| | | production | |

TABLE 4

| ID | Description | Sequence (5′->3′) |
|---|---|---|
| P1 | tSEAPpPIC9_F | CTCTCGAGAAAAGAGAGGCTGAAGCT - |
| | | ATCATCCCAGTTGAGGAGGAGAACC |
| | | (SEQ ID NO: 45) |
| P2 | tSEAPpPIC9_R | GAGGAACAGTCATGTCTAAGGCGAATTA - |
| | | GTCGGTGGTGCCGGC (SEQ ID NO: |
| | | 46) |
| P3 | pPICSSEAP_P | TAATTCGCCTTAGACATGACTG (SEQ ID |
| | | NO: 47) |
| P4 | pPIC9SEAP_R | AGCTTCAGCCTCTCTTTTC (SEQ ID |
| | | NO: 48) |
| P5 | pPICalpha_F | GTTTGTAGCCTTAGACATGACTG (SEQ |
| | | ID NO: 49) |
| P6 | pPICalpha_R | AGCTTCAGCCTCTCTTTTCTC (SEQ ID |
| | | NO: 50) |
| P7 | pZ_Gib | ATCTCTCGAGAAAAGAGAGGCTGAAGCT- |
| | | GTTTGTAGCCTTAGACATGACTGTTCCT |
| | | (SEQ ID NO: 51) |
| P8 | AmyL_F | GGTATCTCTCGAGAAAAGAGAGGCTGAAGC |
| | | T-GCTAATTTGAATGGTACTTTGATG |
| | | (SEQ ID NO: 52) |
| P9 | AmyL_R | GAGGAACAGTCATGTCTAAGGCTACAAAC- |
| | | TTATCTTTGAACATAAATAGAAACAGAAC |
| | | (SEQ ID NO: 53) |

TABLE 5

| Description | Sequence (5′->3′) |
|---|---|
| CO_amyL | GCTAATTTGAATGGTACTTTGATGCAGTATTTCGAGTGGTACATGCCTAACGAC |
| | GGACAGCACTGGAAGAGATTGCAGAACGACTCCGCCTACTTGGCTGAGCACGGA |
| | ATTACTGCTGTCTGGATCCCTCCAGCTTACAAGGGAACTTCTCAGGCTGACGTT |
| | GGTTACGGTGCTTACGACTTGTACGACCTTGGTGAGTTCCACCAAAAAGGTACT |
| | GTCCGTACCAAATATGGTACCAAGGGTGAGTTGCAGTCCGCCATTAAGTCCTTG |
| | CACTCCAGAGACATCAACGTCTACGGTGACGTTGTCATCAACCACAAGGGTGGT |
| | GCCGATGCCACTGAAGATGTTACTGCTGTCGAGGTCGACCCAGCTGATAGAAAC |
| | CGTGTCATCTCCGGAGAGCACAGAATCAAGGCTTGGACCCATTTCCATTTCCCA |
| | GGTCGTGGTTCCACCTACTCCGACTTCAAATGGCACTGGTACCACTTCGATGGT |
| | ACCGACTGGGACGAGTCCAGAAAATTGAACCGTATTTACAAGTTCCAAGGTAAA |
| | GCCTGGGACTGGGAGGTTTCCAATGAGAACGGTAATTATGATTACTTGATGTAC |
| | GCTGACATTGACTACGATCACCCAGATGTCGCTGCTGAGATCAAGAGATGGGGT |
| | ACCTGGTACGCCAACGAGCTTCAGTTGGACGGTTTCCGTTTGGACGCCGTCAAG |
| | CACATCAAATTTTCTTTCTTGAGAGACTGGGTCAACCACGTCAGAGAAAAGACC |
| | GGTAAGGAGATGTTCACCGTCGCCGAGTACTGGCAGAACGATCTTGGTGCTTTG |
| | GAAAACTATTTGAACAAGACTAACTTTAACCATTCTGTTTTCGACGTTCCACTT |
| | CACTACCAGTTTCATGCCGCCTCTACCCAGGGTGGTGGTTACGACATGAGAAAG |
| | TTGTTGAACTCCACCGTTGTCTCCAAGCACCCTCTTAAGGCCGTTACCTTTGTC |
| | GACAATCACGACACCCAGCCTGGTCAATCCTTGGAGTCCACTGTTCAGACTTGG |
| | TTCAAGCCATTGGCTTACGCCTTTATTTTGACTAGAGAGTCCGGATACCCACAG |
| | GTTTTCTACGGTGACATGTACGGTACCAAAGGAGACTCCCAAAGAGAGATTCCT |
| | GCTTTGAAGCATAAGATCGAACCTATTTTGAAGGCTCGTAAACAGTACGCCTAC |
| | GGAGCTCAGCACGACTACTTCGATCACCACGATATCGTCGGTTGGACTAGAGAG |
| | GGAGACTCTTCTGTCGCCAACTCTGGTTTGGCCGCTTTGATTACTGATGGTCCA |
| | GGAGGTGCCAAGAGAATGTACGTCGGACGTCAGAACGCTGGTGAGACCTGGCAC |
| | GACATTACCGGTAACAGATCCGAGCCAGTCGTTATCAACTCCGAGGGATGGGGT |
| | GAGTTCCATGTTAACGGTGGTTCTGTTTCTATTTATGTTCAAAGATAA (SEQ |
| | ID NO: 54) |

### REFERENCES

1. S. Y. Lee et al., Nat. Catal. 2, 18-33 (2019).
2. J. M. Clomburg, A. M. Crumbley, R. Gonzalez, Science 355, aag0804 (2017).
3. L. T. Cordova, H. S. Alper, Curr. Opin. Chem. Biol. 35, 37-42 (2016).
4. S.-F. Yuan, H. S. Alper, Microb. Cell Fact. 18, 46 (2019).
5. N. S. McCarty, R. Ledesma-Amaro, Trends Biotechnol. 37, 181-197 (2019).
6. H. Zhang, X. Wang, Metab. Eng. 37, 114-121 (2016).
7. G. W. Roell et al., Microb. Cell Fact. 18, 35 (2019).
8. P. Bittihn, M. O. Din, L. S. Tsimring, J. Hasty, Curr. Opin. Microbiol. 45, 92-99 (2018).
9. J. A. Jones, X. Wang, Curr. Opin. Biotechnol. 53, 33-38 (2018).
10. K. Zhou, K. Qiao, S. Edgar, G. Stephanopoulos, Nat. Biotechnol. 33, 377 (2015).
11. H. Zhang, B. Pereira, Z. Li, G. Stephanopoulos, Proc. Natl. Acad. Sci. 112, 8266 (2015).
12. R. Niwas et al., J. Basic Microbiol. 54, 1233-1239 (2014).
13. V. Kumaravel, S. R. Gopal, Biotechnol. Appl. Biochem. 57, 97-103 (2010).
14. J. L. Connell, E. T. Ritschdorff, M. Whiteley, J. B. Shear, Proc. Natl. Acad. Sci. 110, 18380 (2013).
15. M. Schaffner, P. A. Rühs, F. Coulter, S. Kilcher, A. R. Studart, Sci. Adv. 3, eaao6804 (2017).
16. A. Saha et al., ACS Appl. Mater. Interfaces 10, 13373-13380 (2018).
17. P. S. J. Cheetham, K. W. Blunt, C. Bocke, Biotechnol. Bioeng. 21, 2155-2168 (1979).
18. M. Köpke et al., Appl. Environ. Microbiol. 77, 5467 (2011).
19. C. Miguelez, A. Benazzouz, L. Ugedo, P. De Deurwaerdere, Front. Cell. Neurosci. 11, (2017).
20. M. Deaner, A. Holzman, H. S. Alper, Biotechnol. J. 13, 1700582 (2018).
21. L. Gilson, H. K. Mahanty, R. Kolter, J. Bacteriol. 169, 2466 (1987).
22. C. M. Agapakis, P. M. Boyle, P. A. Silver, Nat. Chem. Biol. 8, 527 (2012).
23. P. S. Grewal, C. Modavi, Z. N. Russ, N. C. Harris, J. E. Dueber, Metab. Eng. 45, 180-188 (2018).
24. Y.-S. Jin, H. Ni, J. M. Laplaza, T. W. Jeffries, Appl. Environ. Microbiol. 69, 495 (200
25. van Dijken, J. P. et al. An interlaboratory comparison of physiological and genetic properties of four Saccharomyces cerevisiae strains. Enzyme Microb. Tech. 26, 706-714 (2000).
26. Mumberg, D., Müller, R. & Funk, M. Regulatable promoters of Saccharomyces cerevisiae: comparison of transcriptional activity and their use for heterologous expression. Nucleic Acids Res. 22, 5767-5768 (1994).
27. Wu, J., Zhou, T., Du, G., Zhou, J. & Chen, J. Modular Optimization of Heterologous Pathways for De Novo Synthesis of (2S)-Naringenin in Escherichia coli. PLOS ONE 9, e101492 (2014).
28. Ji, X.-J., Huang, H. & Ouyang, P.-K. Microbial 2,3-butanediol production: A state-of-the-art review. Biotechnol. Adv. 29, 351-364 (2011).
29. Gonzalez, E. et al. Role of Saccharomyces cerevisiae Oxidoreductases Bdh1p and Ara1p in the Metabolism of Acetoin and 2,3-Butanediol. Appl. Environ. Microbiol. 76, 670-679 (2010).
30. Broadley, K. J. The vascular effects of trace amines and amphetamines. Pharmacol. Ther 125, 363-375 (2010).
31. Surwase, S. N. & Jadhav, J. P. Bioconversion of 1-tyrosine to 1-DOPA by a novel bacterium Bacillus sp. JPJ. Amino Acids 41, 495-506 (2011).
32. Munoz, A. J. et al. Metabolic engineering of Escherichia coli for improving 1-3,4-dihydroxyphenylalanine (1-DOPA) synthesis from glucose. J. Ind. Microbiol. Biotechnol. 38, 1845 (2011).
33. Yuan, S.-F. et al. Production of optically pure 1(+)-lactic acid from waste plywood chips using an isolated thermotolerant Enterococcus faecalis SI at a pilot scale. J. Ind. Microbiol. Biotechnol. 45, 961-970 (2018).
34. Yuan, S.-F., Guo, G.-L. & Hwang, W.-S. Ethanol production from dilute-acid steam exploded lignocellulosic feedstocks using an isolated multistress-tolerant Pichia kudriavzevii strain. Microb. Biotechnol. 10, 1581-1590 (2017).
35. Yuan, S.-F. & Alper, H. S. Metabolic engineering of microbial cell factories for production of nutraceuticals. Microb. Cell Fact. 18, 46 (2019).
36. Nakagawa, A. et al. A bacterial platform for fermentative production of plant alkaloids. Nat. Commun. 2, 326 (2011).
37. Kim, S. C., Min, B. E., Hwang, H. G., Seo, S. W. & Jung, G. Y. Pathway optimization by re-design of untranslated regions for L-tyrosine production in Escherichia coli. Sci. Rep. 5, 13853-13853 (2015).
38. Huang, Q., Lin, Y. & Yan, Y. Caffeic acid production enhancement by engineering a phenylalanine over-producing Escherichia coli strain. Biotechnol. Bioeng. 110, 3188-3196 (2013).
39. Santos, C. N. S. & Stephanopoulos, G. Melanin-Based High-Throughput Screen for L-Tyrosine Production in Escherichia coli. Appl. Environ. Microbiol. 74, 1190 (2008).
40. Gilson, L., Mahanty, H. K. & Kolter, R. Four plasmid genes are required for colicin V synthesis, export, and immunity. J. Bacteriol. 169, 2466 (1987).
41. Cascales, E. et al. Colicin Biology. Microbiol. Mol. Biol. Rev. 71, 158 (2007).
42. Gérard, F., Pradel, N. & Wu, L.-F. Bactericidal Activity of Colicin V Is Mediated by an Inner Membrane Protein, SdaC, of Escherichia coli. J. Bacteriol. 187, 1945 (2005).
43. Eş, I., Vieira, J. D. G. & Amaral, A. C. Principles, techniques, and applications of biocatalyst immobilization for industrial application. Appl. Microbiol. Biotechnol. 99, 2065-2082 (2015).
44. Alonso, S. in Novel Food Fermentation Technologies. (eds. K. S. Ojha & B. K. Tiwari) 7-33 (Springer International Publishing, Cham; 2016).
45. Martins, N., Roriz, C. L., Morales, P., Barros, L. & Ferreira, I. C. F. R. Coloring attributes of betalains: a key emphasis on stability and future applications. Food Funct. 8, 1357-1372 (2017).
46. DeLoache, W. C. et al. An enzyme-coupled biosensor enables (S)-reticuline production in yeast from glucose. Nat. Chem. Biol. 11, 465 (2015).
47. Gírio, F. M. et al. Hemicelluloses for fuel ethanol: A review. Bioresour Technol. 101, 4775-4800 (2010).
48. Kwak, S. & Jin, Y.-S. Production of fuels and chemicals from xylose by engineered Saccharomyces cerevisiae: a review and perspective. Microb. Cell Fact. 16, 82 (2017).
49. Moysés, D. N., Reis, V. C. B., de Almeida, J. R. M., de Moraes, L. M. P. & Torres, F. A. G. Xylose Fermentation by Saccharomyces cerevisiae: Challenges and Prospects. Int. J. Mol. Sci. 17, 207-207 (2016).
50. Karl Francis, Bernhard O. Palsson. Effective intercellular communication distances are determined by the relative time constants for cyto/chemokine secretion and diffusion. PNAS 94 (23) 12258-12262 (1997).
51. R. Singh, M. Kumar, A. Mittal, P. K. Mehta, 3 Biotech 2016, 6, 174.
52. F. M. Wurm, Nature Biotechnology 2004, 22, 1393-1398.
53. L. Rieder, N. Teuschler, K. Ebner, A. Glieder, Chapter 1.3—Eukaryotic Expression Systems for Industrial Enzymes, Industrial Enzyme Applications, Wiley Online Library 2019, pp. 47-69.
54. R. R. Singhania, A. K. Patel, L. Thomas, M. Goswami, B. S. Giri, A. Pandey, Chapter 13—Industrial Enzymes, in: Pandey, A., Höfer, R., Taherzadeh, M., Nampoothiri, K. M., Larroche, C. (Eds.), Industrial Biorefineries & White Biotechnology, Elsevier, Amsterdam 2015, pp. 473-497.
55. S. S. Dewan, BCC Research 2018.
56. S. S. Dewan, BCC Research 2017.
57. A. M. Vieira Gomes, T. Souza Carmo, L. Silva Carvalho, F. Mendonça Bahia, N. S. Parachin, Microorganisms 2018, 6, 38.
58. R. Baghban, S. Farajnia, M. Rajabibazl, Y. Ghasemi, A. Mafi, R. Hoseinpoor, L. Rahbarnia, M. Aria, Molecular Biotechnology 2019, 61, 365-384.
59. V. Juturu, J. C. Wu, ChemBioChem 2018, 19, 7-21.
60. K. P. Clapp, A. Castan, E. K. Lindskog, Chapter 24—Upstream Processing Equipment, in: Jagschies, G., Lindskog, E., Ła̧cki, K., Galliher, P. (Eds.), Biopharmaceutical Processing, Elsevier 2018, pp. 457-476.
61. N. Srivastava, M. Srivastava, P. K. Mishra, V. K. Gupta, G. Molina, S. Rodriguez-Couto, A. Manikanta, P. W. Ramteke, Renewable and Sustainable Energy Reviews 2018, 82, 2379-2386.
62. J. F Buyel, R. M. Twyman, R. Fischer, Biotechnology Advances 2017, 35, 458-465.
63. J. E. Fischer, A. Glieder, Current Opinion in Biotechnology 2019, 59, 175-181.
64. Z. Kang, H. Huang, Y. Zhang, G. Du, J. Chen, World Journal of Microbiology and Biotechnology 2016, 33, 19.
65. S. Katla, B. Karmakar, S. R. R. Tadi, N. Mohan, B. Anand, U. Pal, S. Sivaprakasam, Journal of Applied Microbiology 2019, 126, 1438-1453.
66. J. Moreno-García, T. Garcia-Martínez, J. C. Mauricio, J. Moreno, Frontiers in Microbiology 2018, 9.
67. D. Martínez, C. Menéndez, F. M. Echemendia, E. R. Pérez, L. E. Trujillo, A. Sobrino, R. Ramirez, Y. Quintero, L. Hernández, Microbial Cell Factories 2014, 13, 87.
68. Y.-M. Li, J.-Q. Gao, X.-Z. Pei, C. Du, C. Fan, W.-J. Yuan, F.-W. Bai, Microbial Cell Factories 2019, 18, 27.
69. F. Ansari, P. Grigoriev, S. Libor, I. E. Tothill, J. J. Ramsden, Biotechnology and Bioengineering 2009, 102, 1505-1512.
70. S.-M. Taghizadeh, A. Ebrahiminezhad, M. B. Ghoshoon, A. Dehshahri, A. Berenjian, Y. Ghasemi, Nanomaterials (Basel) 2020, 10, 111.
71. T. G. Johnston, S.-F. Yuan, J. M. Wagner, X. Yi, A. Saha, P. Smith, A. Nelson, H. S. Alper, Nature Communications 2020, 11, 563.
72. D. G. Gibson, L. Young, R.-Y. Chuang, J. C. Venter, C. A. Hutchison Iii, H. O. Smith, Nature Methods 2009, 6, 343.
73. J. K. Cheng, H. S. Alper, ACS Synthetic Biology 2016, 5, 1455-1465.
74. H. Heimo, K. Palmu, I. Suominen, Protein Expression and Purification 1998, 12, 85-92.
75. D. Mehta, T. Satyanarayana, Frontiers in Microbiology 2016, 7.
76. J.-R. Wang, Y.-Y. Li, D.-N. Liu, J.-S. Liu, P. Li, L.-Z. Chen, S.-D. Xu, Biomed Res Int 2015, 2015, 248680-248680.
77. M. Dolsten, M. Søgaard, Clinical and Translational Medicine 2012, 1, 7.
78. E. Wawrzyńska, D. Kubies, Physiol Res 2018, 67, 5319-5334.
79. T. D. Ngo, A. Kashani, G. Imbalzano, K. T. Q. Nguyen, D. Hui, Composites Part B: Engineering 2018, 143, 172-196.
80. R. Bareither, D. Pollard, Biotechnology Progress 2011, 27, 2-14.
81. P. Baumann, J. Hubbuch, Engineering in Life Sciences 2017, 17, 1142-1158.
82. L. J. Millet, J. D. Lucheon, R. F. Standaert, S. T. Retterer, M. J. Doktycz, Lab on a Chip 2015, 15, 1799-1811.
83. L. E. Crowell, A. E. Lu, K. R. Love, A. Stockdale, S. M. Timmick, D. Wu, Y. Wang, W. Doherty, A. Bonnyman, N. Vecchiarello, C. Goodwine, L. Bradbury, J. R. Brady, J. J. Clark, N. A. Colant, A. Cvetkovic, N. C. Dalvie, D. Liu, Y. Liu, C. A. Mascarenhas, C. B. Matthews, N. J. Mozdzierz, K. A. Shah, S.-L. Wu, W. S. Hancock, R. D. Braatz, S. M. Cramer, J. C. Love, Nature Biotechnology 2018, 36, 988-995.

1. A composition comprising a 3D printed hydrogel, wherein at least two different populations of cells are embedded in the 3D printed hydrogel.

2. The composition of claim 1, wherein the cells comprise eukaryotes, prokaryotes, or a combination of both.

3. The composition of claim 2, wherein the cells comprise yeast, bacteria, or a combination of both.

4. The composition of claim 1, wherein at least one of the population of cells is capable of producing at least one product.

5. The composition of claim 4, wherein said product comprises a small molecule or a protein.

6. The composition of claim 5, wherein at least both a first and second populations of cells are capable of producing a product.

7. The composition of claim 5, wherein at least one product is capable of being produced at a rate of 50% or higher after lyophilization and rehydration of the composition as compared to production of at least one product prior to lyophilization and rehydration of the composition.

8. The composition of claim 7, wherein at least one product is capable of being produced at a rate of 50% or higher after lyophilization and rehydration of the composition at least a year or more after lyophilization and rehydration.

9. The composition of claim 7, wherein at least one product is capable of being produced at a rate of 50% or higher after more than one lyophilization and rehydration cycle.

10. The composition of claim 6, wherein at least the first and second populations of cells is capable of producing different products.

11. The composition of claim 10, wherein the first population of cells is capable of producing a product which is consumed by the second population of cells.

12. The composition of claim 4, wherein at least one population of cells embedded in the hydrogel is capable of producing a product for at least 7 consecutive days.

13. The composition of claim 1, wherein the cells are spatially organized in the 3D printed hydrogel.

14. The composition of claim 13 wherein the at least two different populations occupy at least two different spatial areas in the hydrogel.

15. The composition of claim 1, wherein the 3D hydrogel comprises a polymer of Formula (I): 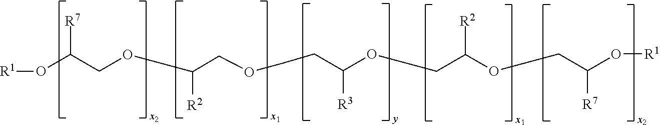
wherein
R¹ is hydrogen or a group having the formula: 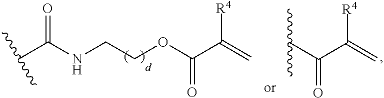
wherein d is 1, 2, 3, 4, 5, 6, 7, 8, 9, 10, 11, or 12; and R⁴ is hydrogen or methyl;
R² is hydrogen or —CH₂OR⁵; wherein R⁵ is C₁₋₁₂alkyl, C₁₋₁₂alkyl-OR⁶ or C₁₋₁₂alkyl-NR⁶₂, wherein each R⁶ is independently hydrogen or C₁₋₁₂alkyl;
R³ is hydrogen or methyl; and
R⁴ is hydrogen or methyl;
wherein R⁷ is —CH₂—O—C₁₋₁₂alkenyl;
y is selected to provide a block polymer with an Mₙ of about 500 to about 50,000;
x¹ and x² are selected to provide a block polymer with an Mₙ of about 100 to about 30,000;
x¹ is from 10-100; and y is from 25-250, provided that y is greater than x¹.

16. The composition of claim 1, wherein the 3D hydrogel comprises a polymer having the structure of Formula (II):

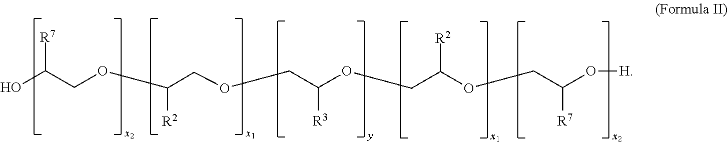

17. The composition of claim 16, wherein:
R² is —CH₂—O—C₁₋₆alkyl; and
R⁷ is —CH₂—O—C₂₋₆alkenyl.

18. The composition of claim 16, wherein the polymer has the structure of Formula (IIa):

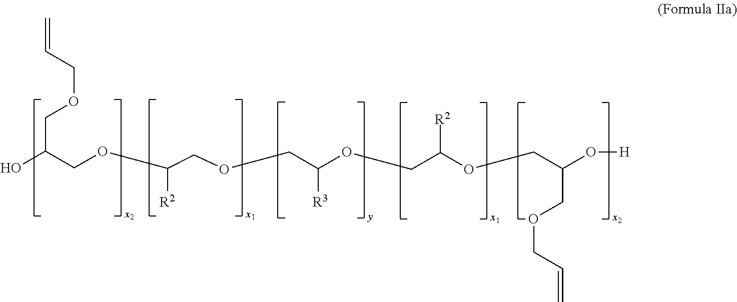

19. The composition of claim 18, wherein R² is —CH₂—O—C₁₋₆alkyl.

20. The composition of claim 16, wherein the polymer has the structure of Formula (IIb):

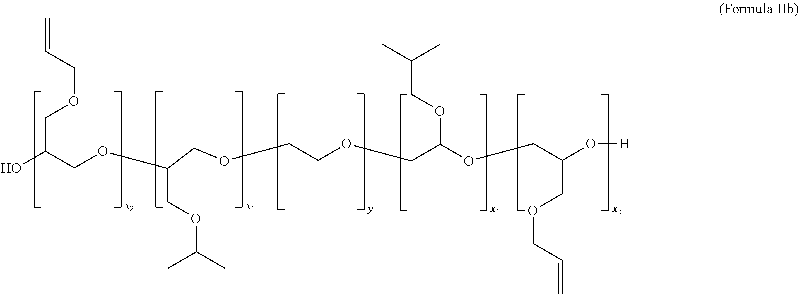

21. The composition of claim 16, wherein the polymer has the structure of Formula (IIc):

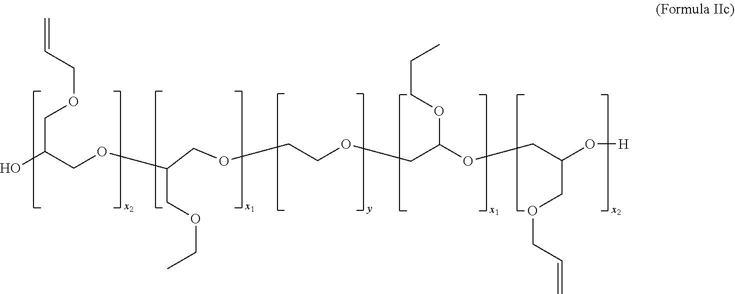

22. The composition of claim 15, wherein the polymer has the structure of Formula (III):

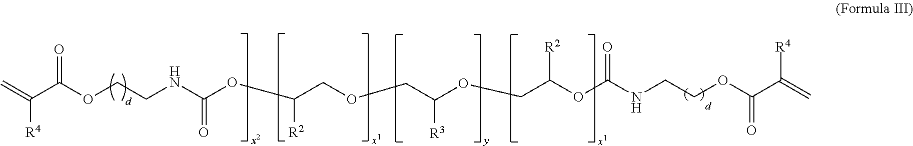

23. The composition of claim 22, wherein R² is —CH₂—O—C₁₋₆alkyl.

24. The composition of claim 22, having the structure of Formula (IIIa):

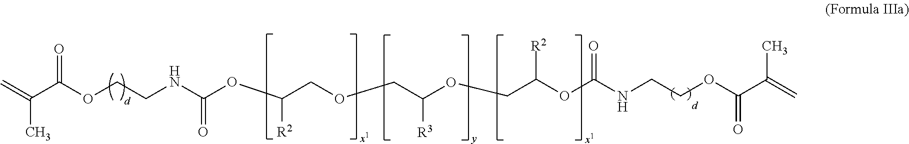

25. The composition of claim 22, wherein the polymer has the structure of Formula (IIIb):

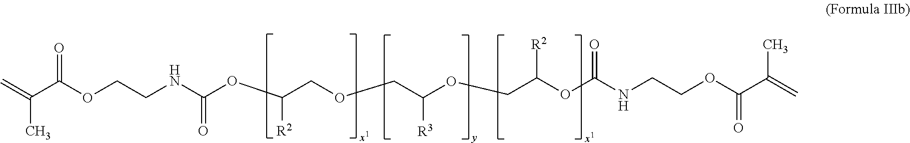

26. The composition of claim 22, wherein the polymer has the structure of Formula (IIIc):

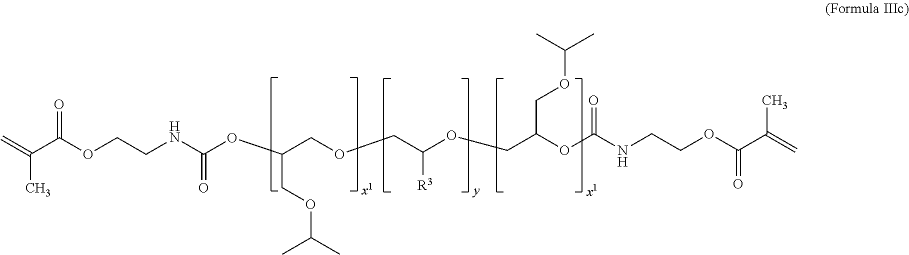

27-81. (canceled)


SEQUENCE LISTING (54 sequences)

SEQ ID NO: 1 - DNA, 70 bp, Artificial Sequence
  Synthetic construct
ttacgccgaagtgcccgtttttccgtctttgtgtcaatgattgttgacagattccggggatccgtcgacc

SEQ ID NO: 2 - DNA, 71 bp, Artificial Sequence
  Synthetic construct
catcaggcatattcgcgcttactcttcgttcttcttctgactcagaccatgtgtaggctggagctgcttcg

SEQ ID NO: 3 - DNA, 67 bp, Artificial Sequence
  Synthetic construct
caaaacgcccagcgaaaaataatgcaatatcgggtgctgaccggatatctttacgccgaagtgcccg

SEQ ID NO: 4 - DNA, 74 bp, Artificial Sequence
  Synthetic construct
agctctggctgtactgaaagcataatttaatatgcctgatggtgttgcaccatcaggcatattcgcgcttactc

SEQ ID NO: 5 - DNA, 21 bp, Artificial Sequence
  Synthetic construct
gatttccgtcgtcagcttatc

SEQ ID NO: 6 - DNA, 20 bp, Artificial Sequence
  Synthetic construct
cagctggtggatgaaatcac

SEQ ID NO: 7 - DNA, 28 bp, Artificial Sequence
  Synthetic construct
tggtgtccgacagcctgcattaggaaat

SEQ ID NO: 8 - DNA, 25 bp, Artificial Sequence
  Synthetic construct
cgcttatagtaacgtttgattaacg

SEQ ID NO: 9 - DNA, 33 bp, Artificial Sequence
  Synthetic construct
ctggcgttaatcaaacgttactataagcgtttc

SEQ ID NO: 10 - DNA, 43 bp, Artificial Sequence
  Synthetic construct
atttcctaatgcaggctgtcggacaccatcgaatggcgcaaaa

SEQ ID NO: 11 - DNA, 19 bp, Artificial Sequence
  Synthetic construct
ggatctcgacgctctccct

SEQ ID NO: 12 - DNA, 21 bp, Artificial Sequence
  Synthetic construct
gctagttattgctcagcggtg

SEQ ID NO: 13 - DNA, 19 bp, Artificial Sequence
  Synthetic construct
ctgctgccaccgctgagca

SEQ ID NO: 14 - DNA, 21 bp, Artificial Sequence
  Synthetic construct
agtcgcataagggagagcgtc

SEQ ID NO: 15 - DNA, 58 bp, Artificial Sequence
  Synthetic construct
tgtttaactttaagaaggagatatacatatgaattatcagaacgacgatttacgcatc

SEQ ID NO: 16 - DNA, 48 bp, Artificial Sequence
  Synthetic construct
cgcaagcttgtcgacggagctcgaattcttacccgcgacgcgctttta

SEQ ID NO: 17 - DNA, 18 bp, Artificial Sequence
  Synthetic construct
gaattcgagctccgtcga

SEQ ID NO: 18 - DNA, 38 bp, Artificial Sequence
  Synthetic construct
atgtatatctccttcttaaagttaaacaaaattatttc

SEQ ID NO: 19 - DNA, 22 bp, Artificial Sequence
  Synthetic construct
aaattgcattccagttaacgcg

SEQ ID NO: 20 - DNA, 45 bp, Artificial Sequence
  Synthetic construct
tgcgtaaatcgtcgttctgataattcatggggggtaacaactccc

SEQ ID NO: 21 - DNA, 40 bp, Artificial Sequence
  Synthetic construct
gttaccccccatgaattatcagaacgacgatttacgcatc

SEQ ID NO: 22 - DNA, 48 bp, Artificial Sequence
  Synthetic construct
ggaagagagtcaattcagggtggtgaatttacccgcgacgcgctttta

SEQ ID NO: 23 - DNA, 31 bp, Artificial Sequence
  Synthetic construct
cgcgggtaaattcaccaccctgaattgactc

SEQ ID NO: 24 - DNA, 52 bp, Artificial Sequence
  Synthetic construct
agccagcgcgttaactggaatgcaattttgagcgcaacgcaattaatgtaag

SEQ ID NO: 25 - DNA, 58 bp, Artificial Sequence
  Synthetic construct
atccttagcgaaagctaaggattttttttaccatcttagtatattagttaagtataag

SEQ ID NO: 26 - DNA, 46 bp, Artificial Sequence
  Synthetic construct
ctagtatttctcctctttaatctctagaggaagagagtcaattcag

SEQ ID NO: 27 - DNA, 44 bp, Artificial Sequence
  Synthetic construct
tttcctaatgcaggagtcgcatattactggcgattgtcattcgc

SEQ ID NO: 28 - DNA, 48 bp, Artificial Sequence
  Synthetic construct
tctagagattaaagaggagaaatactagatggttgctgaattgaccgc

SEQ ID NO: 29 - DNA, 60 bp, Artificial Sequence
  Synthetic construct
ccagtaatatgcgactcctgcattaggaaaatccttagcgaaagctaaggatttttttta

SEQ ID NO: 30 - DNA, 82 bp, Artificial Sequence
  Synthetic construct
cccatcaaaaaaatattctcaacataaaaaactttgtgtaatacttgtaacgcttctagagattaaagaggagaaatactag

SEQ ID NO: 31 - DNA, 80 bp, Artificial Sequence
  Synthetic construct
cccatcaaaaaaatattctcaacataaaaaactttgtgtaatacttgtaacgcttctagagtcacacaggaaagtactag

SEQ ID NO: 32 - DNA, 49 bp, Artificial Sequence
  Synthetic construct
tctagagattaaagaggagaaatactagatgaaaccagaagatttccgc

SEQ ID NO: 33 - DNA, 50 bp, Artificial Sequence
  Synthetic construct
caggcgcgccgagctcgaattcggatccttaaatcgcagcttccatttcc

SEQ ID NO: 34 - DNA, 49 bp, Artificial Sequence
  Synthetic construct
cttctagagtcacacaggaaagtactagatgaaaccagaagatttccgc

SEQ ID NO: 35 - DNA, 19 bp, Artificial Sequence
  Synthetic construct
ggatccgaattcgagctcg

SEQ ID NO: 36 - DNA, 57 bp, Artificial Sequence
  Synthetic construct
agttttttatgttgagaatatttttttgatgggtgagcgcaacgcaattaatgtaag

SEQ ID NO: 37 - DNA, 32 bp, Artificial Sequence
  Synthetic construct
ggactagtatgaagggaacctactacatcaac

SEQ ID NO: 38 - DNA, 29 bp, Artificial Sequence
  Synthetic construct
cggaattcttaggatccgtcggtcttttg

SEQ ID NO: 39 - DNA, 90 bp, Artificial Sequence
  Synthetic construct
tcaaaaagatccatgtataatcttcattattacagccctcttgacctctaatcatgaatgttctcgggtgtcggggctggcttaactatg

SEQ ID NO: 40 - DNA, 90 bp, Artificial Sequence
  Synthetic construct
tatgtagattgcgtatatagtttcgtctaccctatgaacatattccattttgtaatttcgtgtcggtcagtgagcgaggaagcggaagag

SEQ ID NO: 41 - DNA, 24 bp, Artificial Sequence
  Synthetic construct
gagtaattcatacaaaaggactcg

SEQ ID NO: 42 - DNA, 23 bp, Artificial Sequence
  Synthetic construct
tttggcttttaggggtagttttc

SEQ ID NO: 43 - DNA, 46 bp, Artificial Sequence
  Synthetic construct
gtcgtgaaaactacccctaaaagccaaagctagcatgctgctgctg

SEQ ID NO: 44 - DNA, 46 bp, Artificial Sequence
  Synthetic construct
gggcgagtccttttgtatgaattactcgcggccgcttttttccttc

SEQ ID NO: 45 - DNA, 51 bp, Artificial Sequence
  Synthetic construct
ctctcgagaaaagagaggctgaagctatcatcccagttgaggaggagaacc

SEQ ID NO: 46 - DNA, 43 bp, Artificial Sequence
  Synthetic construct
gaggaacagtcatgtctaaggcgaattagtcggtggtgccggc

SEQ ID NO: 47 - DNA, 22 bp, Artificial Sequence
  Synthetic construct
taattcgccttagacatgactg

SEQ ID NO: 48 - DNA, 19 bp, Artificial Sequence
  Synthetic construct
agcttcagcctctcttttc

SEQ ID NO: 49 - DNA, 23 bp, Artificial Sequence
  Synthetic construct
gtttgtagccttagacatgactg

SEQ ID NO: 50 - DNA, 21 bp, Artificial Sequence
  Synthetic construct
agcttcagcctctcttttctc

SEQ ID NO: 51 - DNA, 56 bp, Artificial Sequence
  Synthetic construct
atctctcgagaaaagagaggctgaagctgtttgtagccttagacatgactgttcct

SEQ ID NO: 52 - DNA, 55 bp, Artificial Sequence
  Synthetic construct
ggtatctctcgagaaaagagaggctgaagctgctaatttgaatggtactttgatg

SEQ ID NO: 53 - DNA, 58 bp, Artificial Sequence
  Synthetic construct
gaggaacagtcatgtctaaggctacaaacttatctttgaacataaatagaaacagaac

SEQ ID NO: 54 - DNA, 1452 bp, Artificial Sequence
  Synthetic construct
gctaatttgaatggtactttgatgcagtatttcgagtggtacatgcctaacgacggacagcactggaagagattgcagaacgactccgcctacttggctgagcacggaattactgctgtctggatccctccagcttacaagggaacttctcaggctgacgttggttacggtgcttacgacttgtacgaccttggtgagttccaccaaaaaggtactgtccgtaccaaatatggtaccaagggtgagttgcagtccgccattaagtccttgcactccagagacatcaacgtctacggtgacgttgtcatcaaccacaagggtggtgccgatgccactgaagatgttactgctgtcgaggtcgacccagctgatagaaaccgtgtcatctccggagagcacagaatcaaggcttggacccatttccatttcccaggtcgtggttccacctactccgacttcaaatggcactggtaccacttcgatggtaccgactgggacgagtccagaaaattgaaccgtatttacaagttccaaggtaaagcctgggactgggaggtttccaatgagaacggtaattatgattacttgatgtacgctgacattgactacgatcacccagatgtcgctgctgagatcaagagatggggtacctggtacgccaacgagcttcagttggacggtttccgtttggacgccgtcaagcacatcaaattttctttcttgagagactgggtcaaccacgtcagagaaaagaccggtaaggagatgttcaccgtcgccgagtactggcagaacgatcttggtgctttggaaaactatttgaacaagactaactttaaccattctgttttcgacgttccacttcactaccagtttcatgccgcctctacccagggtggtggttacgacatgagaaagttgttgaactccaccgttgtctccaagcaccctcttaaggccgttacctttgtcgacaatcacgacacccagcctggtcaatccttggagtccactgttcagacttggttcaagccattggcttacgcctttattttgactagagagtccggatacccacaggttttctacggtgacatgtacggtaccaaaggagactcccaaagagagattcctgctttgaagcataagatcgaacctattttgaaggctcgtaaacagtacgcctacggagctcagcacgactacttcgatcaccacgatatcgtcggttggactagagagggagactcttctgtcgccaactctggtttggccgctttgattactgatggtccaggaggtgccaagagaatgtacgtcggacgtcagaacgctggtgagacctggcacgacattaccggtaacagatccgagccagtcgttatcaactccgagggatggggtgagttccatgttaacggtggttctgtttctatttatgttcaaagataa